(19) United States
(12) Patent Application Publication
(10) Pub. No.: US 2025/0241195 A1
(43) Pub. Date: Jul. 24, 2025

(54) COMPOUNDS FOR ELECTRONIC DEVICES

(71) Applicant: Merck Patent GmbH, Darmstadt (DE)

(72) Inventors: Elvira MONTENEGRO, Darmstadt (DE); Jens ENGELHART, Darmstadt (DE); You-Hyun KIM, Darmstadt (DE)

(21) Appl. No.: 18/704,495

(22) PCT Filed: Oct. 26, 2022
(86) PCT No.: PCT/EP2022/079851

§ 371 Date: Apr. 25, 2024

(30) Foreign Application Priority Data
Oct. 29, 2021 (EP) 21205643.6

(51) Int. Cl. H10K 85/60; C07C 211/61; C07D 209/86; C07D 219/02; C07D 265/38; C07D 307/91; C07D 333/76; H10K 50/15

(52) U.S. Cl. CPC H10K 85/633; C07C 211/61; C07D 209/86; C07D 219/02; C07D 265/38; C07D 307/91; C07D 333/76; C07B 2200/05; C07C 2601/04; C07C 2603/18; C07C 2603/74; H10K 50/15; H10K 85/626

(57) ABSTRACT

The present invention relates to a compound of formula (I), to its use in electronic devices, to methods for producing said compound, and to electronic devices containing the compound.

The present application relates to fluorenylamines in which the fluorenyl group has at least two substituents on the benzene rings of the fluorene. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The structure and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. There is an ongoing search for novel compounds for use in these layers, especially hole-transporting compounds and compounds that can serve as hole-transporting matrix material, especially for phosphorescent emitters, in an emitting layer. For this purpose, there is a search in particular for compounds that have a high glass transition temperature, high stability, and high conductivity for holes. A high stability of the compound is a prerequisite for achieving a long lifetime of the electronic device. There is also a search for compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices. However, there remains room for improvement in respect of the abovementioned properties.

It has now been found that fluoreneamines of the formula below which are characterized in that they have at least two substituents on the benzene rings of the fluorene are of excellent suitability for use in electronic devices.

They are especially suitable for use in OLEDs, and even more particularly therein for use as hole transport materials and for use as hole-transporting matrix materials, especially for phosphorescent emitters. The compounds found lead to high lifetime, high efficiency and low operating voltage, in particular high efficiency, of the devices. Further preferably, the compounds found have a high glass transition temperature, high stability, low sublimation temperature, good solubility, good synthetic accessibility and high conductivity for holes.

Without being tied to this theory, it is noted that, according to current state of knowledge, the preferred properties of the compounds are partly caused by their asymmetric structure, meaning that the three groups bonded to the nitrogen atom are not all the same. Especially advantageous properties can be achieved when the compounds have heteroaromatic systems or aromatic systems other than fluorenyl on the amine.

The present application provides a compound of a formula (I)

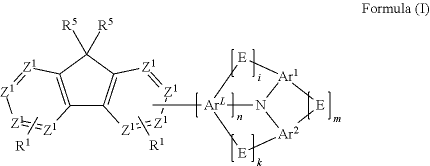

where the variables that occur are defined as follows:
Z¹ is C when an R¹ group or a group

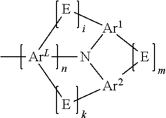

is bonded thereto, and is otherwise the same or different at each instance and is selected from CR² and N;
Ar^{L} is selected from aromatic ring systems which have 6 to 40 aromatic ring atoms and are substituted by R³ radicals and heteroaromatic ring systems which have 5 to 40 aromatic ring atoms and are substituted by R³ radicals;
Ar¹ is selected from aromatic ring systems which have 6 to 40 aromatic ring atoms and are substituted by R⁴ radicals and heteroaromatic ring systems which have 5 to 40 aromatic ring atoms and are substituted by R⁴ radicals;
Ar² is selected from aromatic ring systems which have 6 to 40 aromatic ring atoms and are substituted by R⁴ radicals and heteroaromatic ring systems which have 5 to 40 aromatic ring atoms and are substituted by R⁴ radicals;
E is a single bond or a divalent group selected from —C(R⁶)₂—, —C(R⁶)₂—C(R⁶)₂—, —C(R⁶)═C(R⁶)—, —N(R⁶)—, —O—, and —S—;
R¹ is the same or different at each instance and is selected from F, CN, N(R⁷)₂, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where said alkyl, alkoxy, alkenyl and alkynyl groups and said aromatic ring systems and heteroaromatic ring systems are each substituted by R⁷ radicals;
R⁵ is the same or different at each instance and is selected from straight-chain alkyl or alkoxy groups having 1 to 20 carbon atoms, branched or cyclic alkyl or alkoxy groups having 3 to 20 carbon atoms, and alkenyl or alkynyl groups having 2 to 20 carbon atoms; where said alkyl, alkoxy, alkenyl and alkynyl groups are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR′, P(═O)(R⁷), —O—, —S—, SO or SO₂;
R² is the same or different at each instance and is selected from H, D, F, Cl, Br, I, C(═O)R⁷, CN, Si(R⁷)₃, N(R⁷)₂, NAr¹Ar², P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R³ is the same or different at each instance and is selected from H, D, F, Cl, Br, I, C(═O)R⁷, CN, Si(R⁷)₃, N(R⁷)₂, NAr¹Ar², P(═O)(R⁷)₂, OR′, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁴ is the same or different at each instance and is selected from H, D, F, Cl, Br, I, C(═O)R⁷, CN, Si(R⁷)₃, N(R⁷)₂, P(═O)(R⁷)₂, OR′, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR′, P(═O)(R⁷), —O—, —S—, SO or SO₂;
R⁶ is the same or different at each instance and is selected from H, D, F, Cl, Br, I, C(═O)R⁷, CN, Si(R⁷)₃, N(R⁷)₂, P(═O)(R⁷)₂, OR′, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, C(═O)R⁸, CN, Si(R⁸)₃, N(R⁸)₂, P(═O)(R⁸)₂, OR⁸, S(═O)R⁸, S(═O)₂R⁸, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁷ radicals may be joined to one another and may form a ring; where said alkyl, alkoxy, alkenyl and alkynyl groups and said aromatic ring systems and heteroaromatic ring systems are each substituted by R⁸ radicals; and where one or more CH₂ groups in said alkyl, alkoxy, alkenyl and alkynyl groups may be replaced by —R⁸C═CR⁸—, —C≡C—, Si(R⁸)₂, C═O, C═NR⁸, —C(═O)O—, —C(═O)NR⁸—, NR⁸, P(═O)(R⁸), —O—, —S—, SO or SO₂;
R⁸ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁸ radicals may be joined to one another and may form a ring; and where said alkyl, alkoxy, alkenyl and alkynyl groups, aromatic ring systems and heteroaromatic ring systems may be substituted by one or more radicals selected from F and CN;
m is 0 or 1, where, when m=0, E is absent, and the Ar¹ and Ar² groups are not bonded to one another;
i is 0 or 1, where, in the case that i=0, the E group in question is absent and the Ar^{L} and Ar¹ groups are not bonded to one another;
k is 0 or 1, where, in the case that k=0, the E group in question is absent and the Ar^{L} and Ar² groups are not bonded to one another;
n is 0 or 1, where, when n=0, Ar^{L} is absent, and i and k are both 0, and the fluorene and the amino group in formula (I) are bonded directly to one another;
where the group

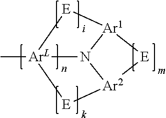

is bonded in the 1, 3 or 4 position of the fluorenyl group of the formula (I),
where the group

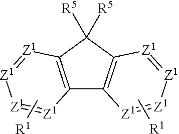

of the formula (I) in the compound is not identical to one or both of the Ar¹ and Ar² groups in the compound; and
where at least one group selected from the Ar¹ and Ar² groups is selected from the following radicals: biphenyl; terphenyl; quaterphenyl; phenanthrenyl; dibenzofuranyl; dibenzothiophenyl; carbazolyl; and phenyl substituted by at least one group selected from fluorenyl, naphthyl, phenanthrenyl, dibenzofuranyl, dibenzothiophenyl, and carbazolyl, where these radicals are each substituted by R⁴ radicals.

In the wording that the group

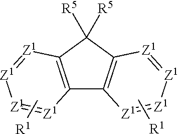

of the formula (I) in the compound is not identical to one or both of the Ar¹ and Ar² groups in the compound, the term “groups” is in each case understood to mean the complete groups including their substituents. What is meant by “not identical” is not just that the groups have different empirical formula, where the term “empirical formula” in this case also includes H and D as different atoms, but also that they are different isomers, as is the case, for example, for o-biphenyl and p-biphenyl.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

The compound of the formula (I) is preferably a monoamine, meaning that it preferably has a single amino group.

In an alternative preferred embodiment, the compound of the formula (I) is a diamine, meaning that it has two and not more than two amino groups. In this case, it is preferable that one R² group in the formula (I) is —NAr¹Ar² or is N(R⁷)₂, more preferably —NAr¹Ar².

In formula (I), Z¹ is preferably C when an R¹ group or the group

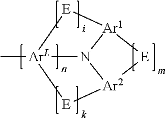

is bonded thereto, and is otherwise preferably CR².

It is further preferable that, in formula (I), not more than three Z¹ groups are N, particularly preferable that not more than two Z¹ groups are N, very particularly preferable that not more than one Z¹ group is N, and most preferable that there is no Z¹ group which is N.

In a preferred embodiment, the group

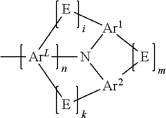

is bonded in the 4 position of the fluorenyl group of the formula (I).

In an alternative preferred embodiment, the abovementioned group is bonded in the 3 position of the fluorenyl group of the formula (I).

In an alternative preferred embodiment, the abovementioned group is bonded in the 1 position of the fluorenyl group of the formula (I).

It is particularly preferable that the abovementioned group is bonded in the 4 position of the fluorenyl group of the formula (I).

Ar^{L} is preferably the same or different at each instance and is selected from phenyl, biphenyl, naphthyl and fluorenyl, each substituted by R³ radicals; and is even more preferably selected from phenyl and biphenyl, most preferably phenyl, substituted by R³ radicals, where R³ in this case is preferably the same or different at each instance and is selected from H and D, and is more preferably H.

Ar^{L} is preferably selected from the following groups:

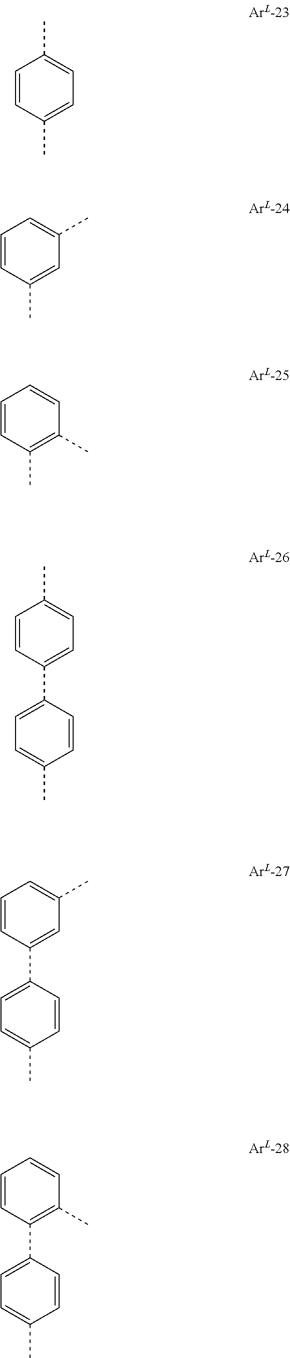

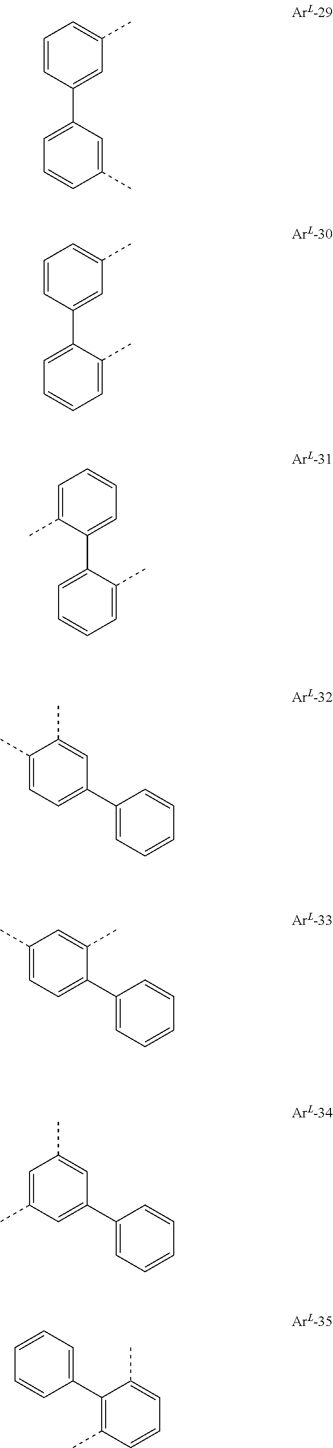

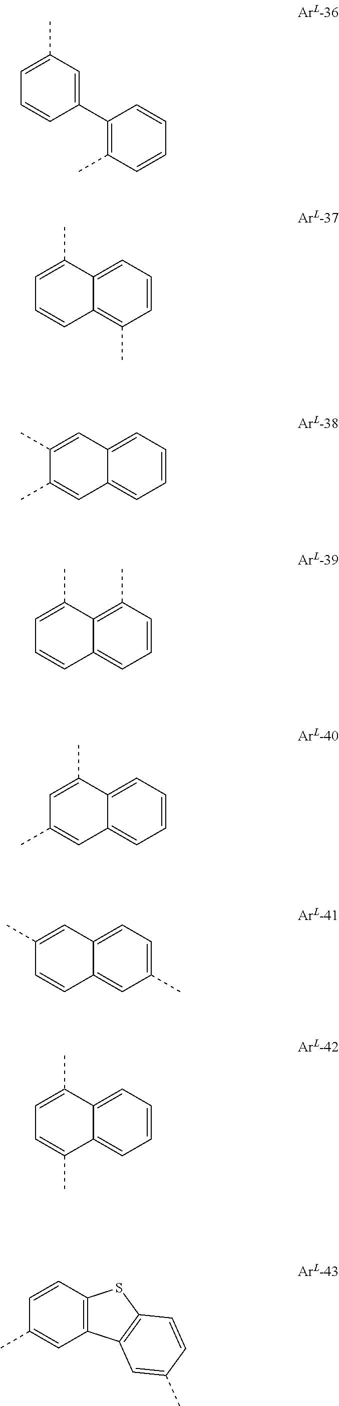

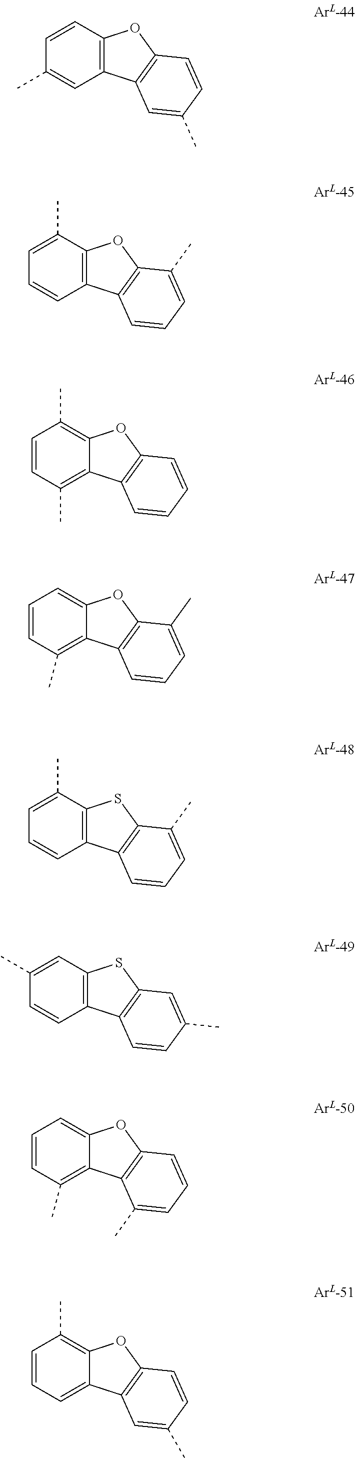

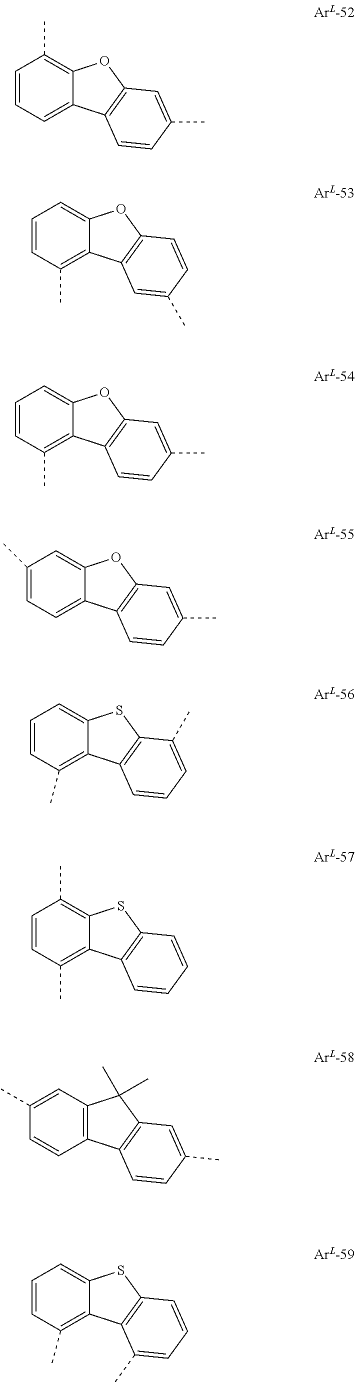

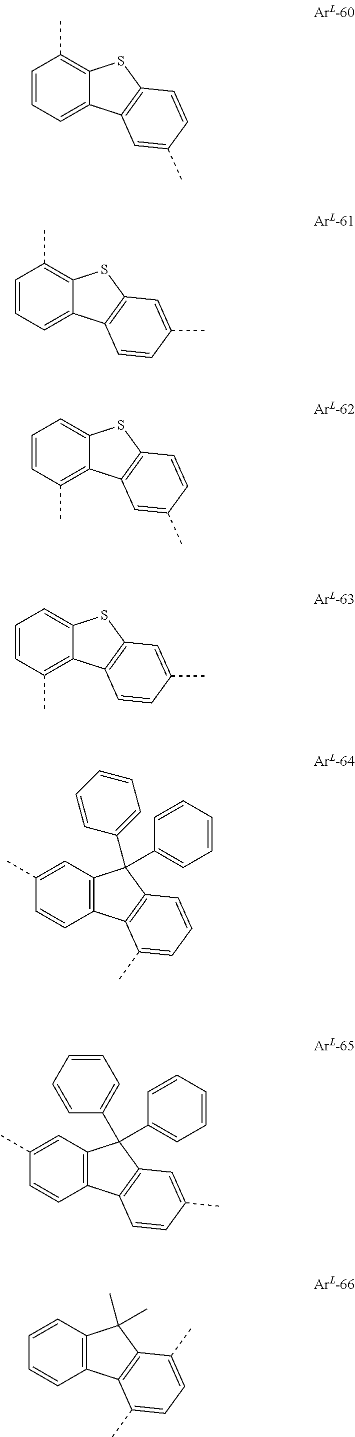

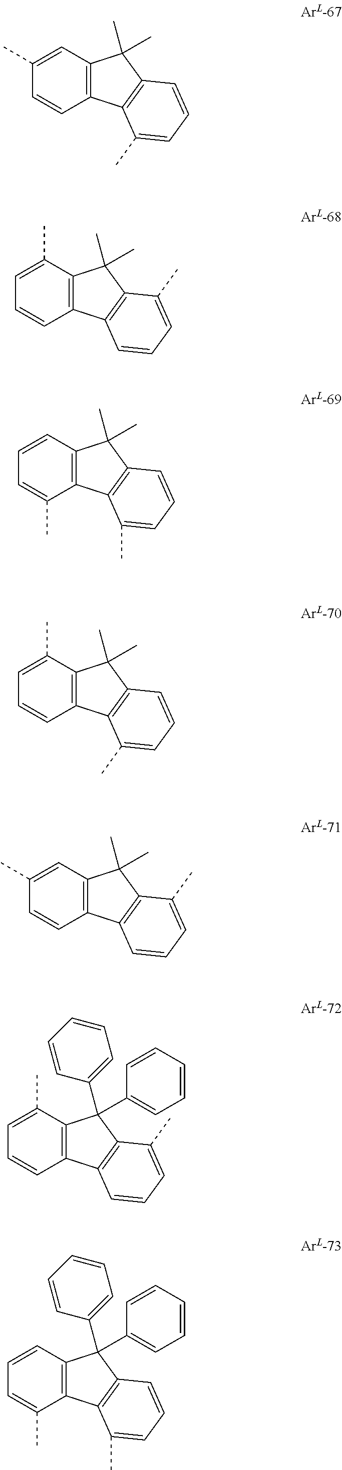

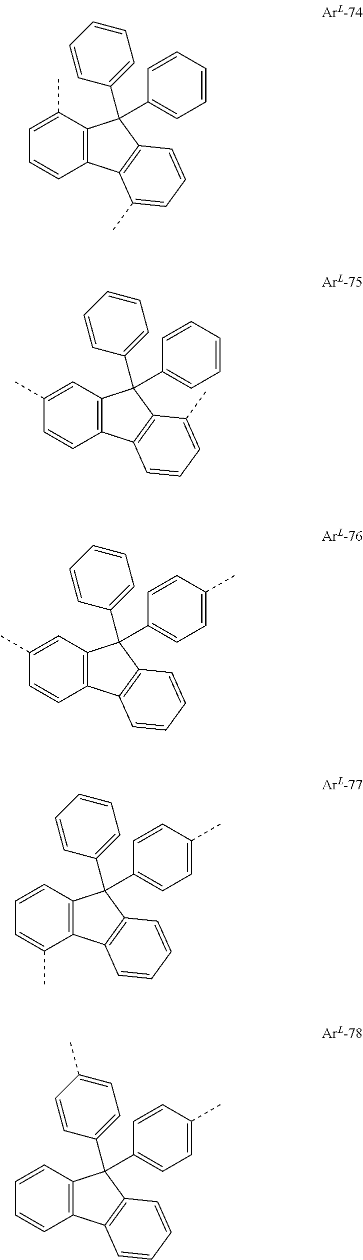

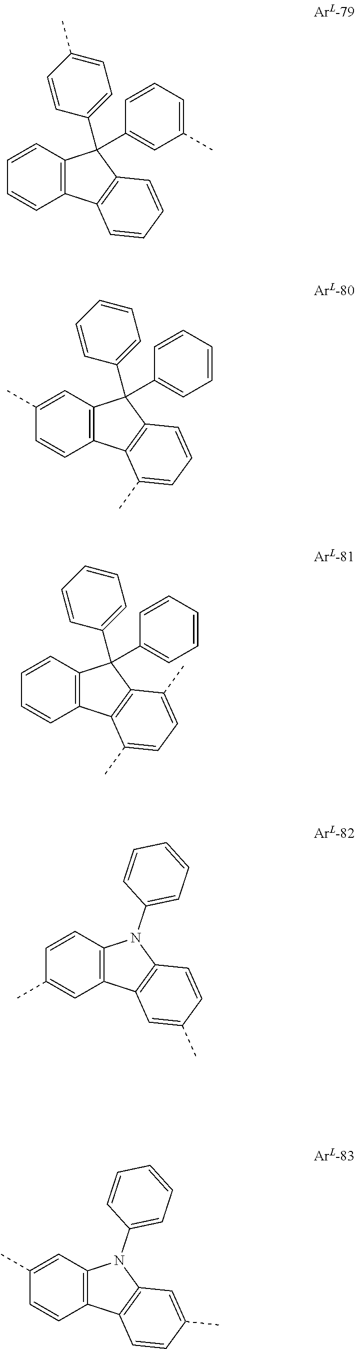

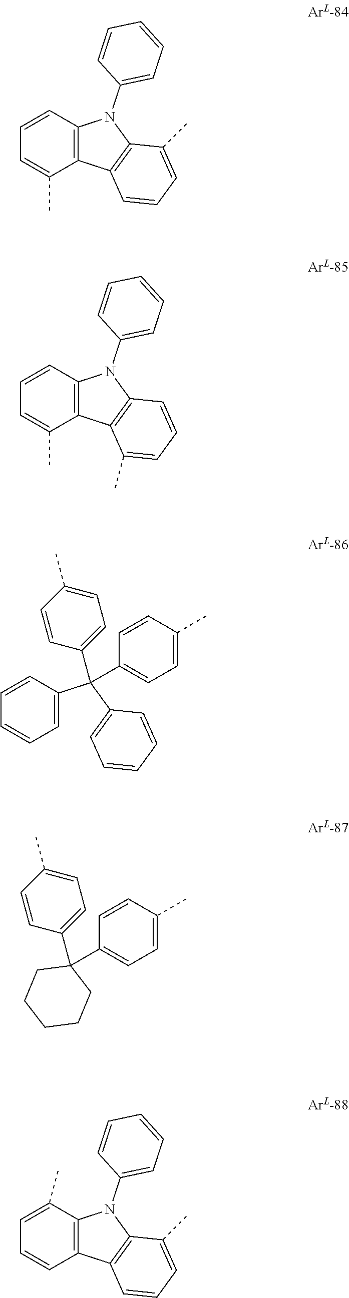

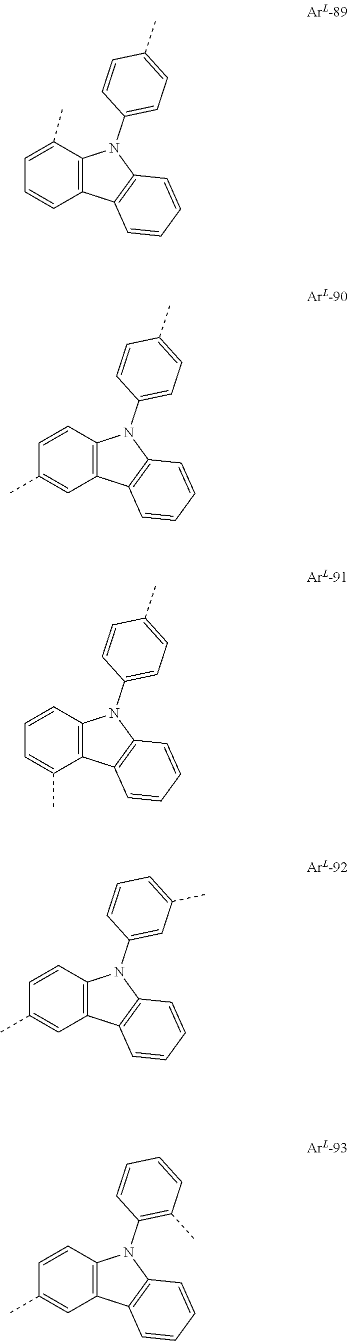

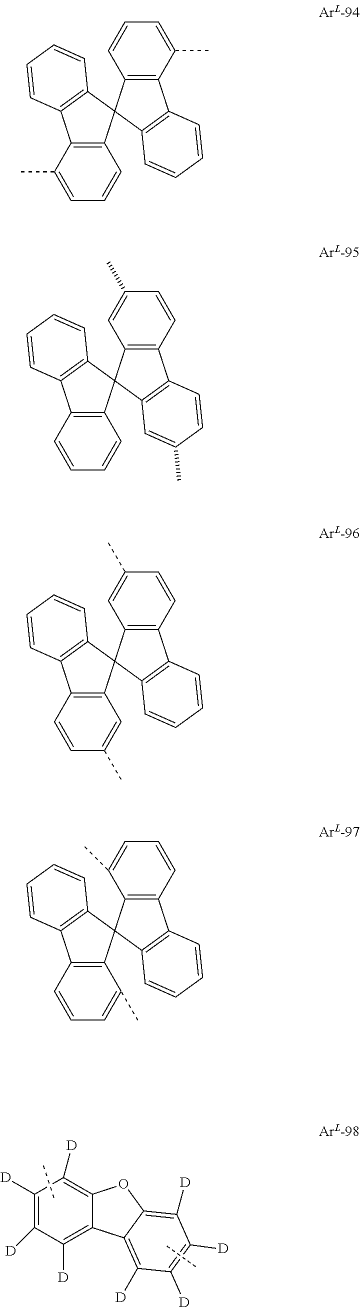

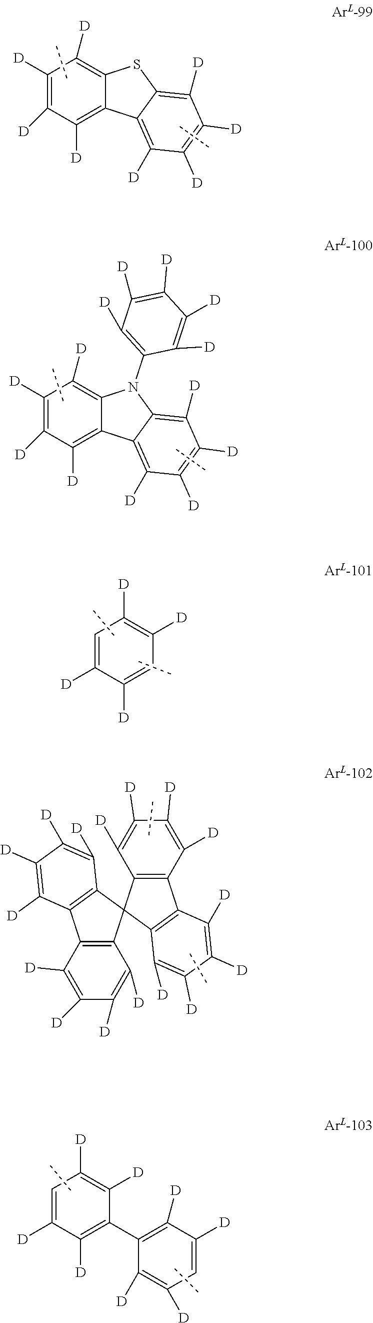

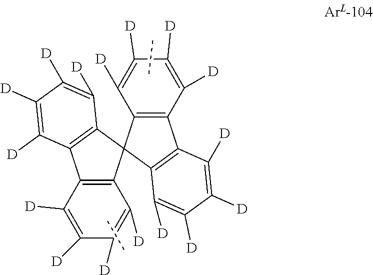

which are each substituted by R³ radicals at the positions shown as unsubstituted, where R³ in these cases is preferably the same or different and is selected from H and D and is more preferably H. Among the abovementioned formulae for Ar^{L}, particular preference is given to the formulae Ar^{L}-23 to Ar^{L}-26, Ar^{L}-37, Ar^{L}-42, Ar^{L}-47, and Ar^{L}-58, very particular preference to the formulae Ar^{L}-23 to Ar^{L}-25.

In a preferred embodiment, index n is 0, and so formula (I) conforms to the preferred formula (I-A). In an alternative preferred embodiment, index n is 1, and so formula (I) conforms to the preferred formula (I-B), more preferably to formula (I-B-1):

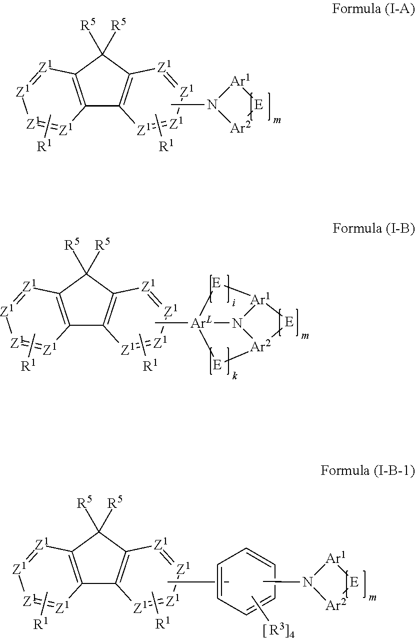

where the groups that occur are as defined above and preferably correspond to their preferred embodiments, and where R³ in formula (I-B-1) is preferably H.

Preferred embodiments of the formula (I-B-1) conform to the formulae (I-B-1-1) and (I-B-1-2)

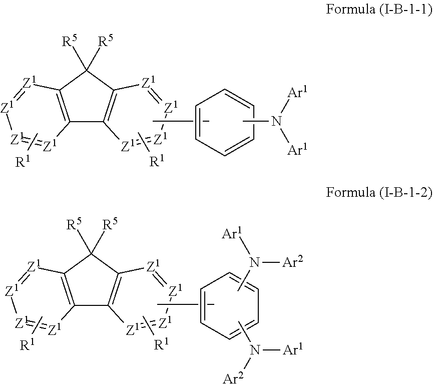

where the variables that occur are as defined above, and preferably correspond to their preferred embodiments.

Preferred Ar¹ and Ar² groups are the same or different at each instance and are selected from the radicals benzene, biphenyl, terphenyl, quaterphenyl, naphthyl, fluorenyl, especially 9,9′-dimethylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where said radicals are each substituted by R⁴ radicals.

Particularly preferred Ar¹ and Ar² groups are the same or different at each instance and are selected from the radicals biphenyl, terphenyl, quaterphenyl, naphthyl, spirobifluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, spirobifluorenyl, dibenzofuranyl, dibenzothiophenyl, carbazolyl, pyridyl, pyrimidyl and triazinyl, where the abovementioned radicals are each substituted by R⁴ radicals.

Particularly preferred Ar¹ and Ar² groups are the same or different at each instance and are selected from the following groups:

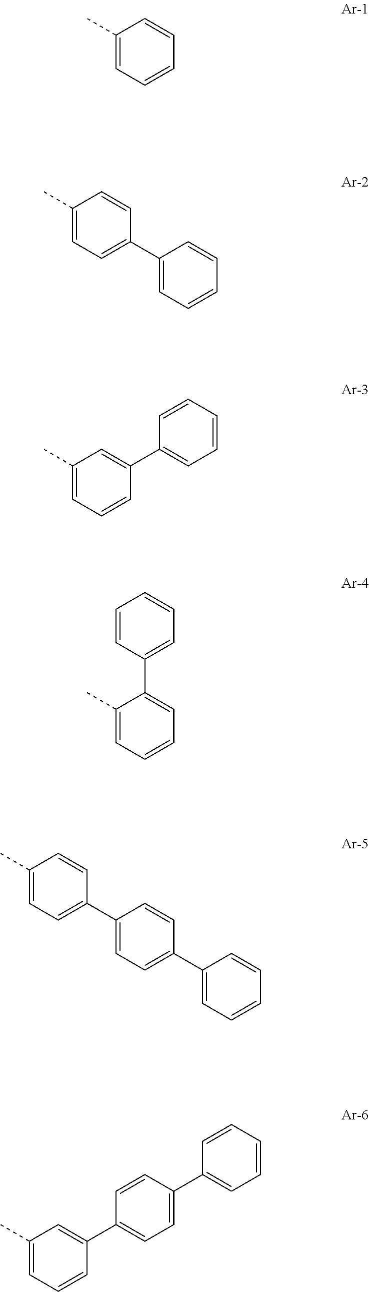

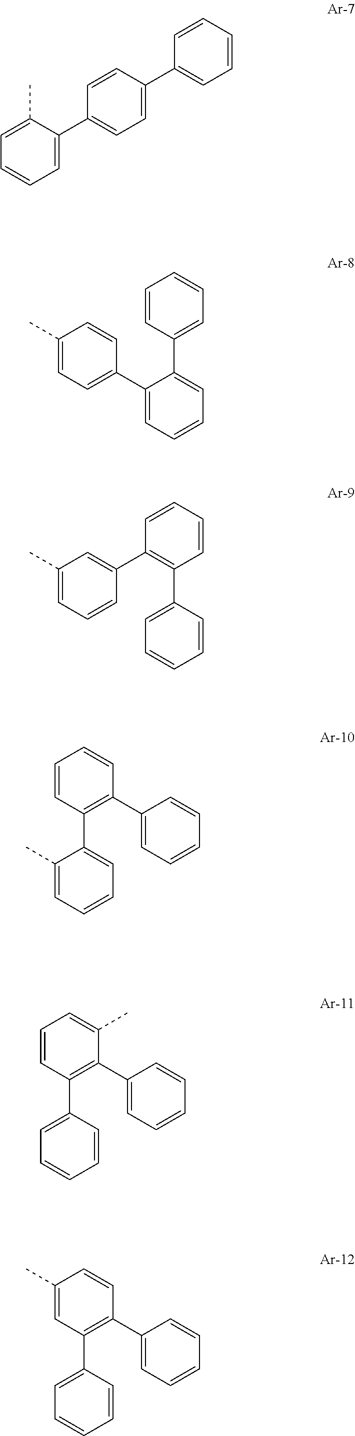

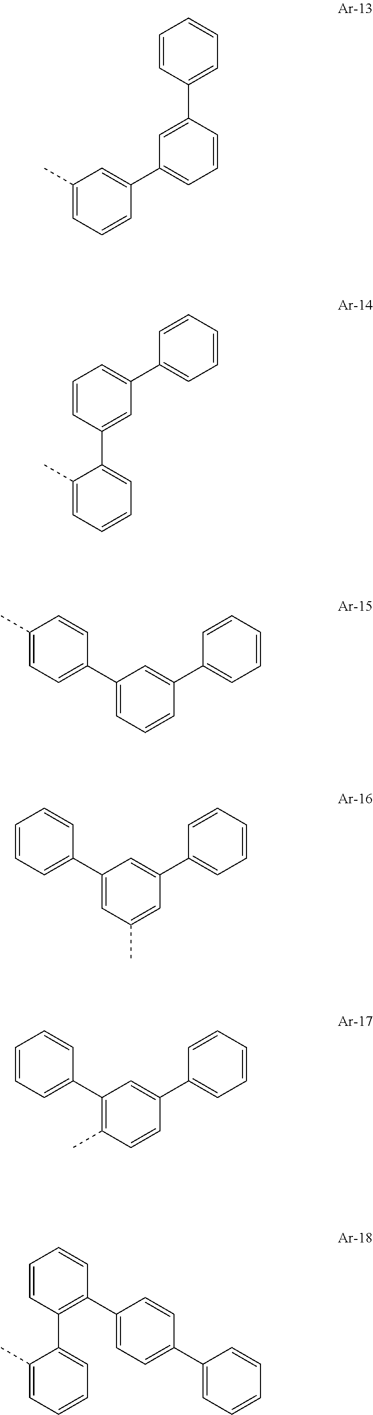

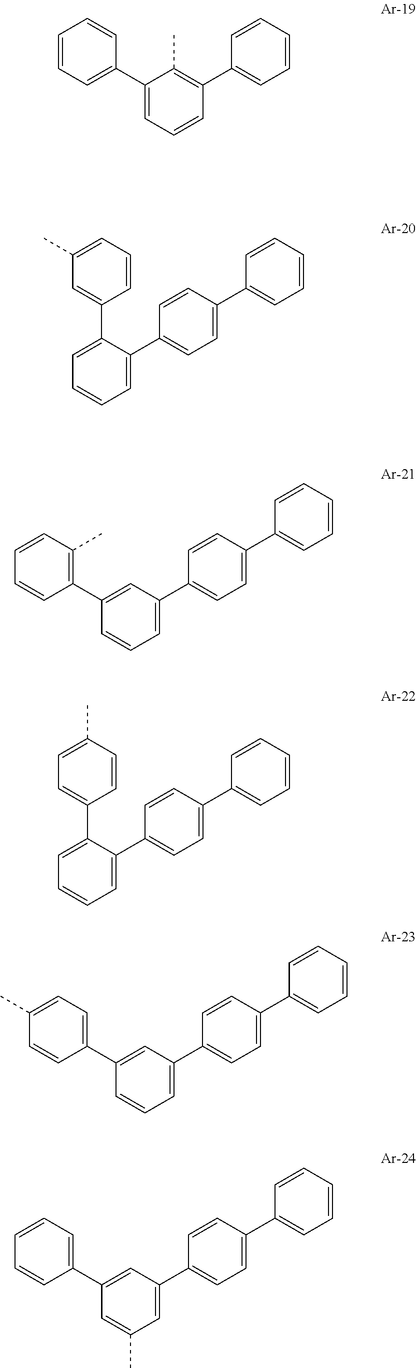

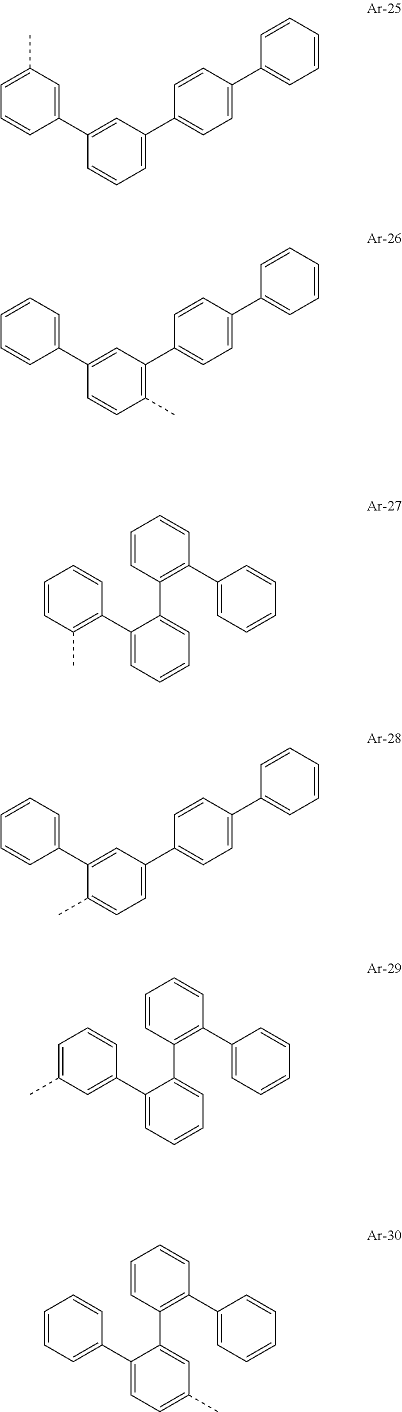

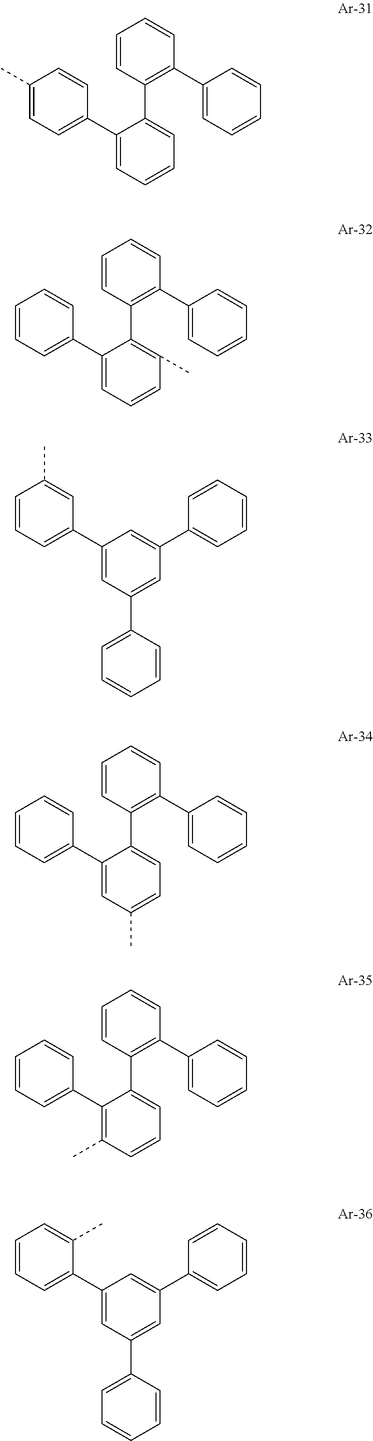

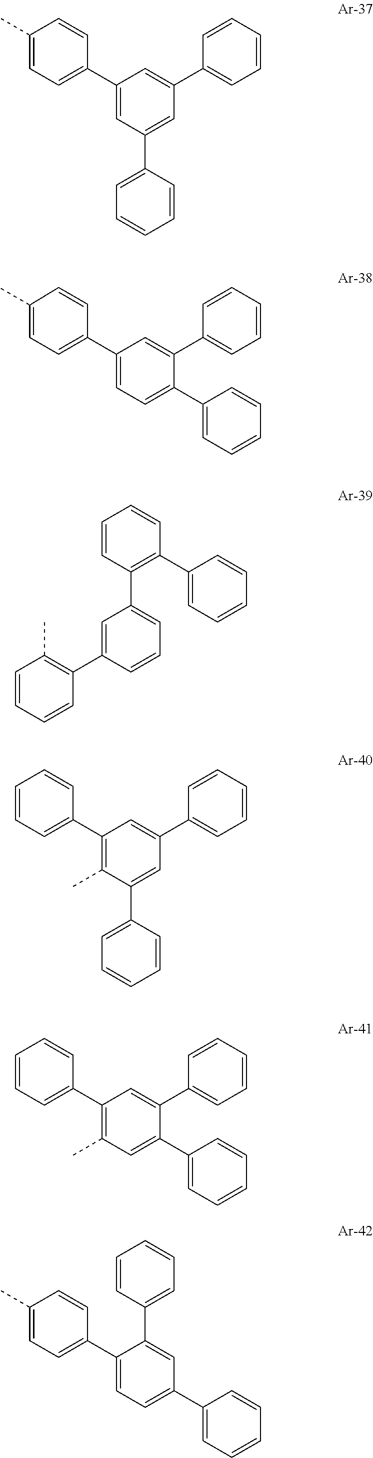

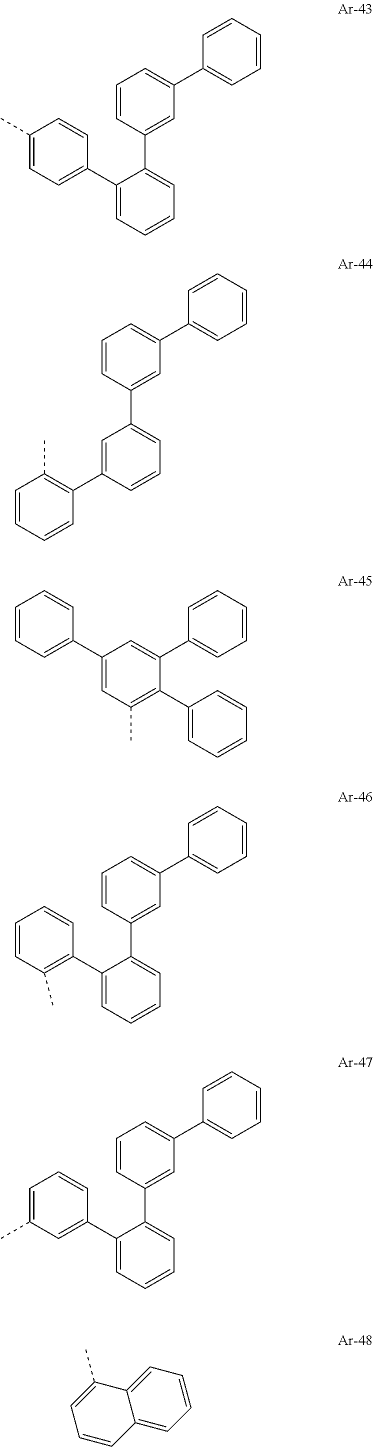

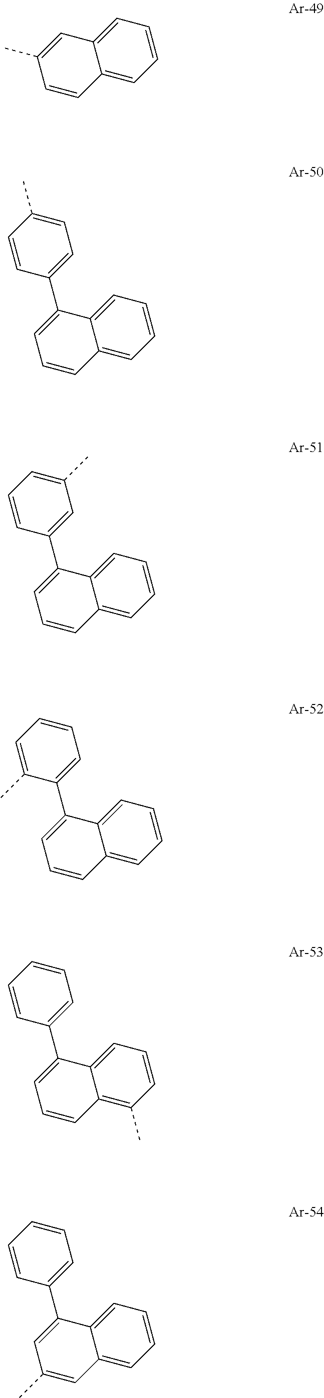

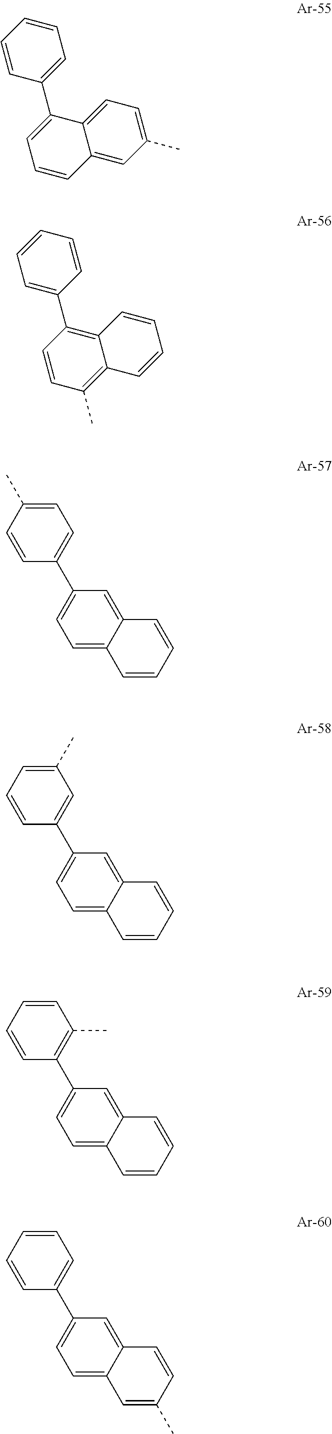

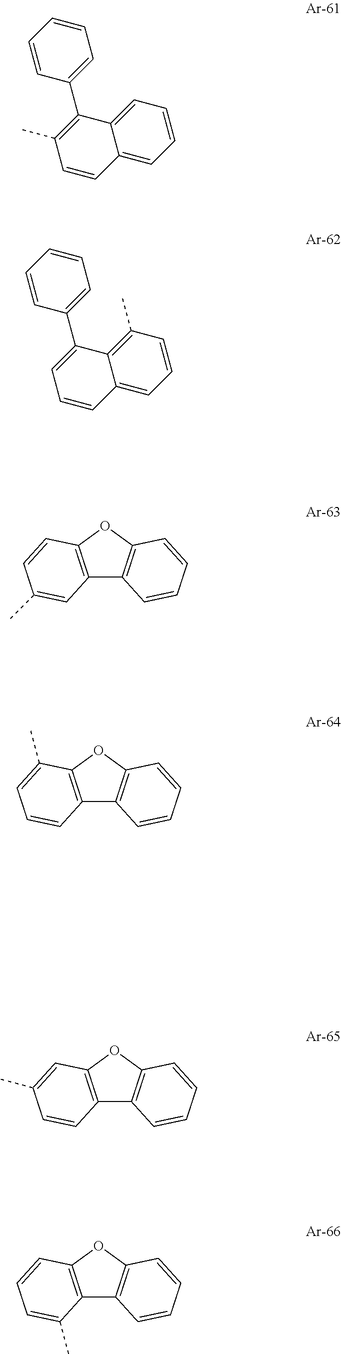

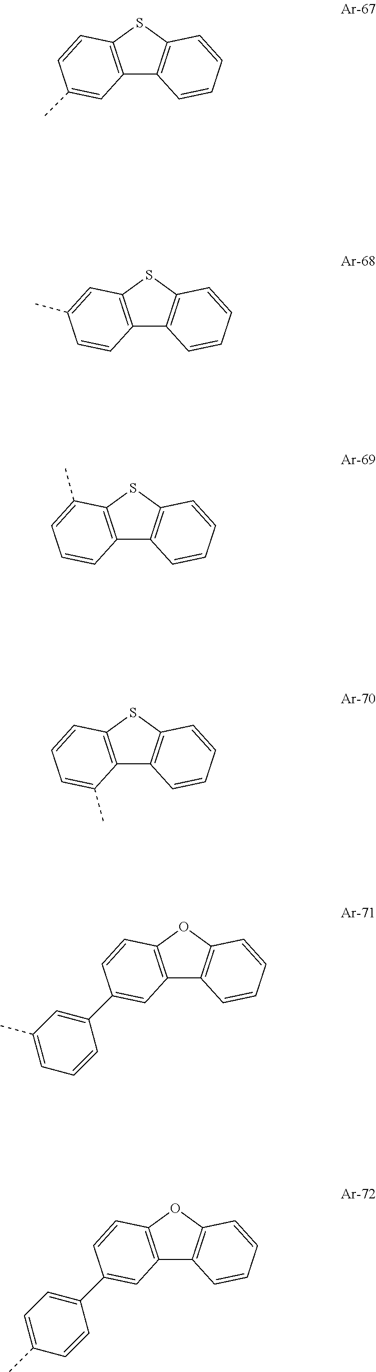

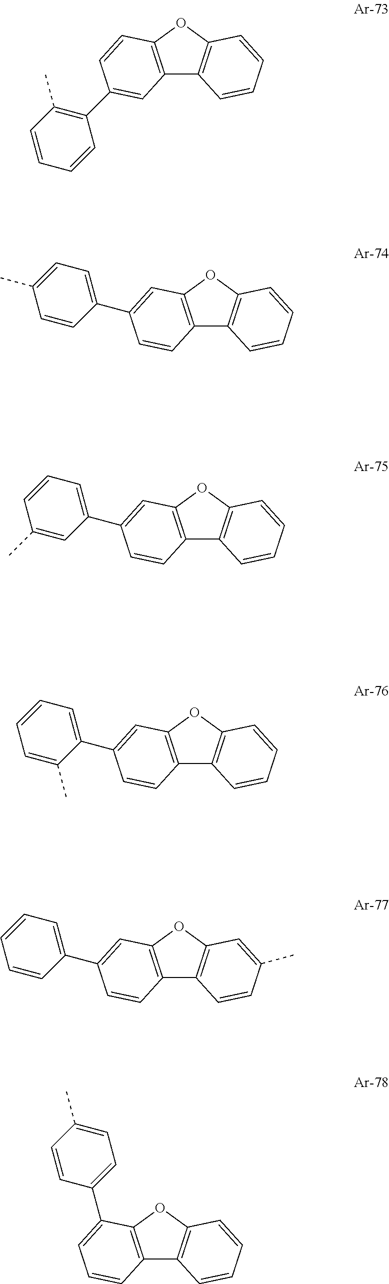

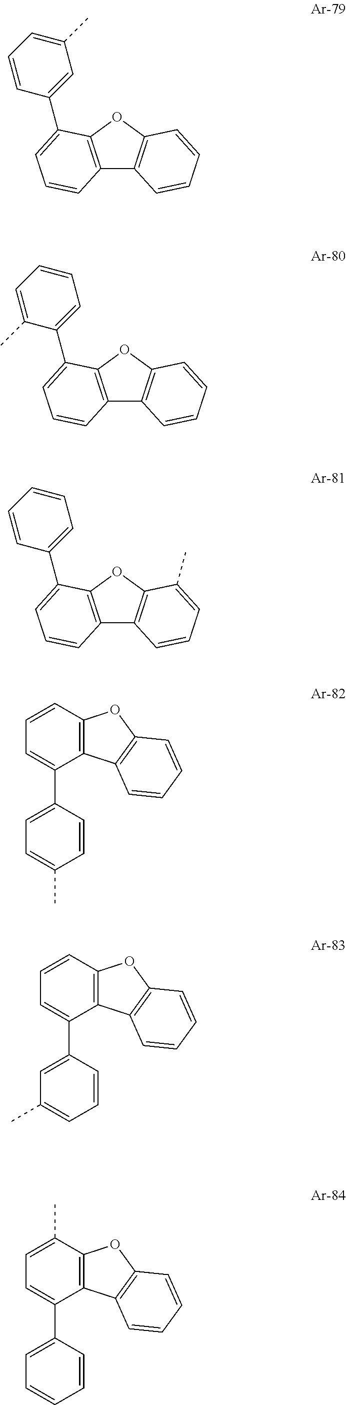

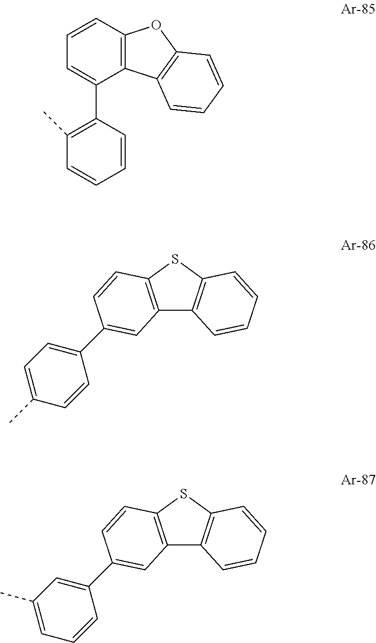

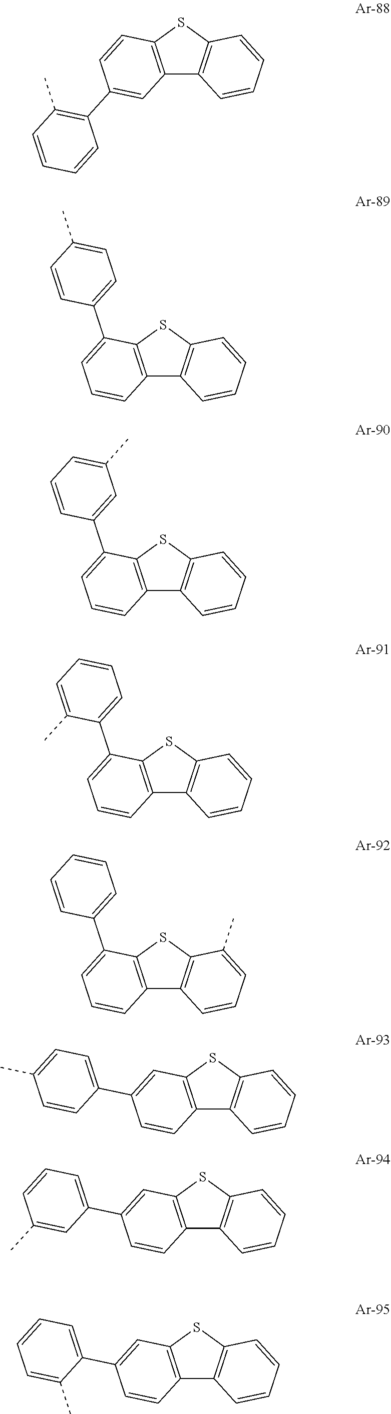

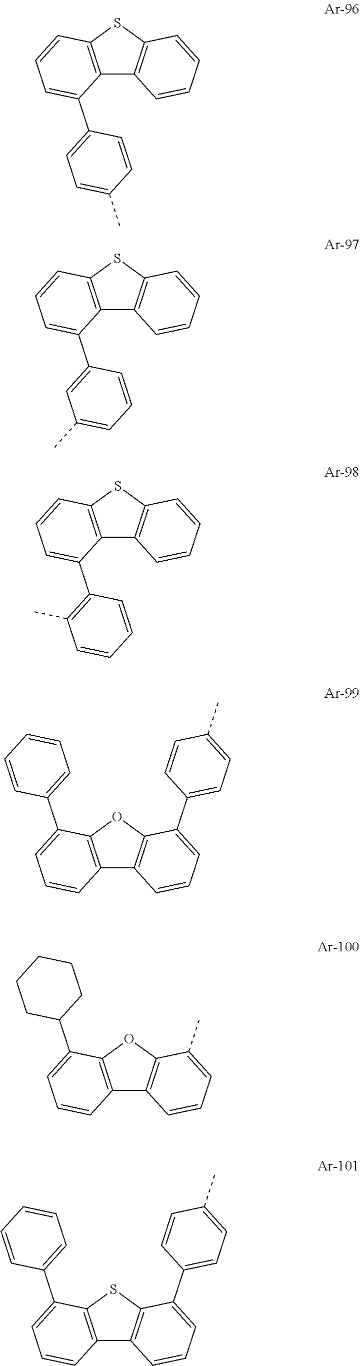

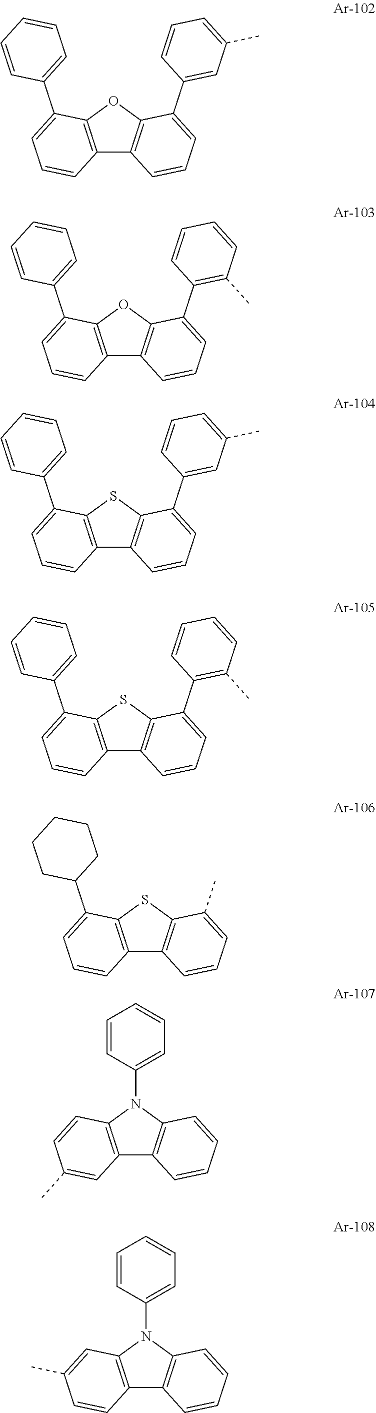

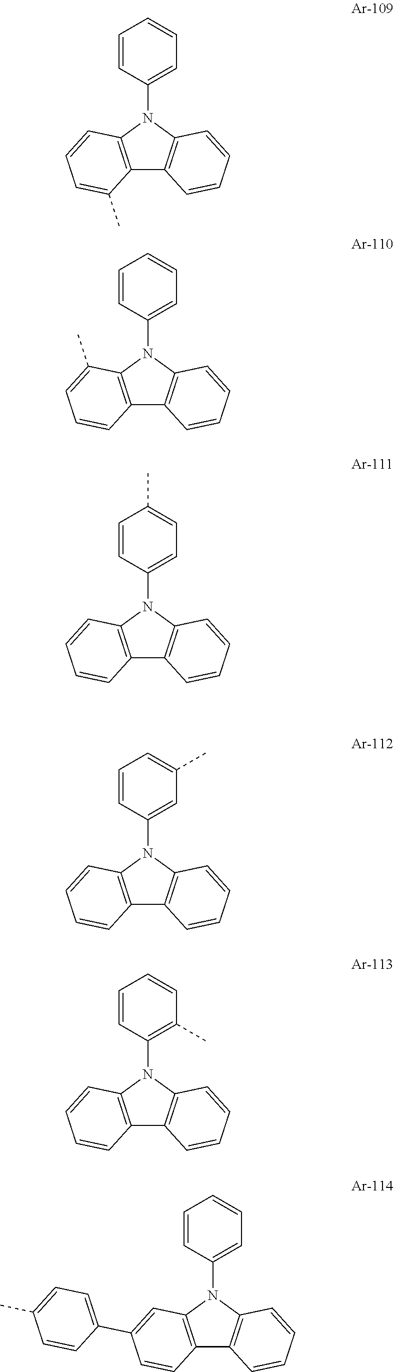

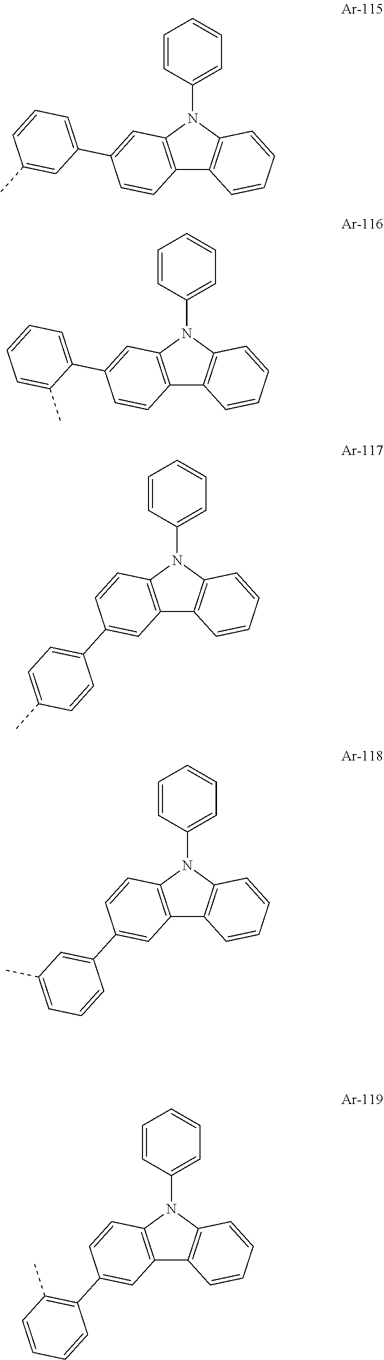

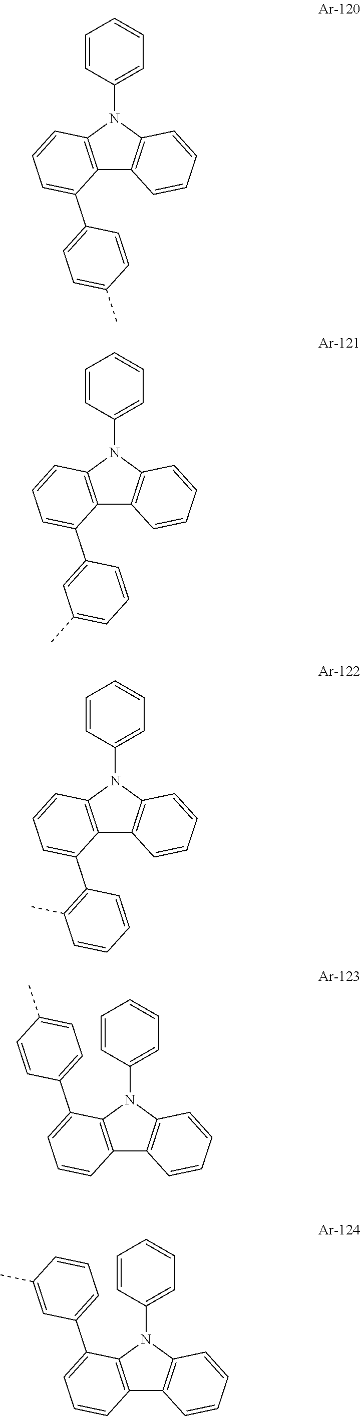

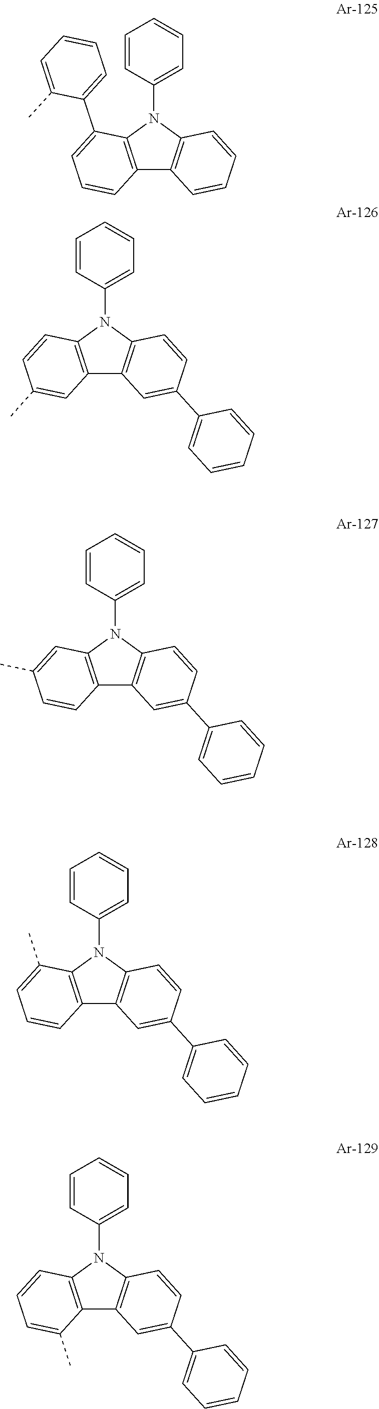

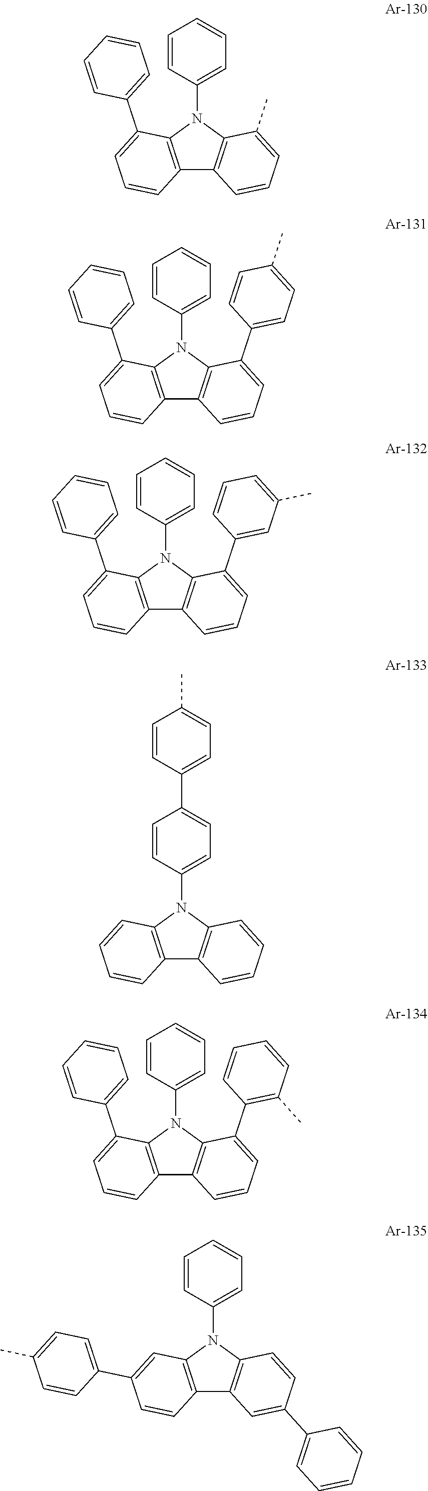

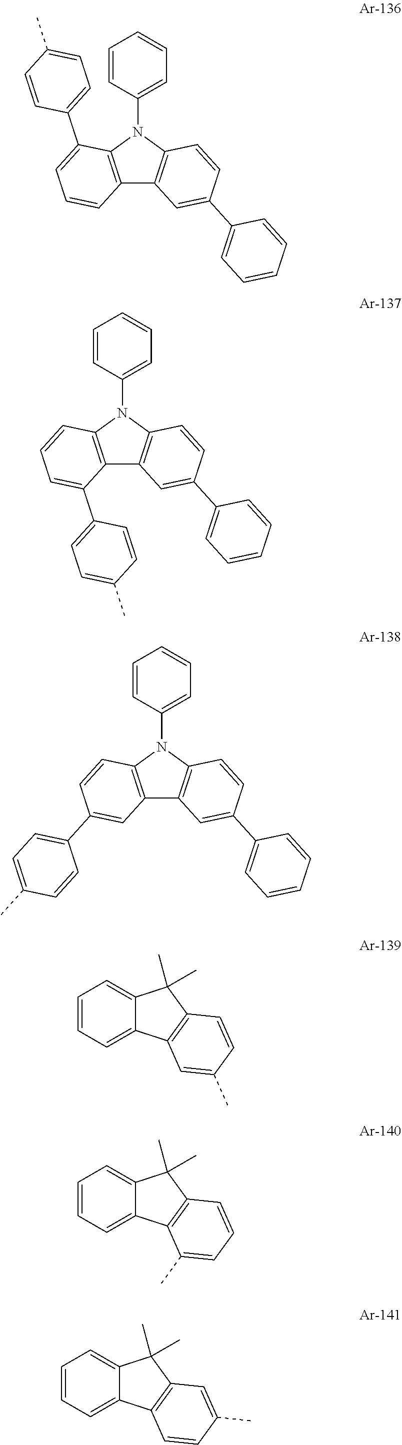

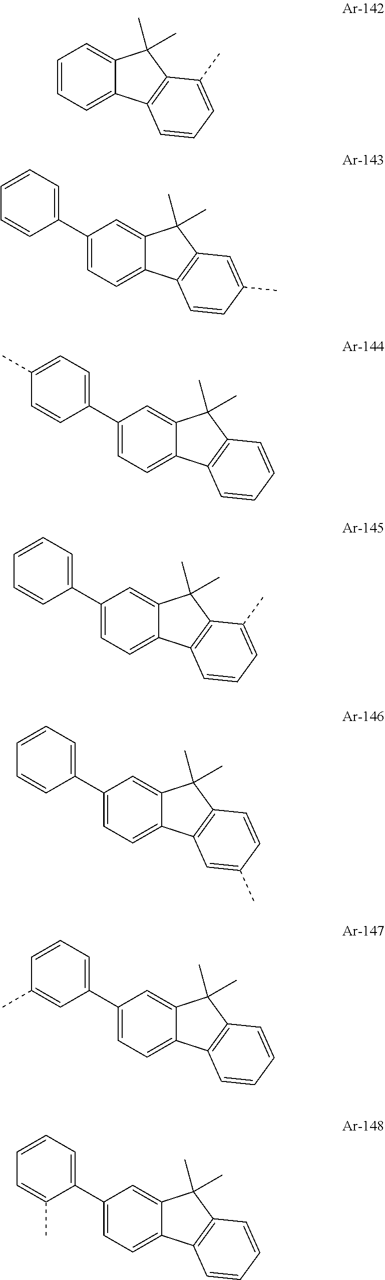

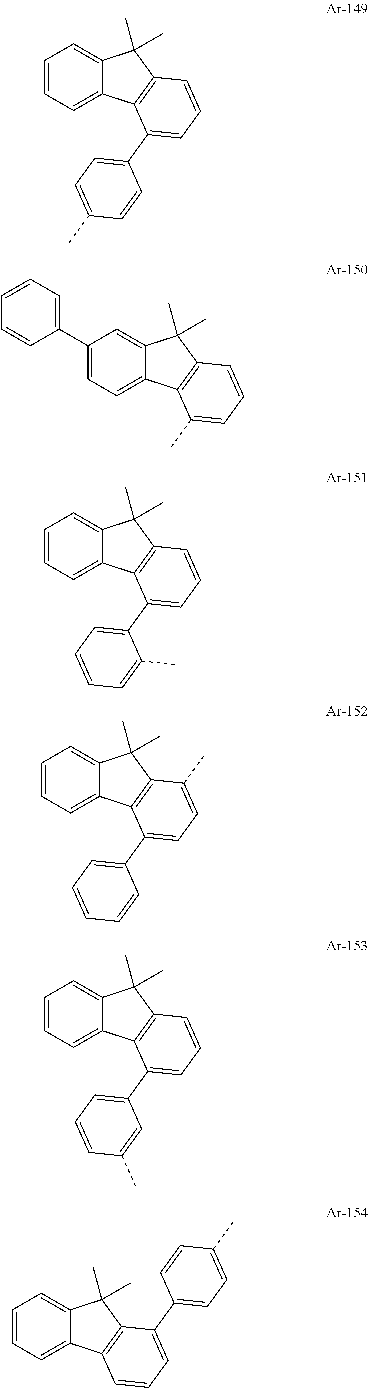

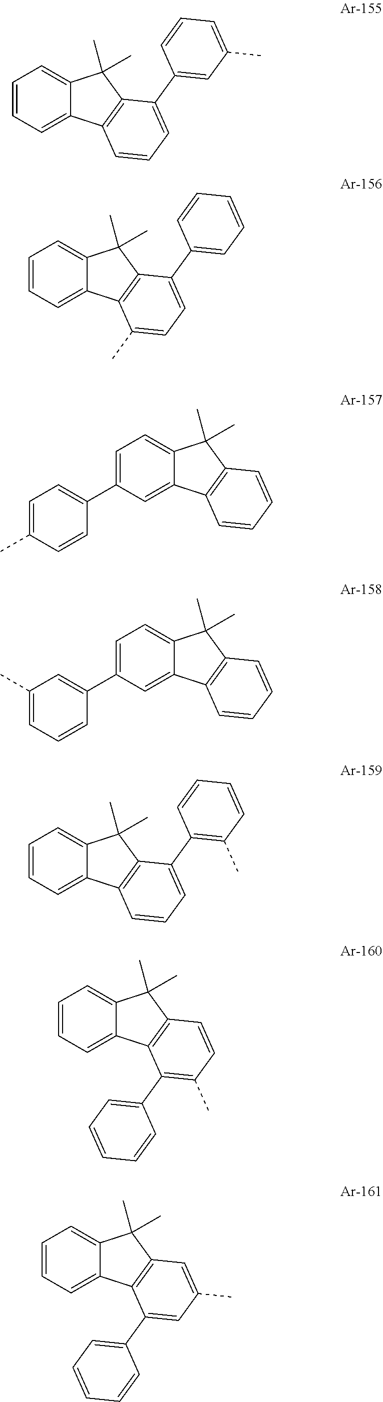

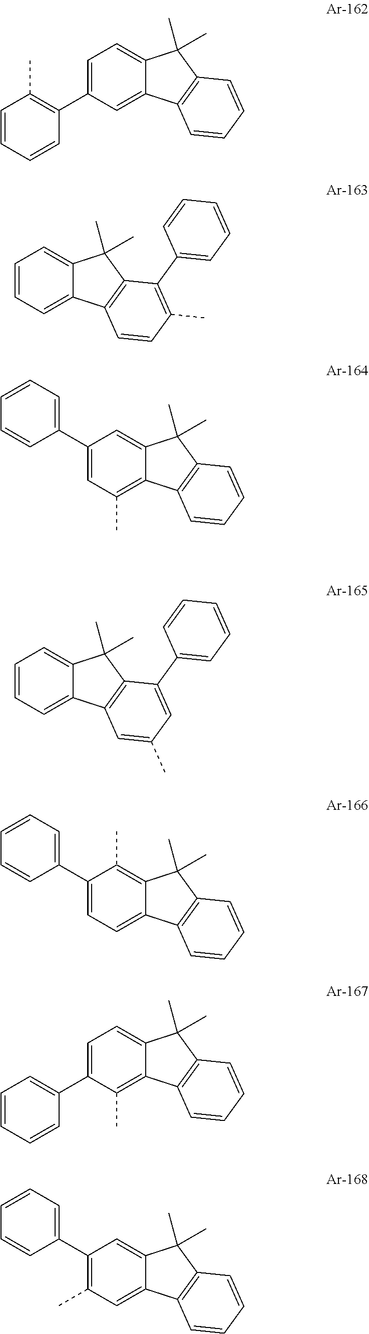

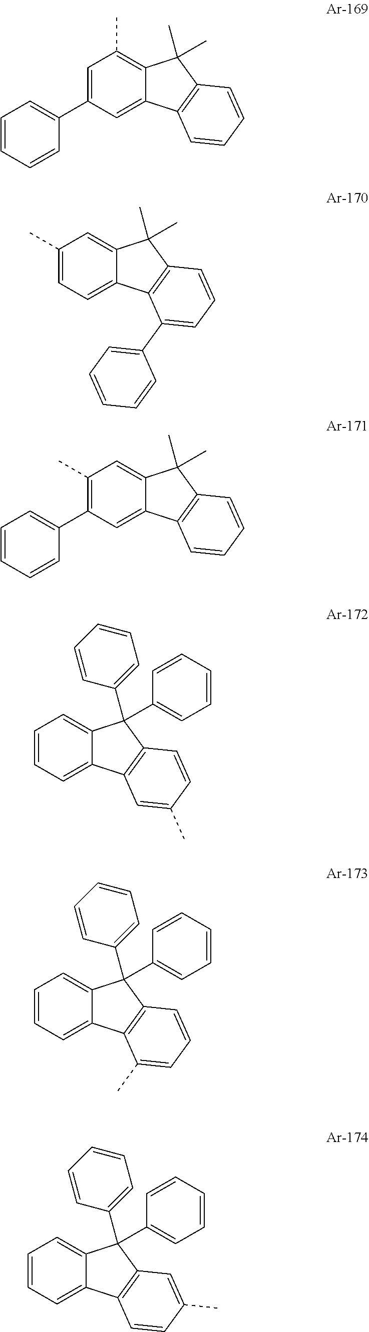

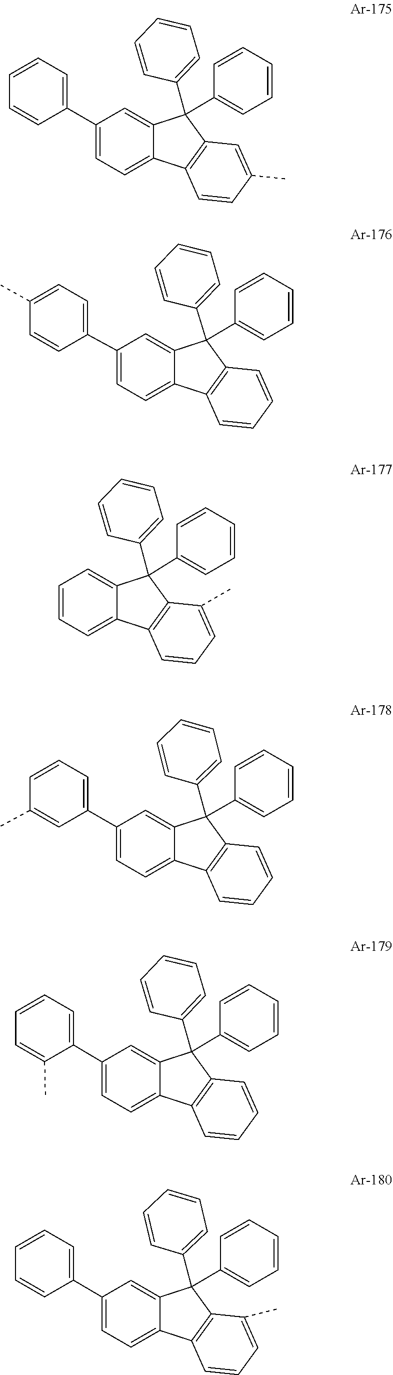

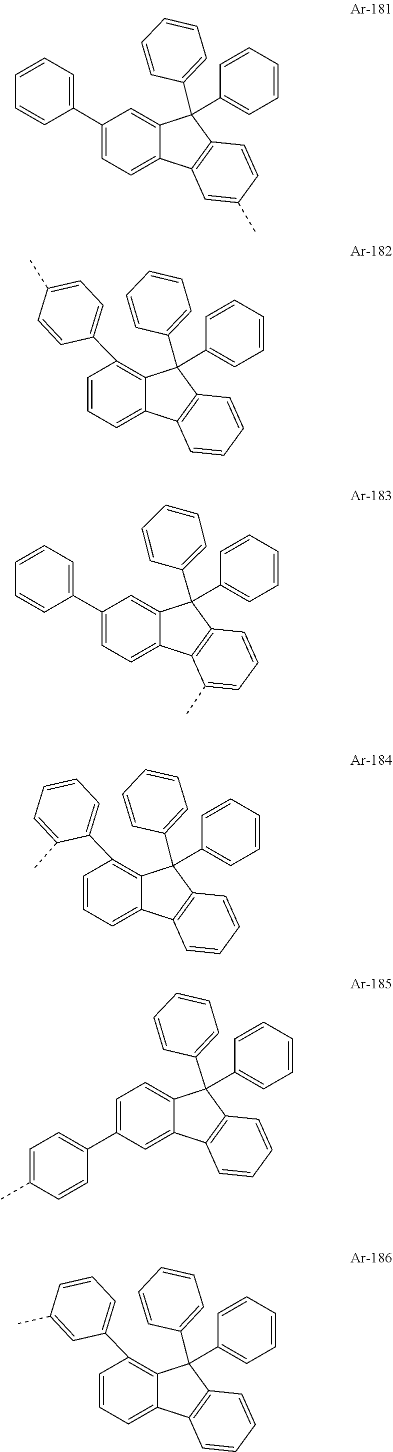

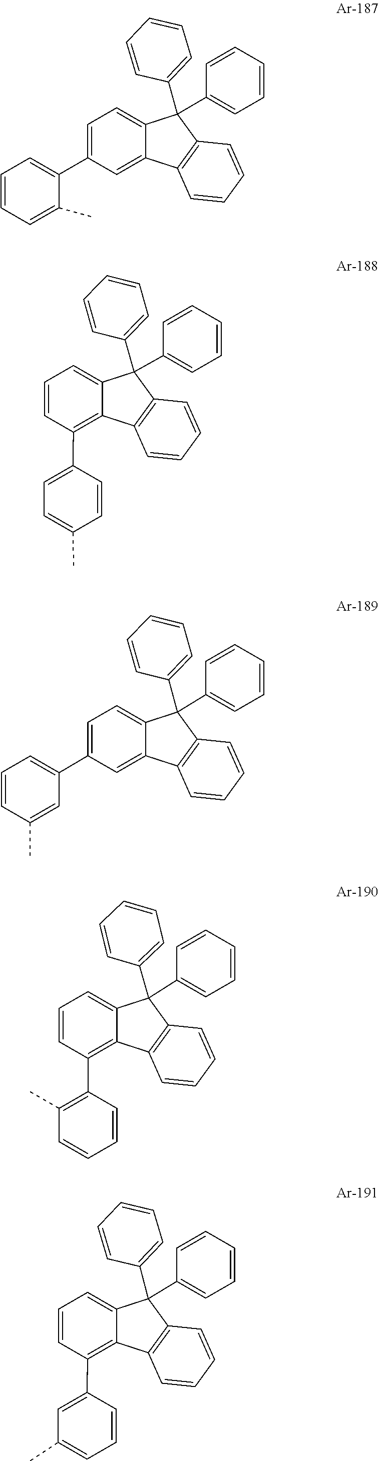

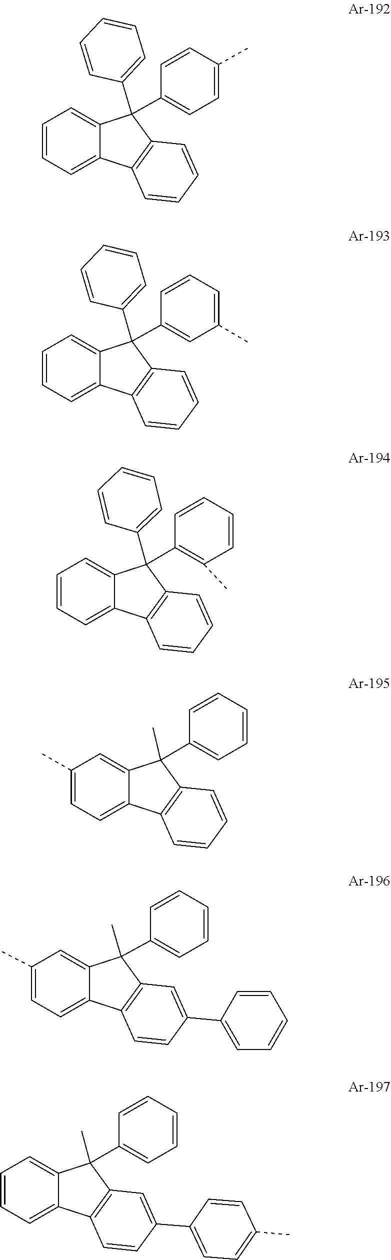

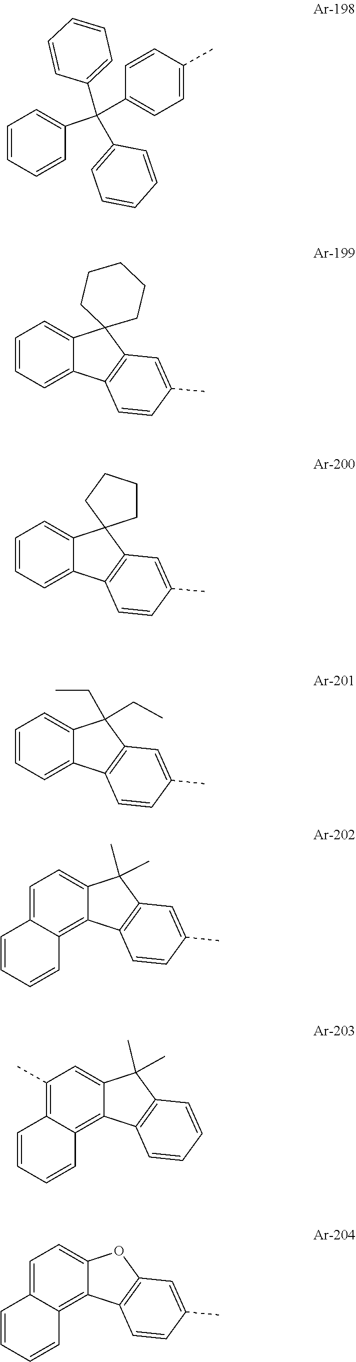

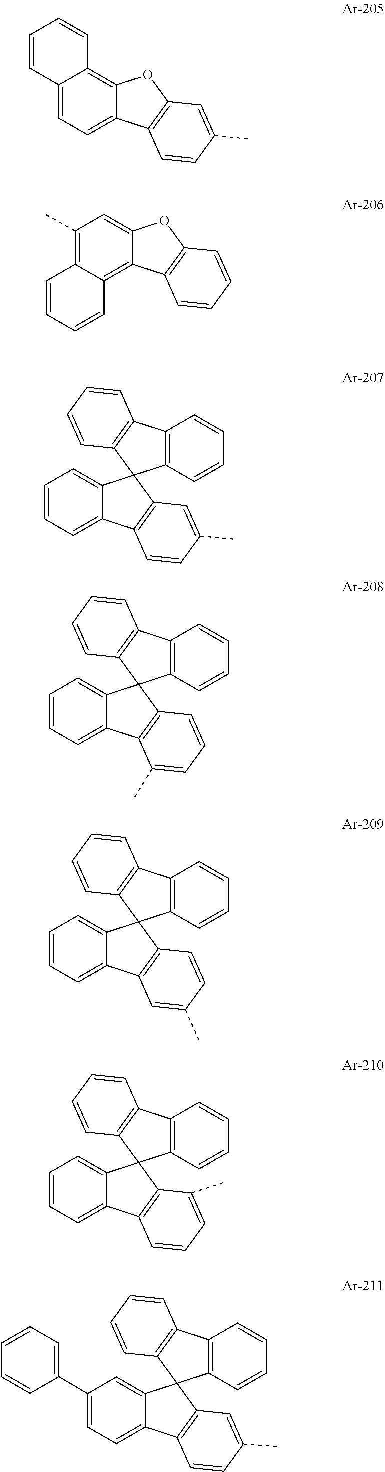

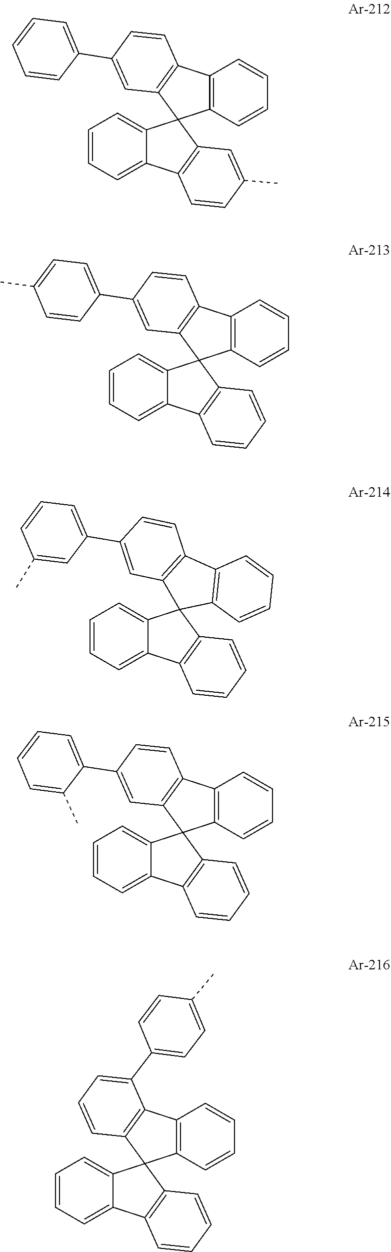

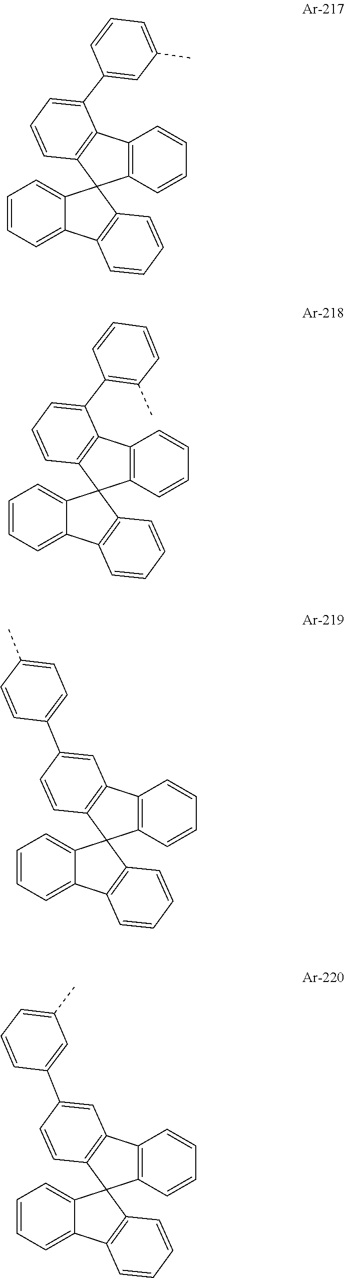

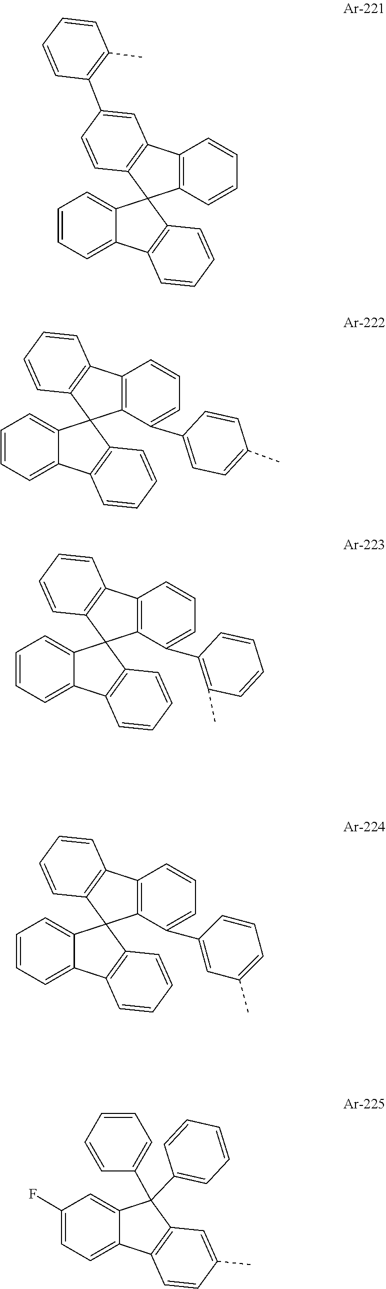

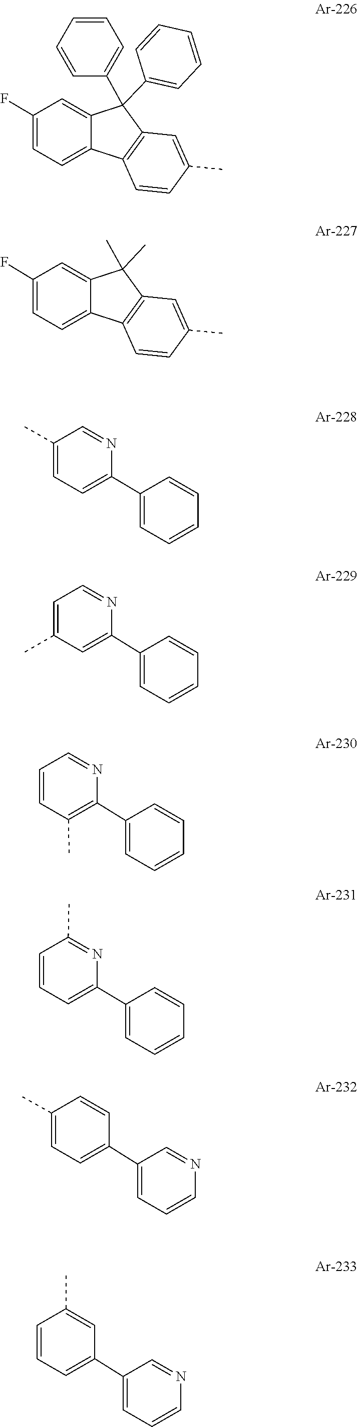

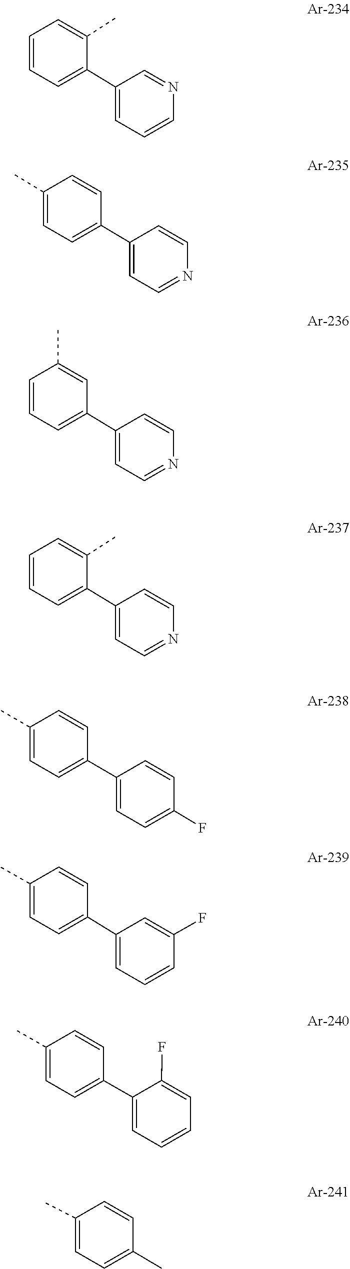

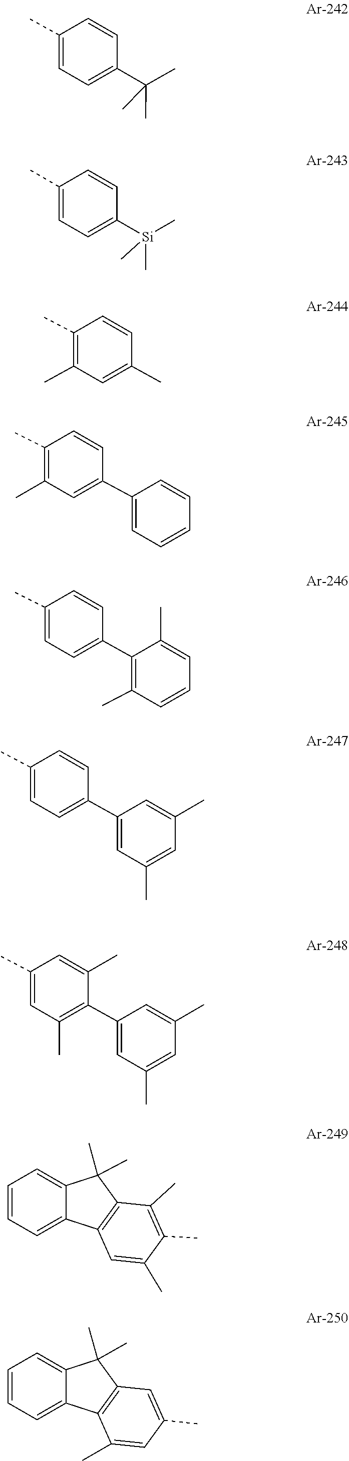

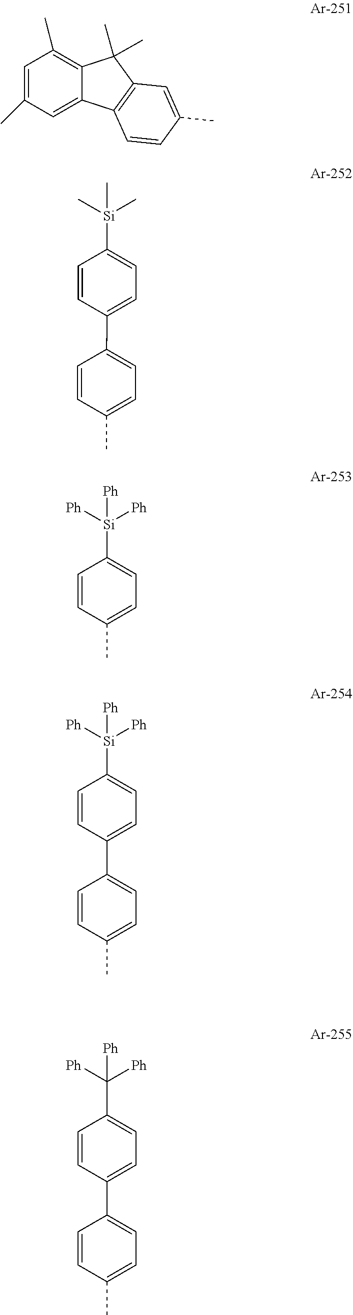

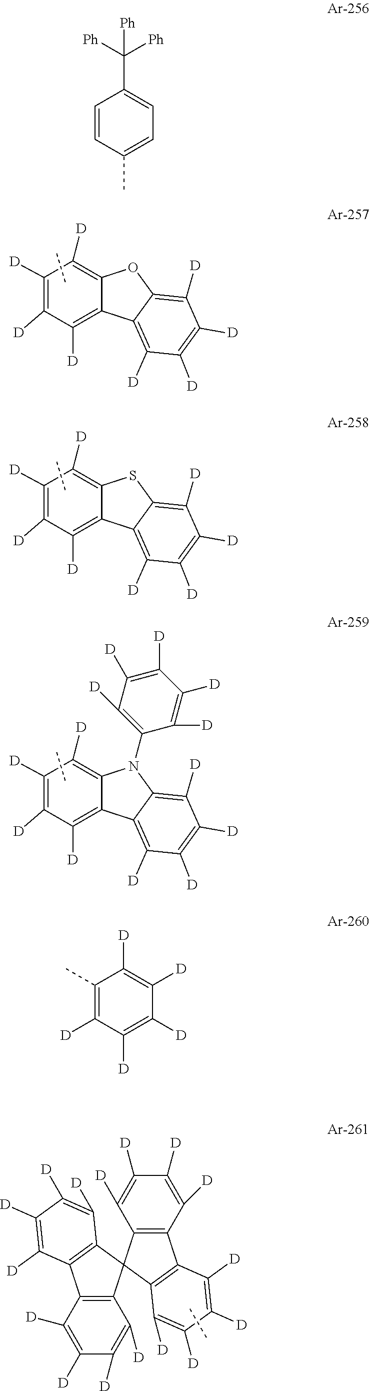

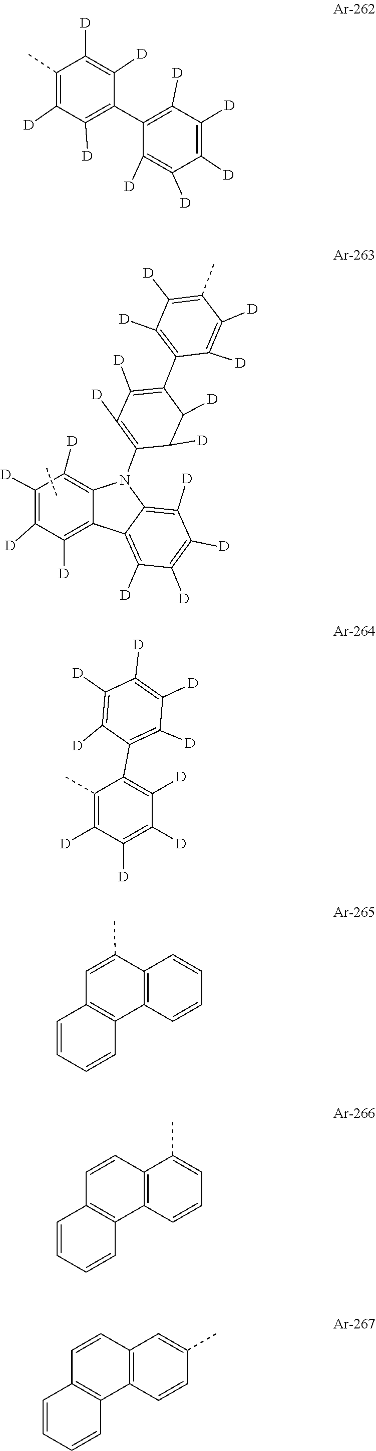

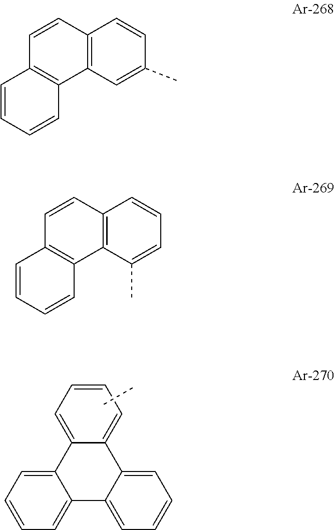

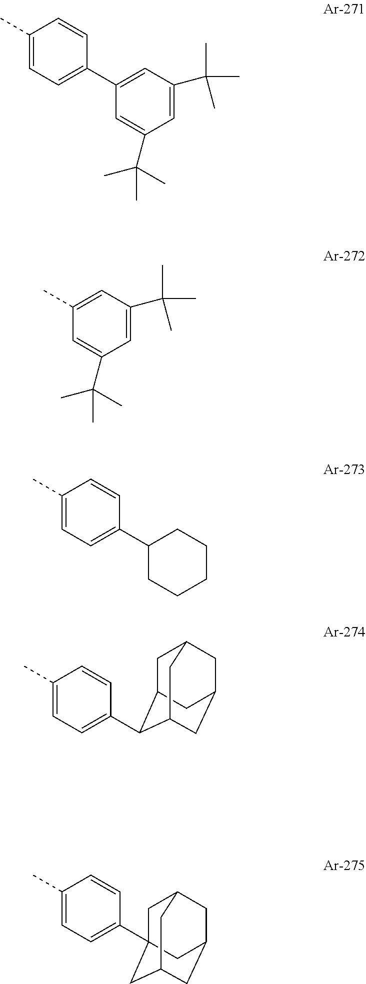

which are each substituted by R⁴ radicals at the positions shown as unsubstituted, where the R⁴ radicals in these cases are preferably H or D, more preferably H. Among the abovementioned formulae, particular preference is given to the formulae Ar-1, Ar-2, Ar-3, Ar-5, Ar-48, Ar-50, Ar-56, Ar-78, Ar-82, Ar-109, Ar-111, Ar-114, Ar-117, Ar-140, Ar-141, Ar-149, Ar-257, Ar-261, Ar-262 and Ar-263.

In a preferred embodiment, at least one group selected from the Ar¹ and Ar² groups is identical to a formula selected from the formulae (Ar-A) and (Ar-B):

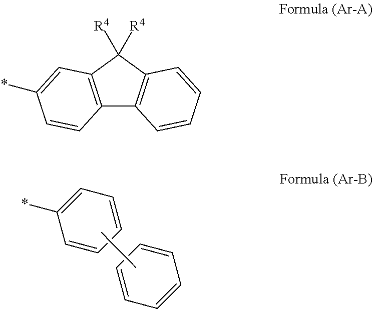

where the bond labeled * is the bond to the nitrogen atom of the formula (I), and where R⁴ in formula (Ar-A) is preferably the same or different at each instance and is selected from alkyl groups having 1 to 40 carbon atoms, more preferably from methyl, ethyl, propyl, butyl, each of which may be substituted by one or more fluorine atoms, especially from methyl which may be substituted by one or more fluorine atoms.

In a preferred embodiment, at least one group selected from the Ar¹ and Ar² groups is, preferably both groups selected from the Ar¹ and Ar² groups are, identical to the following formula (Ar-B):

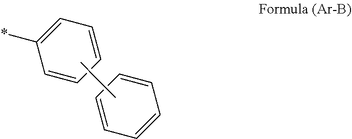

where the bond labeled * is the bond to the nitrogen atom of the formula (I).

In a particularly preferred embodiment, at least one group selected from the Ar¹ and Ar² groups is identical to a formula selected from formulae Ar-139 to Ar-152, Ar-172 to Ar-174 and Ar-177, preferably selected from formulae Ar-141 and Ar-174, where these are preferably unsubstituted on the benzene rings of the fluorenyl base skeleton, i.e. R⁴ is H.

In an alternative preferred embodiment, neither A^{r} nor Ar² is selected from fluorenyl groups; more preferably, neither A^{r} nor Ar² contains fluorenyl groups.

In a further preferred embodiment, at least one group selected from the Ar¹ and Ar² groups is a heteroaromatic ring system, especially a heteroaryl group, which has 5 to 40 aromatic ring atoms and is substituted by R⁴ radicals, especially a heteroaromatic ring system, in particular again a heteroaryl group, selected from the above-specified preferred embodiments for Ar¹ and Ar².

When a group selected from Ar¹ and Ar² groups is fluorenyl, it is preferable that the fluorenyl group is unsubstituted on its benzene rings.

It is further preferable that both groups selected from Ar¹ and Ar² are the same or different and are selected from biphenyl; terphenyl; quaterphenyl; phenanthrenyl; dibenzofuranyl; dibenzothiophenyl; carbazolyl; and phenyl substituted by at least one group selected from fluorenyl, naphthyl, phenanthrenyl, dibenzofuranyl, dibenzothiophenyl, and carbazolyl, where these radicals are each substituted by R⁴ radicals.

It is further preferable that the at least one group selected from Ar¹ and Ar² is selected from biphenyl, terphenyl, quaterphenyl, phenanthrenyl, dibenzofuranyl, dibenzothiophenyl, and carbazolyl, where the groups mentioned are each substituted by R⁴ radicals, most preferably from dibenzofuranyl, dibenzothiophenyl, and carbazolyl, where the groups mentioned are each substituted by R⁴ radicals. More preferably, both groups Ar¹ and Ar² are the same or different and are selected from biphenyl, terphenyl, quaterphenyl, phenanthrenyl, dibenzofuranyl, dibenzothiophenyl, and carbazolyl, where the groups mentioned are each substituted by R⁴ radicals, most preferably from dibenzofuranyl, dibenzothiophenyl, and carbazolyl, where the groups mentioned are each substituted by R⁴ radicals.

In a preferred embodiment, Ar¹ and Ar² are different. In this case, the three groups bonded to the nitrogen atom in formula (I) are different, where groups are understood to mean not just the groups bonded directly to the nitrogen atom but the complete groups including their possible substituents.

What is meant by “different” is not just that the groups have different empirical formulae, where the term “empirical formula” in this case also includes H and D as different atoms, but also that they are different isomers, as is the case, for example, for o-biphenyl and p-biphenyl.

Preferred embodiments of the formula (I) conform to the following formula (I-1):

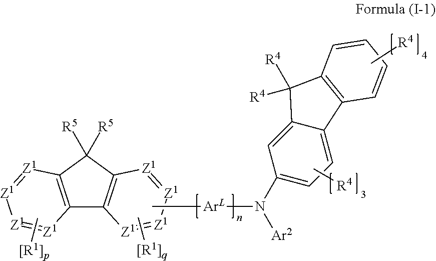

where the groups and indices that occur are as defined above and preferably conform to their preferred embodiments specified above, and where the —[Ar^{L}]ₙ—N group is bonded in the 1, 3 or 4 position of the fluorenyl group and where Ar² is selected from the following radicals: biphenyl; terphenyl; quaterphenyl; phenanthrenyl; dibenzofuranyl; dibenzothiophenyl; carbazolyl; and phenyl, which is substituted by at least one group selected from fluorenyl, naphthyl, phenanthrenyl, dibenzofuranyl, dibenzothiophenyl, and carbazolyl, where these radicals are each substituted by R⁴ radicals.

It is especially preferable for the formula (1-1) that the R⁴ groups on the benzene rings of the fluorenyl groups are H. Compounds of this kind have better properties as OLED materials than compounds in which the benzene rings of the fluorenyl groups are substituted.

Furthermore, it is especially preferable that R⁴ at the bridgeheads of the fluorenyl groups as Ar¹ or Ar² is the same or different at each instance and is selected from straight-chain alkyl groups having 1 to 20 carbon atoms and branched alkyl groups having 3 to 20 carbon atoms, where the alkyl groups are substituted by R⁷ radicals, and R⁷ in these cases is preferably H, D or F, more preferably H.

In a preferred embodiment, it is a single bond.

It is preferable that i is 0. It is preferable that k is 0. It is preferable that m is 0. It is more preferable that i, k and m are 0.

In a preferred embodiment, R¹ is the same or different at each instance and is selected from straight-chain alkyl groups having 1 to 20 carbon atoms, branched or cyclic alkyl groups having 3 to 20 carbon atoms and aromatic ring systems having 6 to 40 aromatic ring atoms, where said alkyl groups and said aromatic ring systems are each substituted by R⁷ radicals, where R⁷ in this case is preferably H. More preferably, R¹ is the same or different at each instance and is selected from methyl, trifluoromethyl, tert-butyl and phenyl.

Preferably, R¹ is the same at each instance. In an alternative, likewise preferred embodiment, the two R¹ are selected differently.

Preferred embodiments of the formula (I) are shown below:

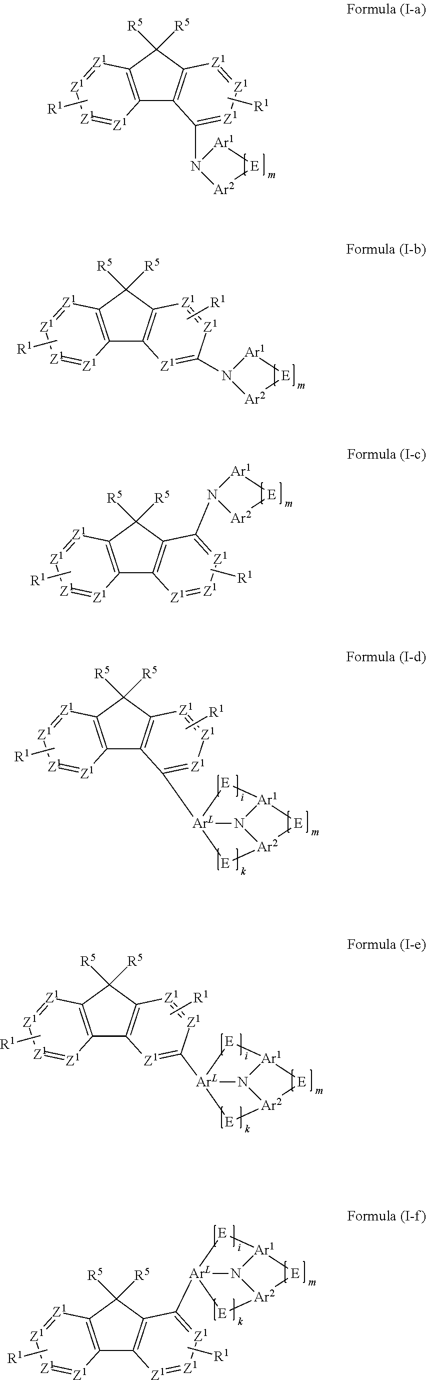

where the groups and indices that occur are as defined above and preferably conform to their preferred embodiments specified above. Among the formulae, the formulae (I-a) and (I-d) are particularly preferred.

In a particularly preferred embodiment, the compound conforms to the following formula (I-a-1)

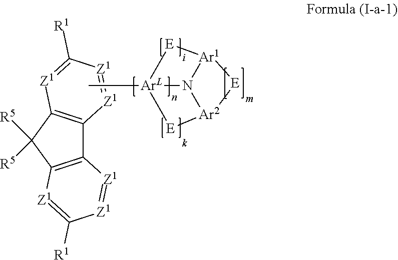

where the groups and indices that occur are as defined above and preferably conform to their preferred embodiments specified above, and where the —[Ar^{L}]ₙ—N group is bonded in the 1, 3 or 4 position of the fluorenyl group, preferably in the 4 position.

Preferred embodiments of the formula (I) also conform to the following formulae:

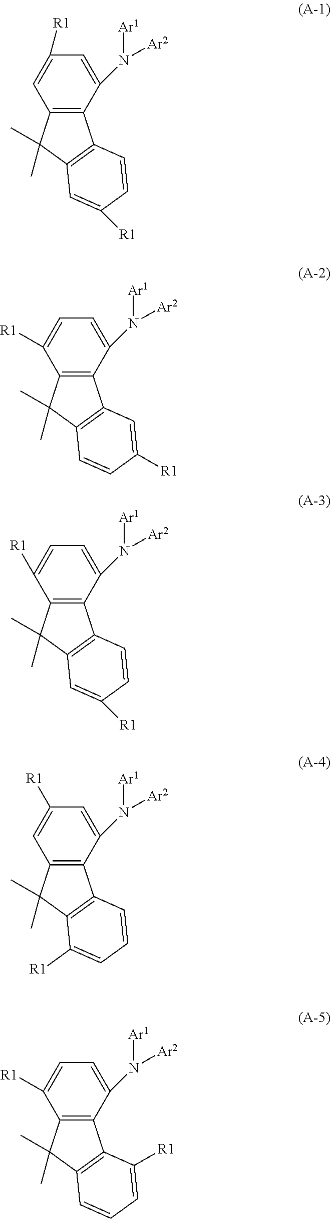

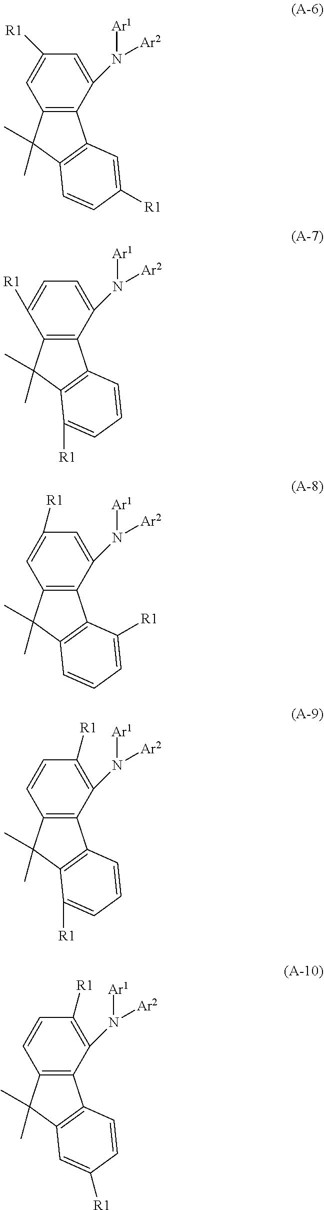

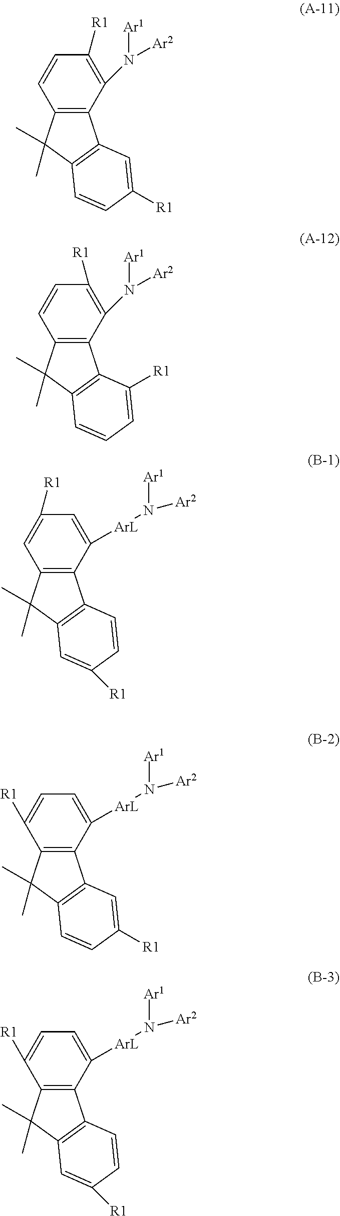

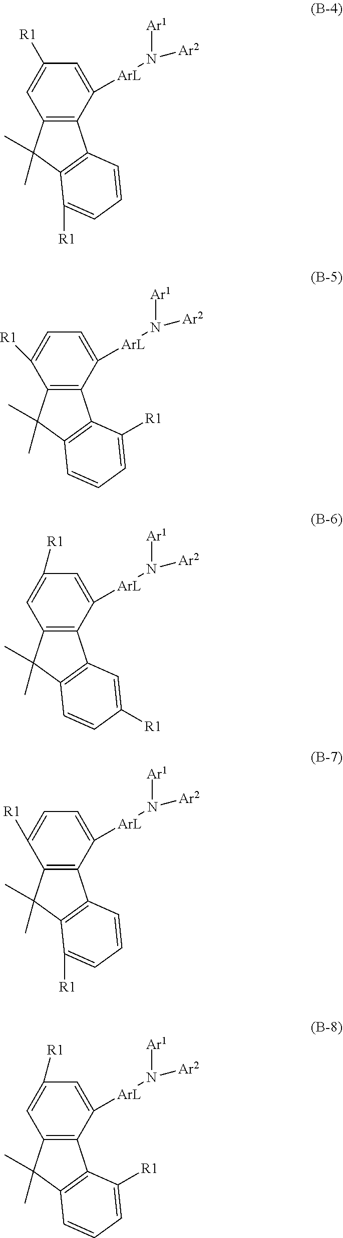

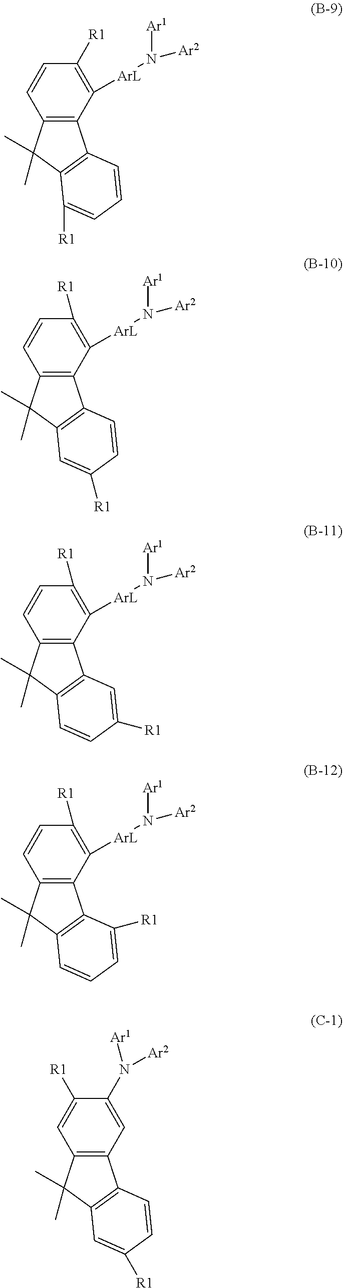

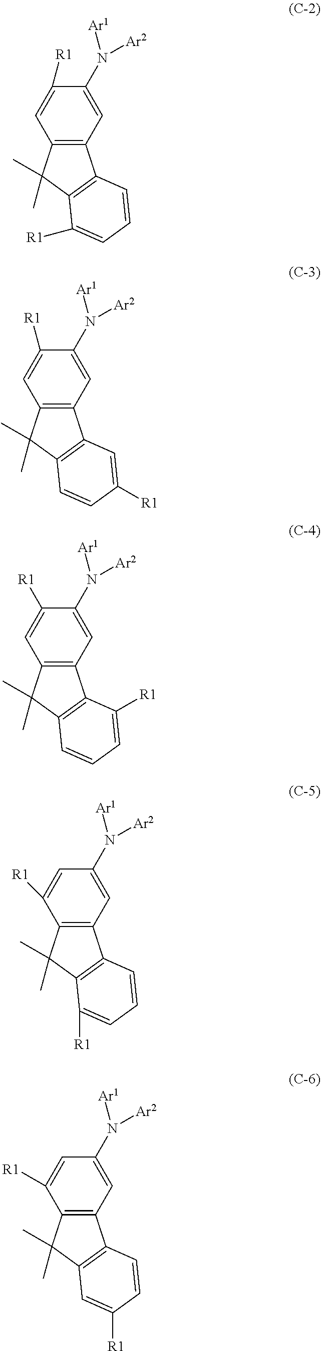

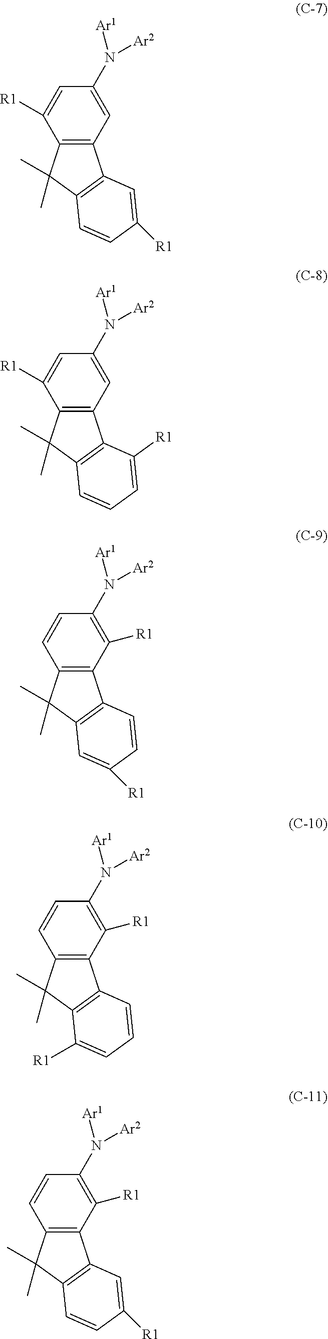

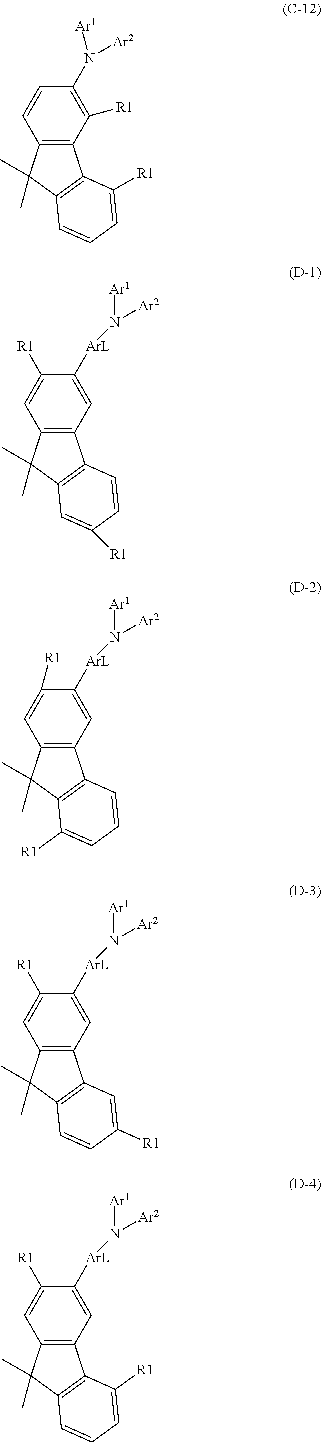

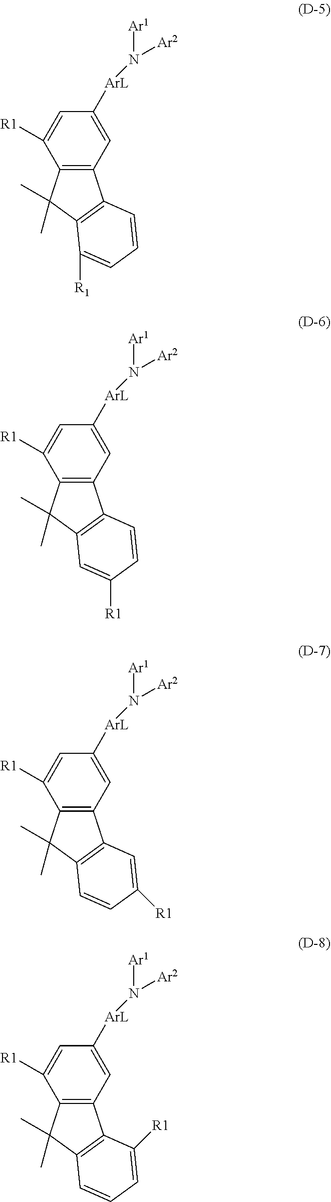

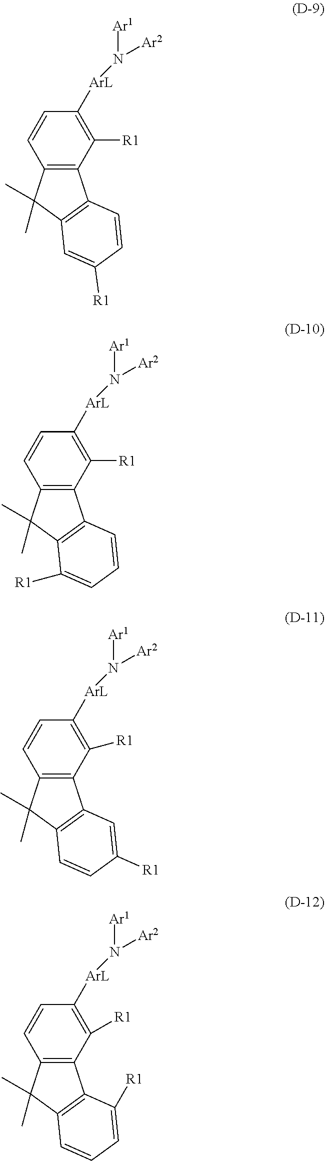

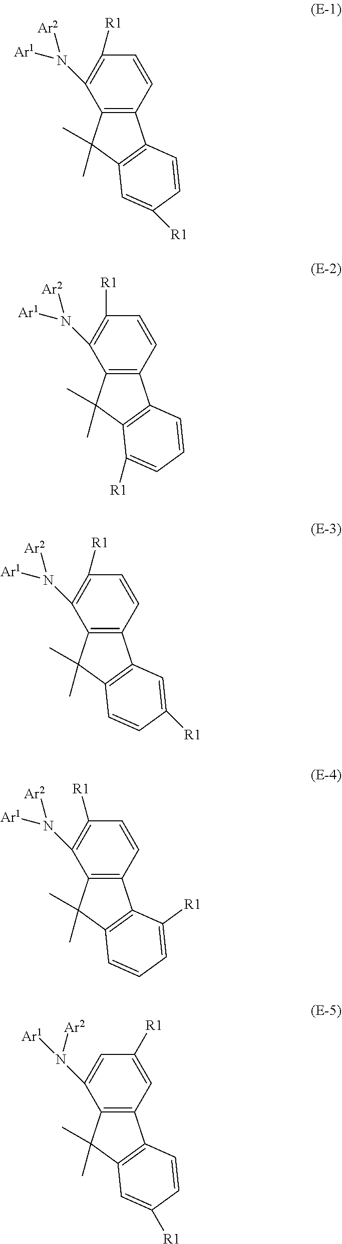

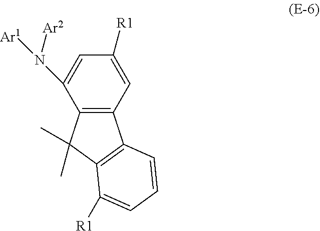

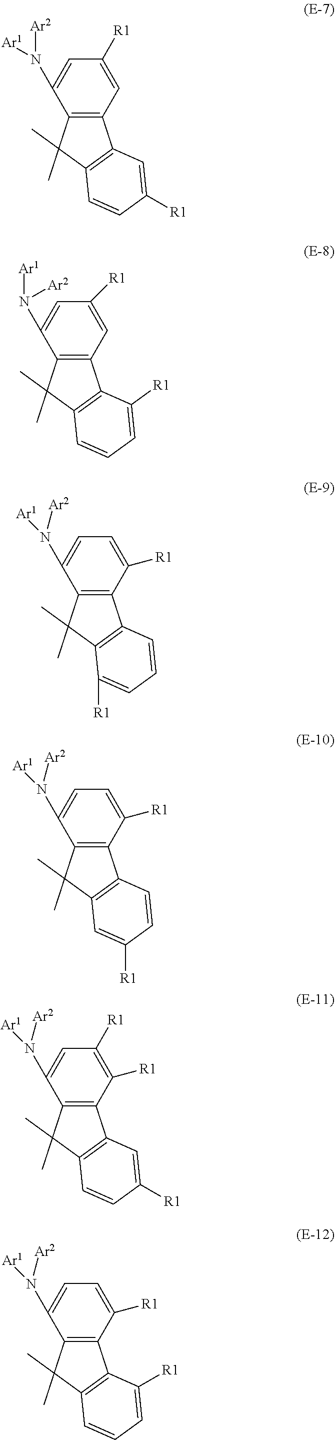

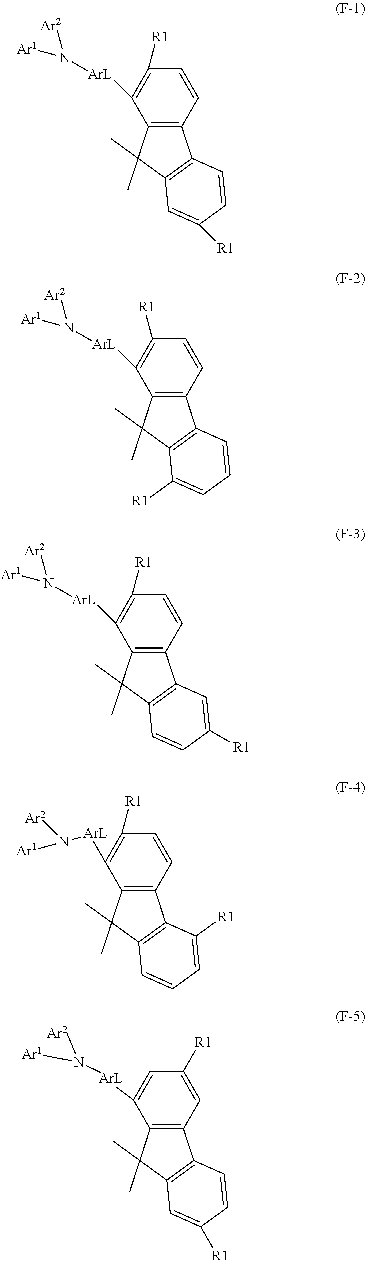

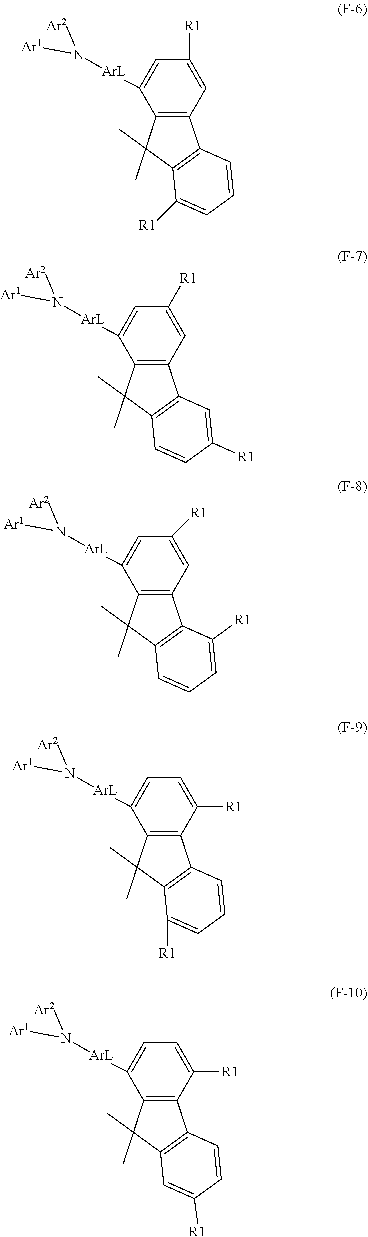

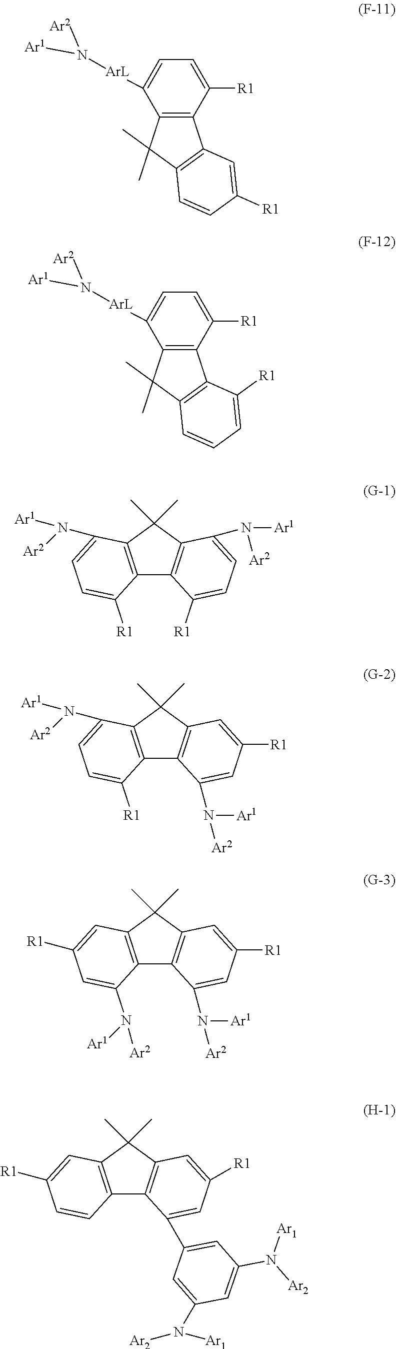

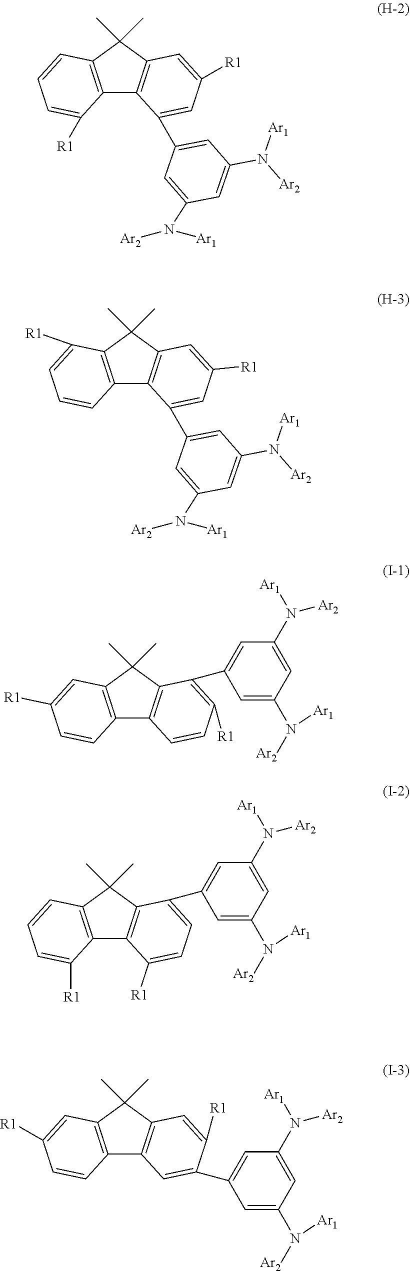

where the indices and groups that occur are as defined above, where “R1” corresponds to “R¹”, “ArL” corresponds to “Ar^{L}”, and where the groups and indices that occur preferably correspond to their preferred embodiments. Among the abovementioned formulae, preference is given to the formulae (A-1), (A-8), (A-10), (B-1), (B-8), (B-10), (C-1), (C-8), (C-11), (D-1), (D-3), (D-7), (D-11), (E-1), (E-4), (E-12), (F-1), (F-5), (F-8), (F-10), (F-12); especially preferred is formula (A-1).

Preferably, R² is the same or different at each instance and is selected from H, D, F, CN, Si(R⁷)₃ and —NAr¹Ar²; more preferably, R² is H.

Preferably, R³ is the same or different at each instance and is selected from H, D, F, CN, Si(R⁷)₃, N(R⁷)₂, —NAr¹Ar², straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C=0, C═NR⁷, —NR⁷—, —O—, —S—, —C(═O)O— or —C(═O)NR⁷—.

Preferably, R⁴ and R⁶ are the same or different at each instance and are selected from H, D, F, CN, Si(R⁷)₃, N(R⁷)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C═O, C═NR⁷, —NR⁷—, —O—, —S—, —C(═O)O— or —C(═O)NR⁷—.

Preferably, R⁵ is the same or different at each instance and is selected from straight-chain alkyl groups having 1 to 20 carbon atoms, and branched or cyclic alkyl groups having 3 to 20 carbon atoms, where said alkyl groups are each substituted by R⁷ radicals. Preferably, R⁷ in these cases is the same or different at each instance and is selected from H, D and F, and is most preferably H. More preferably, R⁵ is the same or different at each instance and is selected from methyl, ethyl, isopropyl, n-propyl, tert-butyl, isobutyl, n-butyl, cyclobutyl, cyclopentyl, cyclohexyl, CF₃, —CD₃, —CD₂CD₃, d7-n-propyl, d7-isopropyl, d9-n-butyl, d9-tert-butyl, d9-isobutyl, d7-cyclobutyl, d9-cyclopentyl and d11-cyclohexyl; most preferably, R⁵ is methyl.

Preferably, R⁵ is the same at each instance.

Preferably, R⁷ is the same or different at each instance and is selected from H, D, F, CN, Si(R⁸)₃, N(R⁸)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁸ radicals; and where one or more CH₂ groups in said alkyl groups may be replaced by —C≡C—, —R⁸C═CR⁸—, Si(R⁸)₂, C═O, C═NR⁸, —NR⁸—, —O—, —S—, —C(═O)O— or —C(═O)NR⁸—. More preferably, R⁷ is the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms. It is yet more preferable when R⁷ is H.

Especially preferred is a compound of formula (I) as shown above, where the variables that occur are as follows in combination:
Z¹ is C when an R¹ group or the group

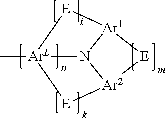

is bonded thereto, and is otherwise CR²;
the group

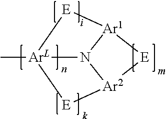

is bonded in the 4 position of the fluorenyl group of the formula (I);
Ar^{L} is phenylene substituted by R³ radicals, where R³ in this case is H;
Ar¹ is selected from aromatic ring systems which have 6 to 40 aromatic ring atoms and are substituted by R⁴ radicals, and heteroaromatic ring systems which have 5 to 40 aromatic ring atoms and are substituted by
R⁴ radicals;
Ar² is selected from aromatic ring systems which have 6 to 40 aromatic ring atoms and are substituted by R⁴ radicals, and heteroaromatic ring systems which have 5 to 40 aromatic ring atoms and are substituted by R⁴ radicals;
where at least one group selected from the Ar¹ and Ar² groups is selected from the following radicals: biphenyl; terphenyl; quaterphenyl; phenanthrenyl; dibenzofuranyl; dibenzothiophenyl; carbazolyl; and phenyl substituted by at least one group selected from fluorenyl, naphthyl, phenanthrenyl, dibenzofuranyl, dibenzothiophenyl, and carbazolyl, where these radicals are each substituted by R⁴ radicals;
R¹ is the same or different at each instance and is selected from straight-chain alkyl groups having 1 to 20 carbon atoms, branched or cyclic alkyl groups having 3 to 20 carbon atoms and aromatic ring systems having 6 to 40 aromatic ring atoms, where said alkyl groups and said aromatic ring systems are each substituted by R⁷ radicals, where R⁷ in this case is preferably H;
R² is H;
R³ is the same or different at each instance and is selected from H, D, F, CN, Si(R⁷)₃, N(R⁷)₂, —NAr¹Ar², straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C═O, C═NR⁷, —NR⁷—, —O—, —S—, —C(═O)O— or —C(═O)NR⁷—;
R⁴ and R⁶ are the same or different at each instance and are selected from H, D, F, CN, Si(R⁷)₃, N(R⁷)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C═O, C═NR⁷, —NR⁷—, —O—, —S—, —C(═O)O— or —C(═O)NR⁷—;
R⁵ is the same or different at each instance and is selected from straight-chain alkyl groups having 1 to 20 carbon atoms, and branched or cyclic alkyl groups having 3 to 20 carbon atoms, where said alkyl groups are each substituted by R⁷ radicals;
R⁷ is the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms;
n is 0 or 1, where, when n=0, Ar^{L} is absent, and the fluorene and the amino group in formula (I) are bonded directly to one another;
i, k and m are 0;
where the group

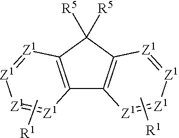

of the formula (I) in the compound is not identical to one or both of the Ar¹ and Ar² groups in the compound.

Preferred compounds of formula (I) are the following compounds:

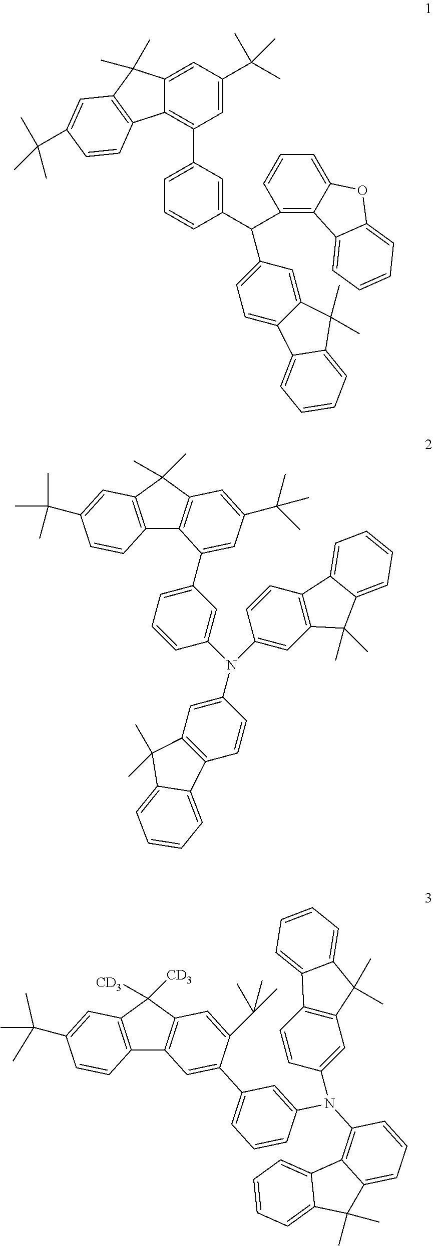 [SMILES: CC(C)(C)c1ccc2c(c1)C(C)(C)c1cc(C(C)(C)C)cc(-c3cccc(C(c4ccc5c(c4)C(C)(C)c4ccccc4-5)c4cccc5oc6ccccc6c45)c3)c1-2 ; CC(C)(C)c1ccc2c(c1)C(C)(C)c1cc(C(C)(C)C)cc(-c3cccc(N(c4ccc5c(c4)C(C)(C)c4ccccc4-5)c4ccc5c(c4)C(C)(C)c4ccccc4-5)c3)c1-2 ; [2H]C([2H])([2H])C1(C([2H])([2H])[2H])c2cc(C(C)(C)C)ccc2-c2cc(-c3cccc(N(c4ccc5c(c4)C(C)(C)c4ccccc4-5)c4cccc5c4-c4ccccc4C5(C)C)c3)c(C(C)(C)C)cc21]

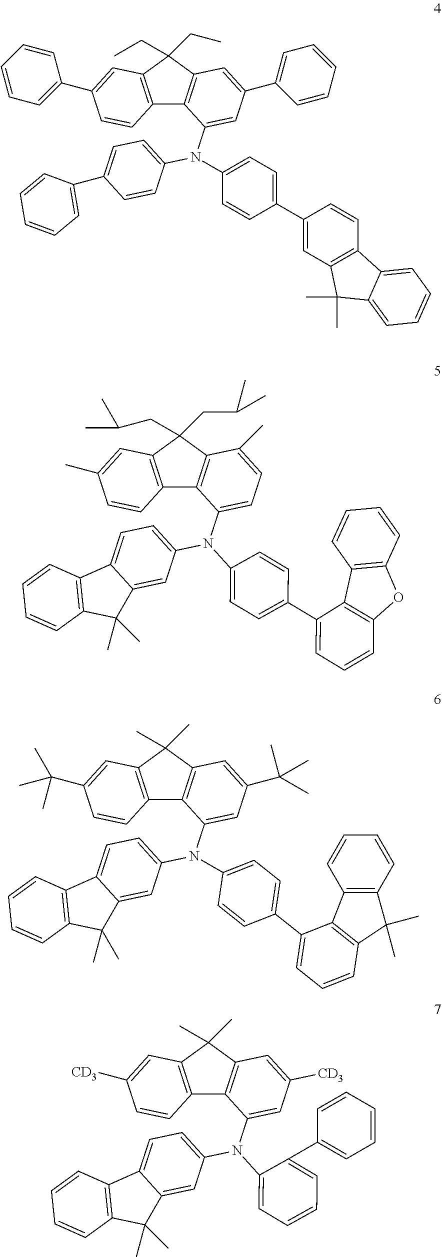 [SMILES: CCC1(CC)c2cc(-c3ccccc3)ccc2-c2c(N(c3ccc(-c4ccccc4)cc3)c3ccc(-c4ccc5c(c4)C(C)(C)c4ccccc4-5)cc3)cc(-c3ccccc3)cc21 ; Cc1ccc2c(c1)C(CC(C)C)(CC(C)C)c1c(C)ccc(N(c3ccc(-c4cccc5oc6ccccc6c45)cc3)c3ccc4c(c3)C(C)(C)c3ccccc3-4)c1-2 ; CC(C)(C)c1ccc2c(c1)C(C)(C)c1cc(C(C)(C)C)cc(N(c3ccc(-c4cccc5c4-c4ccccc4C5(C)C)cc3)c3ccc4c(c3)C(C)(C)c3ccccc3-4)c1-2 ; [2H]C([2H])([2H])c1ccc2c(c1)C(C)(C)c1cc(C([2H])([2H])[2H])cc(N(c3ccc4c(c3)C(C)(C)c3ccccc3-4)c3ccccc3-c3ccccc3)c1-2]

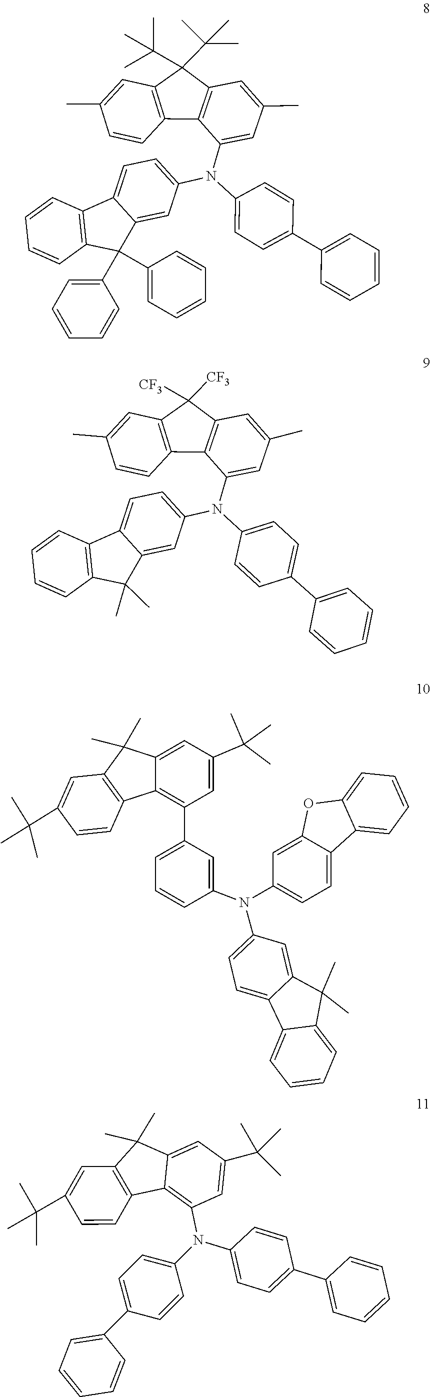

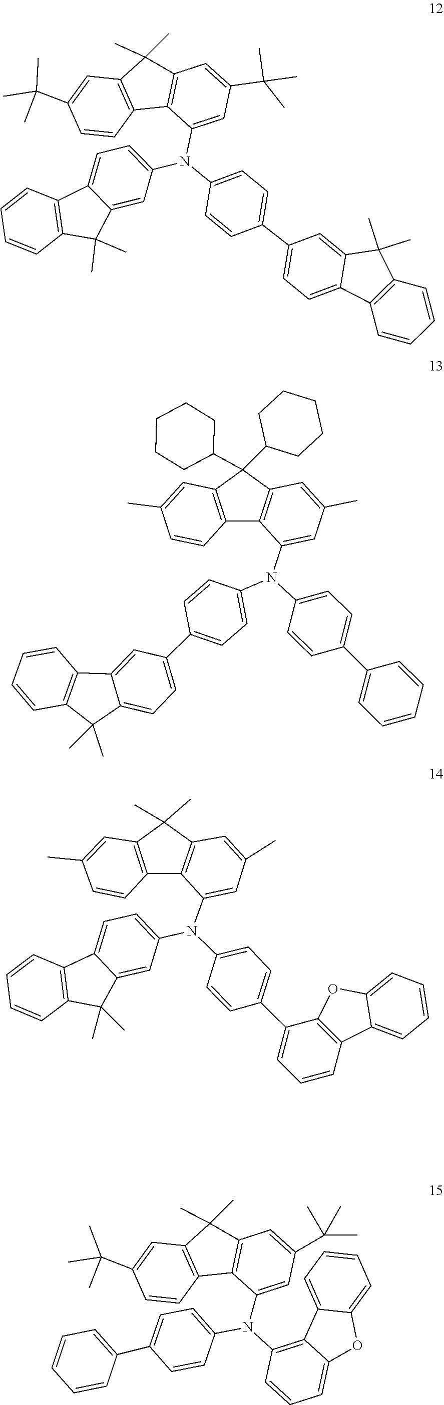 [SMILES: CC(C)(C)c1ccc2c(c1)C(C)(C)c1cc(C(C)(C)C)cc(N(c3ccc(-c4ccc5c(c4)C(C)(C)c4ccccc4-5)cc3)c3ccc4c(c3)C(C)(C)c3ccccc3-4)c1-2 ; Cc1ccc2c(c1)C(C1CCCCC1)(C1CCCCC1)c1cc(C)cc(N(c3ccc(-c4ccccc4)cc3)c3ccc(-c4ccc5c(c4)-c4ccccc4C5(C)C)cc3)c1-2 ; Cc1ccc2c(c1)C(C)(C)c1cc(C)cc(N(c3ccc(-c4cccc5c4oc4ccccc45)cc3)c3ccc4c(c3)C(C)(C)c3ccccc3-4)c1-2 ; CC(C)(C)c1ccc2c(c1)C(C)(C)c1cc(C(C)(C)C)cc(N(c3ccc(-c4ccccc4)cc3)c3cccc4oc5ccccc5c34)c1-2]

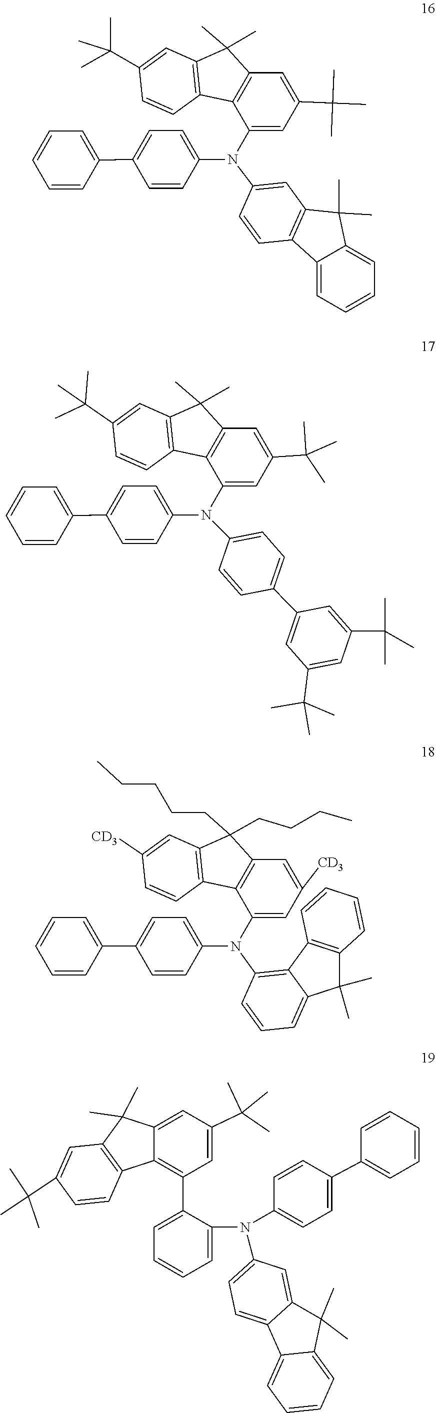

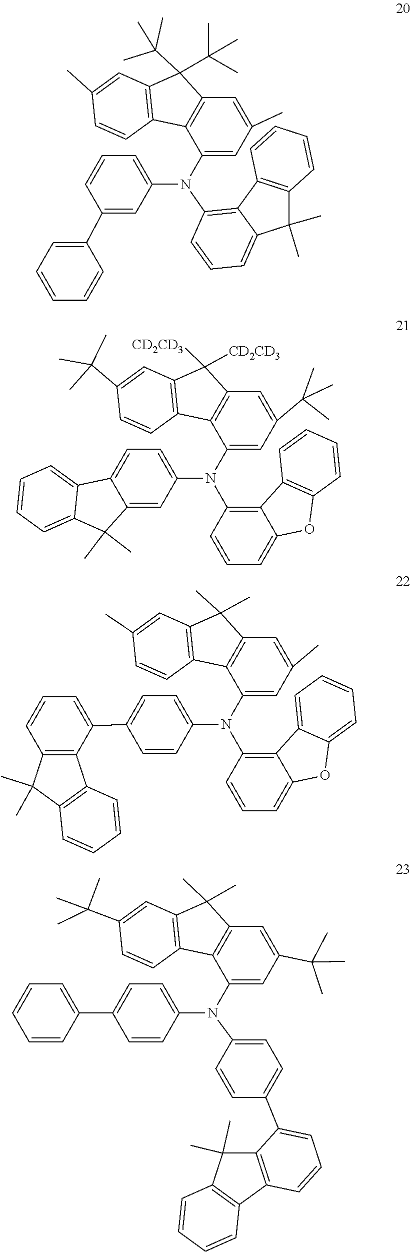

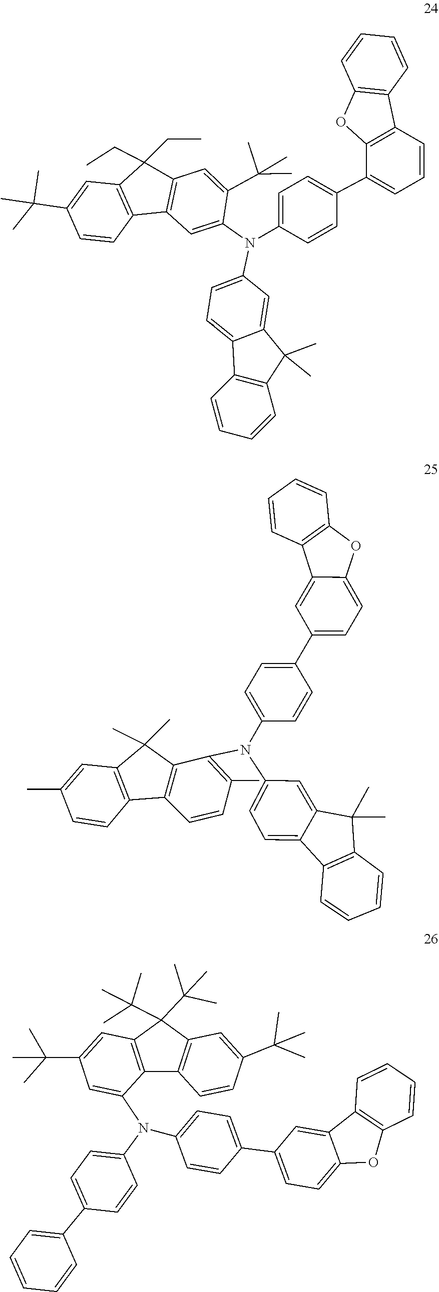

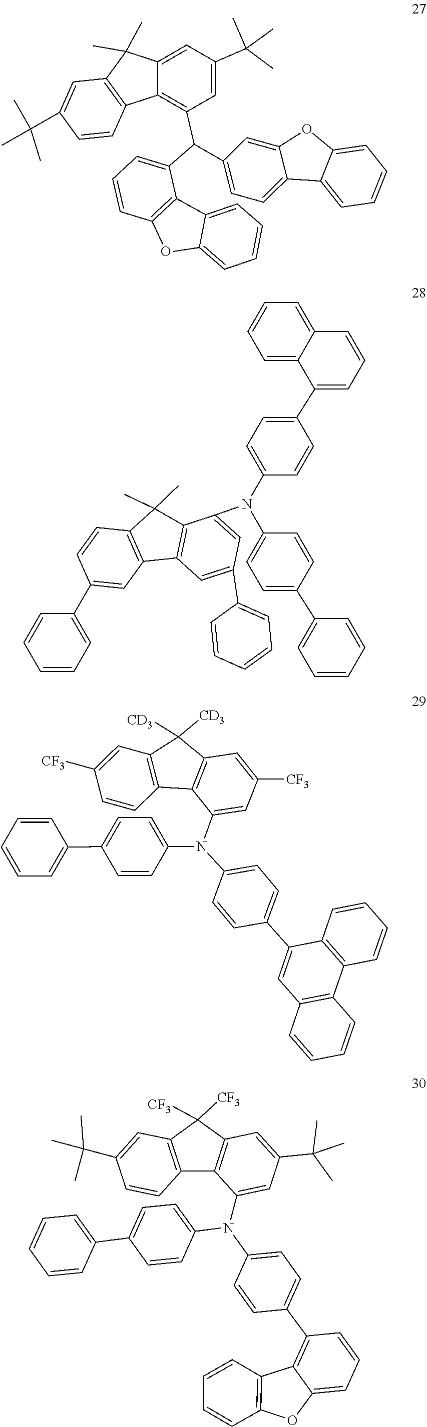

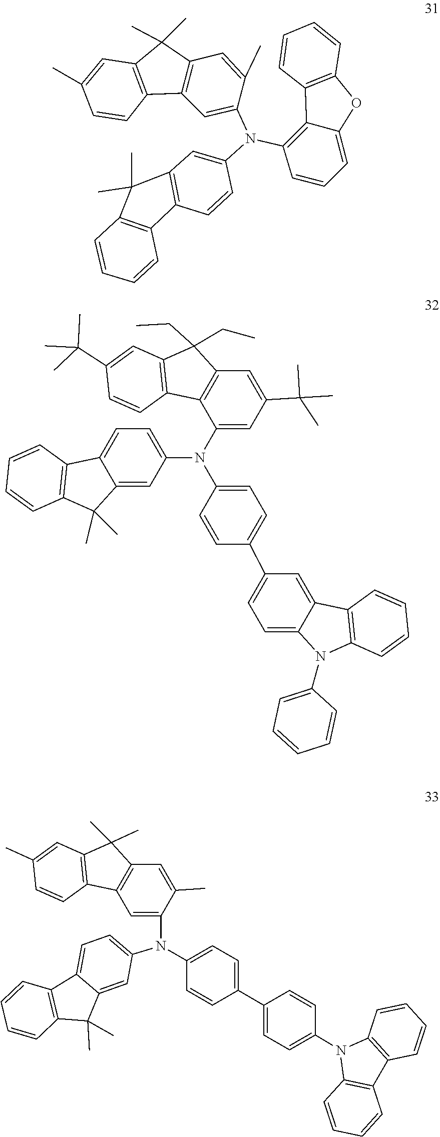

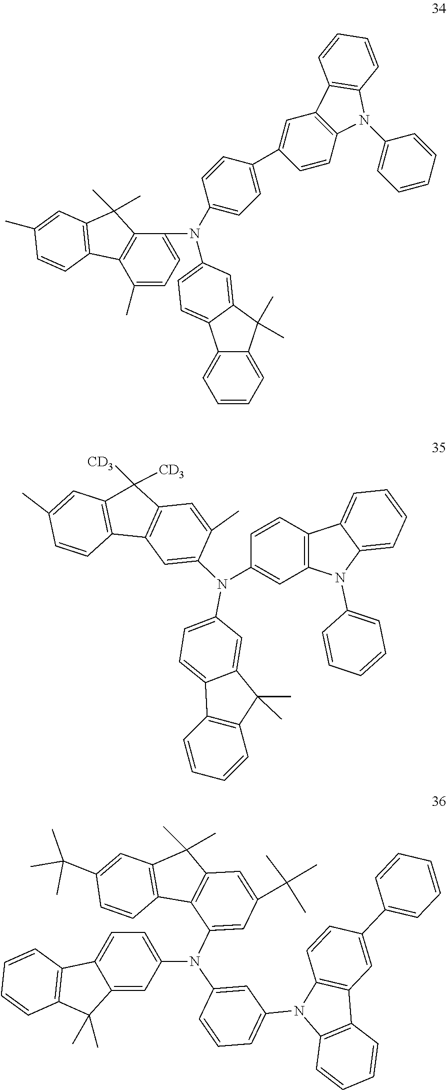 [SMILES: Cc1ccc2c(c1)C(C)(C)c1c(N(c3ccc(-c4ccc5c(c4)c4ccccc4n5-c4ccccc4)cc3)c3ccc4c(c3)C(C)(C)c3ccccc3-4)ccc(C)c1-2 ; [2H]C([2H])([2H])C1(C([2H])([2H])[2H])c2cc(C)ccc2-c2cc(N(c3ccc4c(c3)C(C)(C)c3ccccc3-4)c3ccc4c5ccccc5n(-c5ccccc5)c4c3)c(C)cc21 ; CC(C)(C)c1ccc2c(c1)C(C)(C)c1cc(C(C)(C)C)cc(N(c3cccc(-n4c5ccccc5c5cc(-c6ccccc6)ccc54)c3)c3ccc4c(c3)C(C)(C)c3ccccc3-4)c1-2]

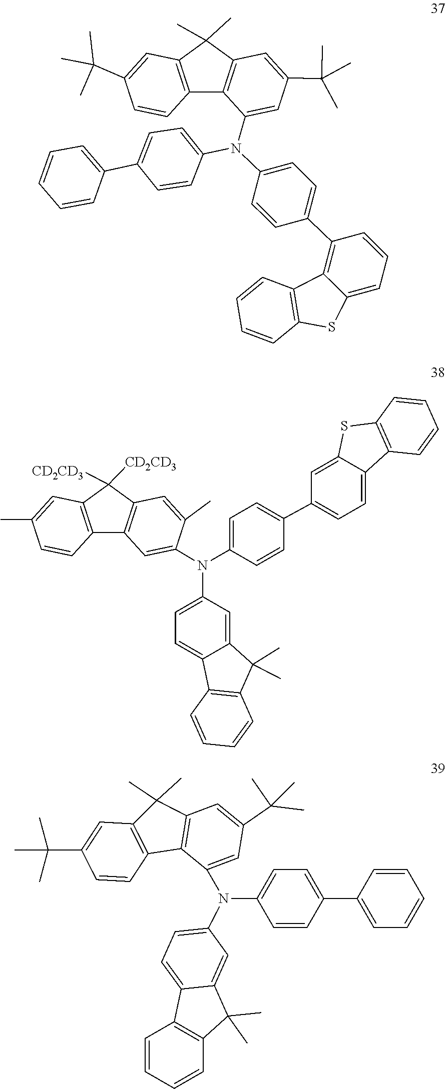

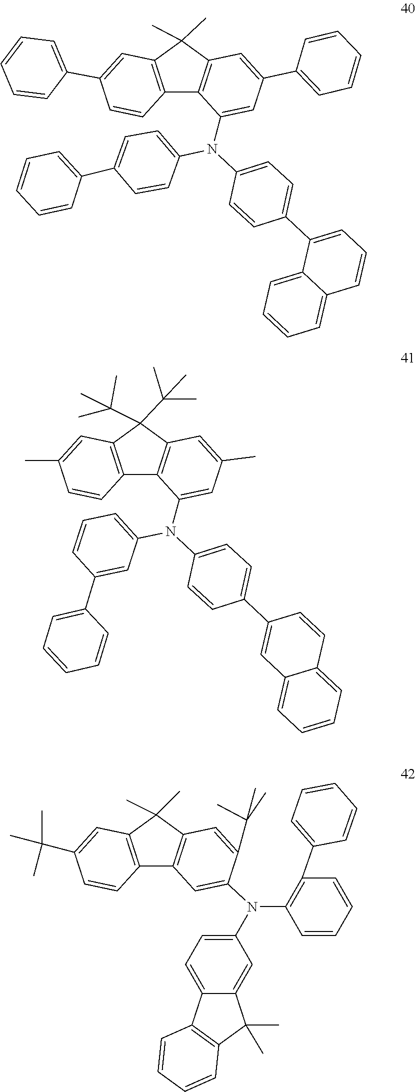 [SMILES: CC1(C)c2cc(-c3ccccc3)ccc2-c2c(N(c3ccc(-c4ccccc4)cc3)c3ccc(-c4cccc5ccccc45)cc3)cc(-c3ccccc3)cc21 ; Cc1ccc2c(c1)C(C(C)(C)C)(C(C)(C)C)c1cc(C)cc(N(c3ccc(-c4ccc5ccccc5c4)cc3)c3cccc(-c4ccccc4)c3)c1-2 ; CC(C)(C)c1ccc2c(c1)C(C)(C)c1cc(C(C)(C)C)c(N(c3ccc4c(c3)C(C)(C)c3ccccc3-4)c3ccccc3-c3ccccc3)cc1-2]

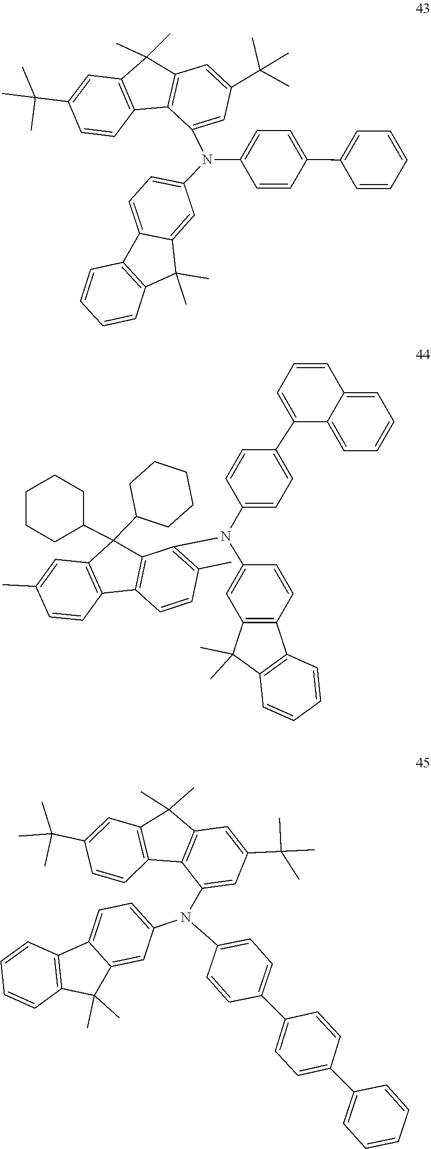 [SMILES: CC(C)(C)c1ccc2c(c1)C(C)(C)c1cc(C(C)(C)C)cc(N(c3ccc(-c4ccccc4)cc3)c3ccc4c(c3)C(C)(C)c3ccccc3-4)c1-2 ; Cc1ccc2c(c1)C(C1CCCCC1)(C1CCCCC1)c1c-2ccc(C)c1N(c1ccc(-c2cccc3ccccc23)cc1)c1ccc2c(c1)C(C)(C)c1ccccc1-2 ; CC(C)(C)c1ccc2c(c1)C(C)(C)c1cc(C(C)(C)C)cc(N(c3ccc(-c4ccc(-c5ccccc5)cc4)cc3)c3ccc4c(c3)C(C)(C)c3ccccc3-4)c1-2]

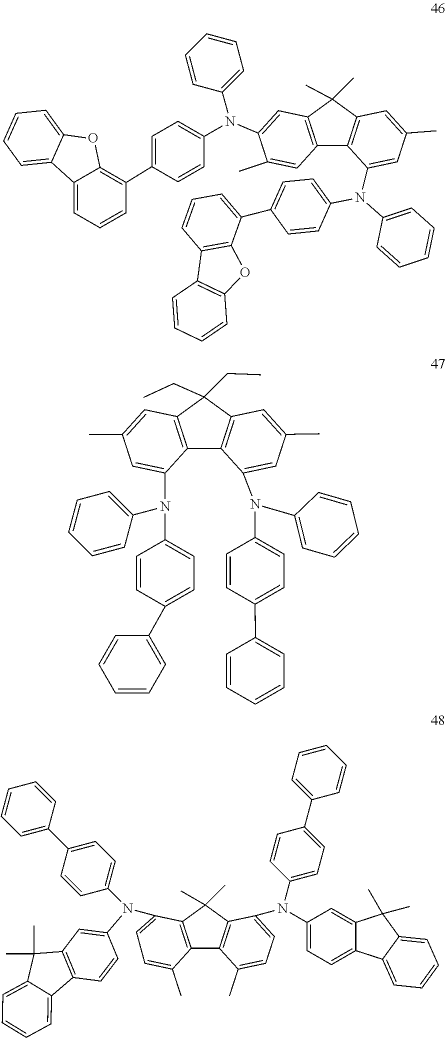

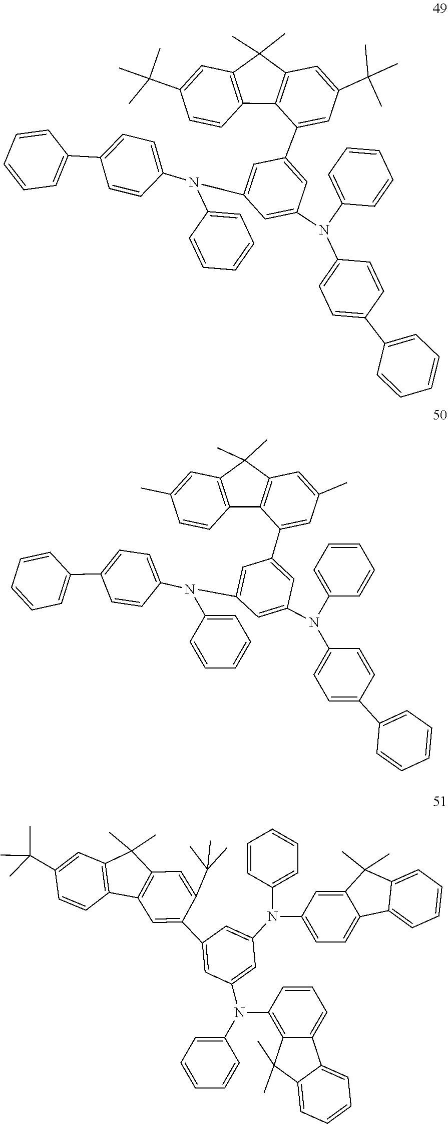 [SMILES: CC(C)(C)c1ccc2c(c1)C(C)(C)c1cc(C(C)(C)C)cc(-c3cc(N(c4ccccc4)c4ccc(-c5ccccc5)cc4)cc(N(c4ccccc4)c4ccc(-c5ccccc5)cc4)c3)c1-2 ; Cc1ccc2c(c1)C(C)(C)c1cc(C)cc(-c3cc(N(c4ccccc4)c4ccc(-c5ccccc5)cc4)cc(N(c4ccccc4)c4ccc(-c5ccccc5)cc4)c3)c1-2 ; CC(C)(C)c1ccc2c(c1)C(C)(C)c1cc(C(C)(C)C)c(-c3cc(N(c4ccccc4)c4ccc5c(c4)C(C)(C)c4ccccc4-5)cc(N(c4ccccc4)c4cccc5c4C(C)(C)c4ccccc4-5)c3)cc1-2]

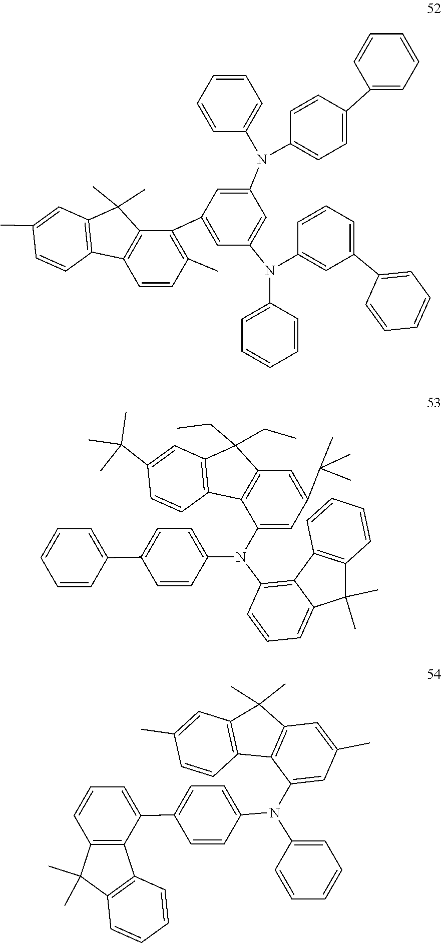

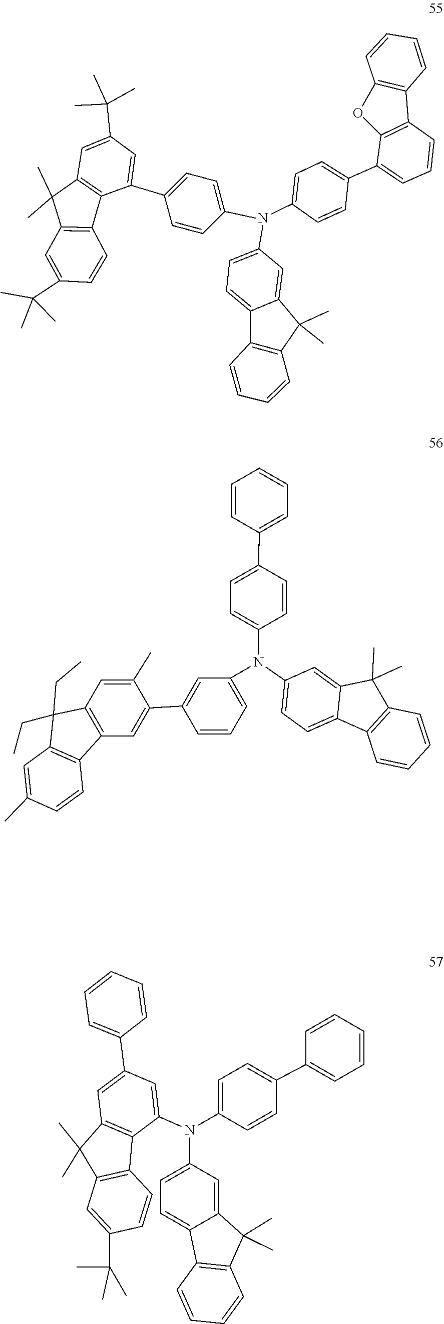 [SMILES: CC(C)(C)c1ccc2c(c1)C(C)(C)c1cc(C(C)(C)C)cc(-c3ccc(N(c4ccc(-c5cccc6c5oc5ccccc56)cc4)c4ccc5c(c4)C(C)(C)c4ccccc4-5)cc3)c1-2 ; CCC1(CC)c2cc(C)ccc2-c2cc(-c3cccc(N(c4ccc(-c5ccccc5)cc4)c4ccc5c(c4)C(C)(C)c4ccccc4-5)c3)c(C)cc21 ; CC(C)(C)c1ccc2c(c1)C(C)(C)c1cc(-c3ccccc3)cc(N(c3ccc(-c4ccccc4)cc3)c3ccc4c(c3)C(C)(C)c3ccccc3-4)c1-2]

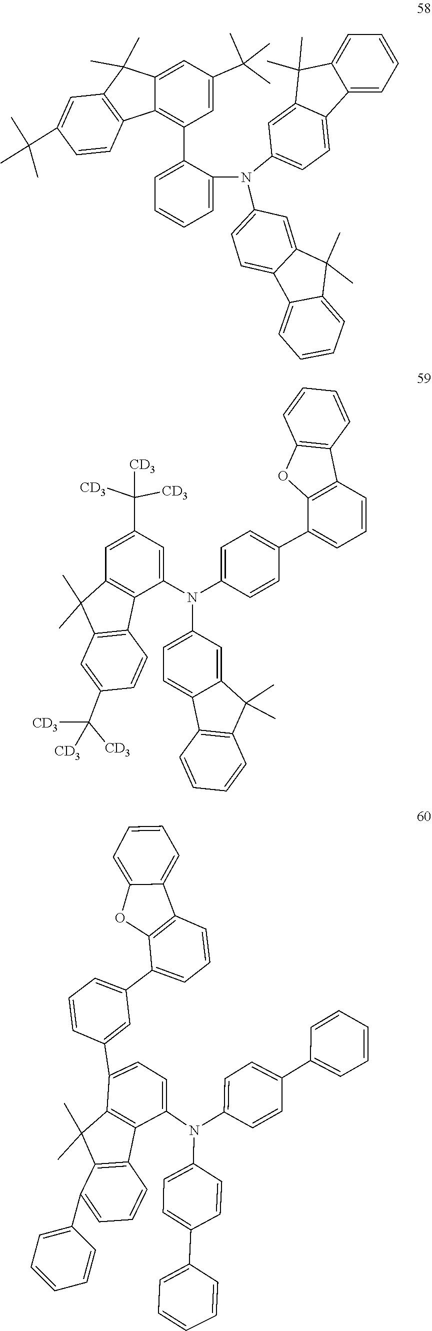

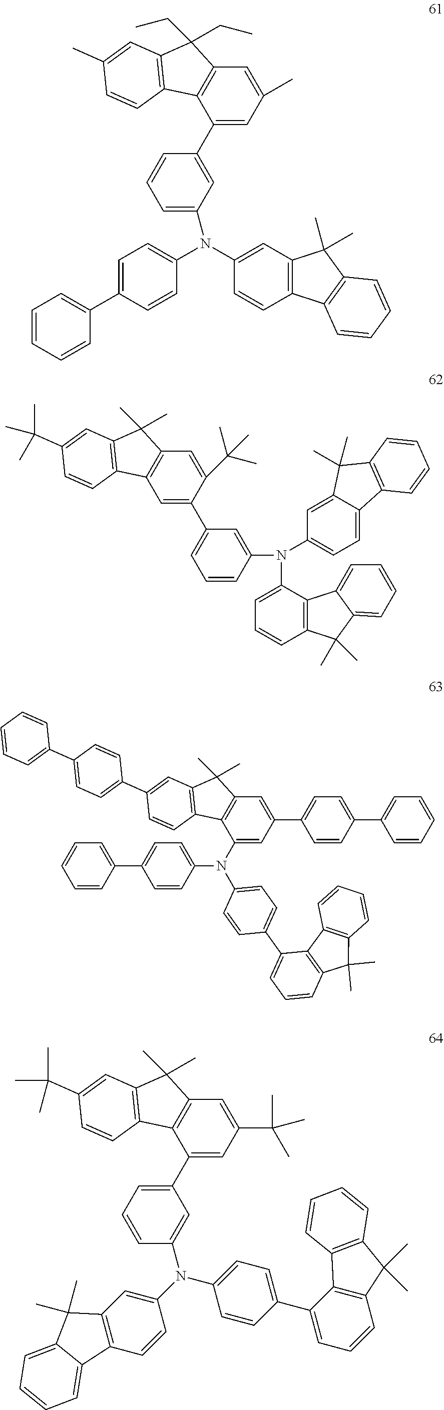 [SMILES: CCC1(CC)c2cc(C)ccc2-c2c(-c3cccc(N(c4ccc(-c5ccccc5)cc4)c4ccc5c(c4)C(C)(C)c4ccccc4-5)c3)cc(C)cc21 ; CC(C)(C)c1ccc2c(c1)C(C)(C)c1cc(C(C)(C)C)c(-c3cccc(N(c4ccc5c(c4)C(C)(C)c4ccccc4-5)c4cccc5c4-c4ccccc4C5(C)C)c3)cc1-2 ; CC1(C)c2ccccc2-c2c(-c3ccc(N(c4ccc(-c5ccccc5)cc4)c4cc(-c5ccc(-c6ccccc6)cc5)cc5c4-c4ccc(-c6ccc(-c7ccccc7)cc6)cc4C5(C)C)cc3)cccc21 ; CC(C)(C)c1ccc2c(c1)C(C)(C)c1cc(C(C)(C)C)cc(-c3cccc(N(c4ccc(-c5cccc6c5-c5ccccc5C6(C)C)cc4)c4ccc5c(c4)C(C)(C)c4ccccc4-5)c3)c1-2]

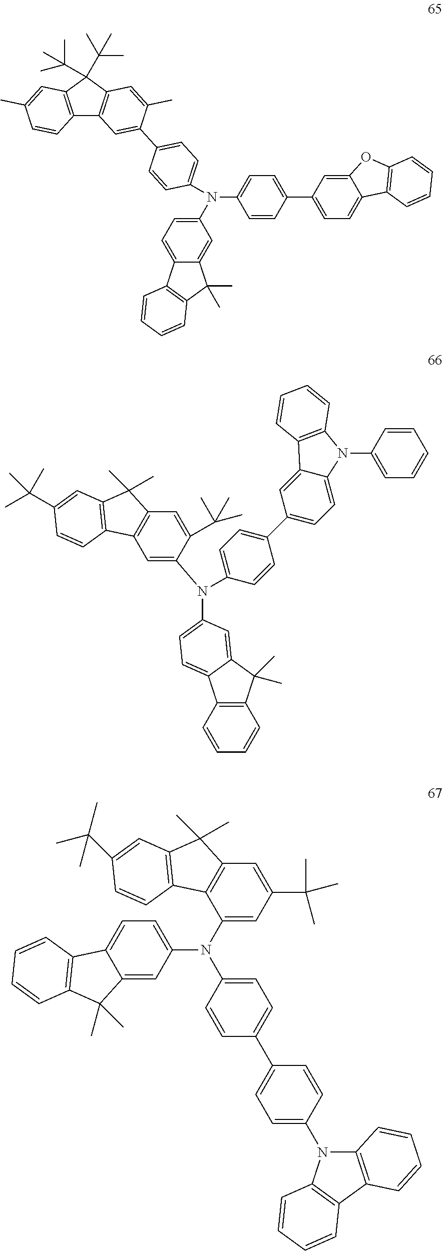 [SMILES: Cc1ccc2c(c1)C(C(C)(C)C)(C(C)(C)C)c1cc(C)c(-c3ccc(N(c4ccc(-c5ccc6c(c5)oc5ccccc56)cc4)c4ccc5c(c4)C(C)(C)c4ccccc4-5)cc3)cc1-2 ; CC(C)(C)c1ccc2c(c1)C(C)(C)c1cc(C(C)(C)C)c(N(c3ccc(-c4ccc5c(c4)c4ccccc4n5-c4ccccc4)cc3)c3ccc4c(c3)C(C)(C)c3ccccc3-4)cc1-2 ; CC(C)(C)c1ccc2c(c1)C(C)(C)c1cc(C(C)(C)C)cc(N(c3ccc(-c4ccc(-n5c6ccccc6c6ccccc65)cc4)cc3)c3ccc4c(c3)C(C)(C)c3ccccc3-4)c1-2]

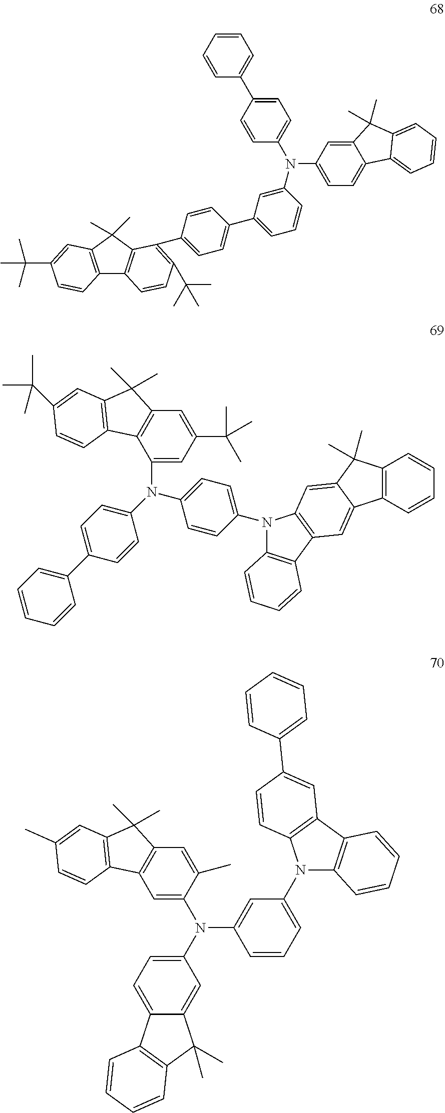 [SMILES: CC(C)(C)c1ccc2c(c1)C(C)(C)c1c-2ccc(C(C)(C)C)c1-c1ccc(-c2cccc(N(c3ccc(-c4ccccc4)cc3)c3ccc4c(c3)C(C)(C)c3ccccc3-4)c2)cc1 ; CC(C)(C)c1ccc2c(c1)C(C)(C)c1cc(C(C)(C)C)cc(N(c3ccc(-c4ccccc4)cc3)c3ccc(-n4c5ccccc5c5cc6c(cc54)C(C)(C)c4ccccc4-6)cc3)c1-2 ; Cc1ccc2c(c1)C(C)(C)c1cc(C)c(N(c3cccc(-n4c5ccccc5c5cc(-c6ccccc6)ccc54)c3)c3ccc4c(c3)C(C)(C)c3ccccc3-4)cc1-2]

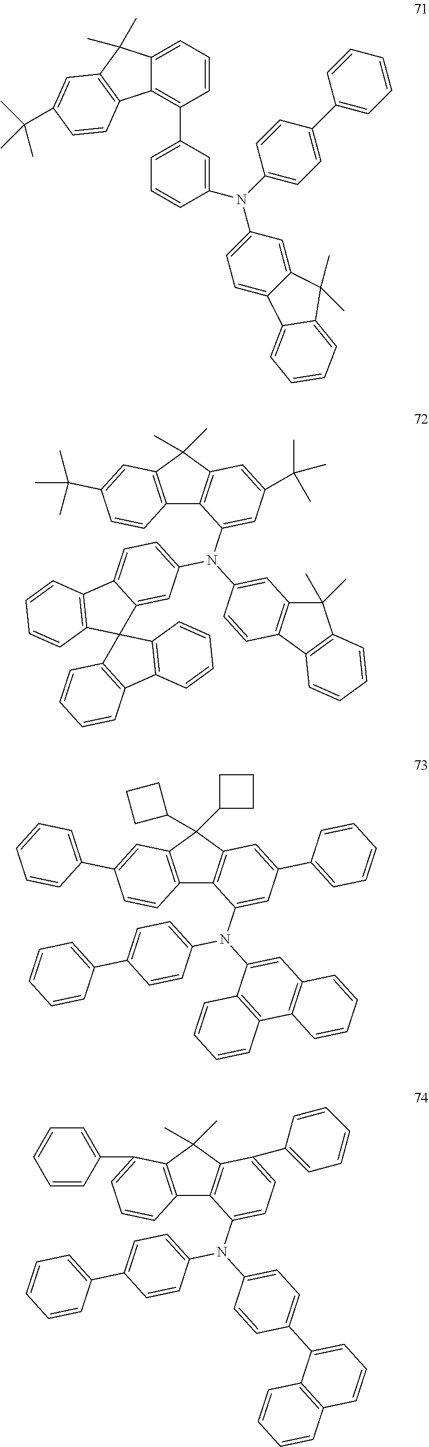 [SMILES: CC(C)(C)c1ccc2c(c1)C(C)(C)c1cccc(-c3cccc(N(c4ccc(-c5ccccc5)cc4)c4ccc5c(c4)C(C)(C)c4ccccc4-5)c3)c1-2 ; CC(C)(C)c1ccc2c(c1)C(C)(C)c1cc(C(C)(C)C)cc(N(c3ccc4c(c3)C(C)(C)c3ccccc3-4)c3ccc4c(c3)C3(c5ccccc5-c5ccccc53)c3ccccc3-4)c1-2 ; c1ccc(-c2ccc(N(c3cc(-c4ccccc4)cc4c3-c3ccc(-c5ccccc5)cc3C4(C3CCC3)C3CCC3)c3cc4ccccc4c4ccccc34)cc2)cc1 ; CC1(C)c2c(-c3ccccc3)cccc2-c2c(N(c3ccc(-c4ccccc4)cc3)c3ccc(-c4cccc5ccccc45)cc3)ccc(-c3ccccc3)c21]

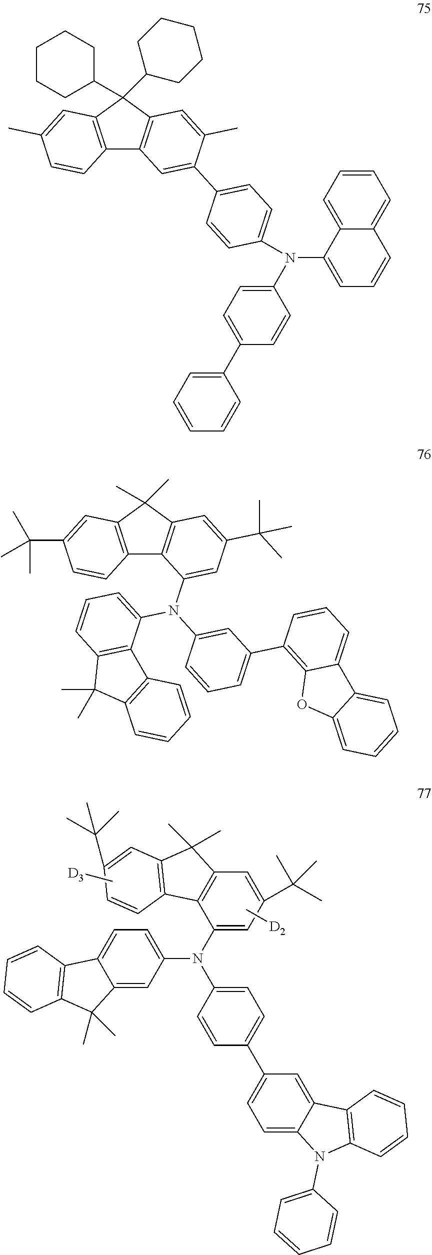

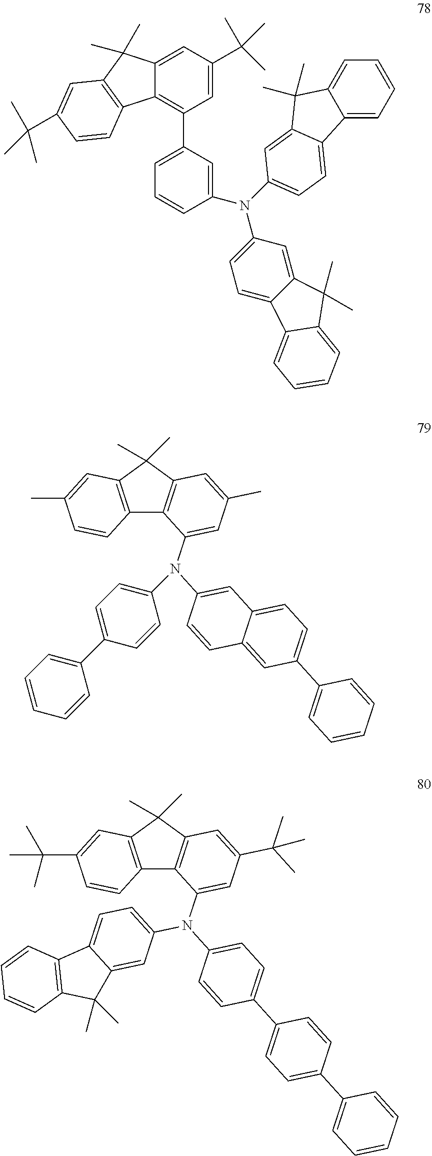 [SMILES: CC(C)(C)c1ccc2c(c1)C(C)(C)c1cc(C(C)(C)C)cc(-c3cccc(N(c4ccc5c(c4)C(C)(C)c4ccccc4-5)c4ccc5c(c4)C(C)(C)c4ccccc4-5)c3)c1-2 ; Cc1ccc2c(c1)C(C)(C)c1cc(C)cc(N(c3ccc(-c4ccccc4)cc3)c3ccc4cc(-c5ccccc5)ccc4c3)c1-2 ; CC(C)(C)c1ccc2c(c1)C(C)(C)c1cc(C(C)(C)C)cc(N(c3ccc(-c4ccc(-c5ccccc5)cc4)cc3)c3ccc4c(c3)C(C)(C)c3ccccc3-4)c1-2]

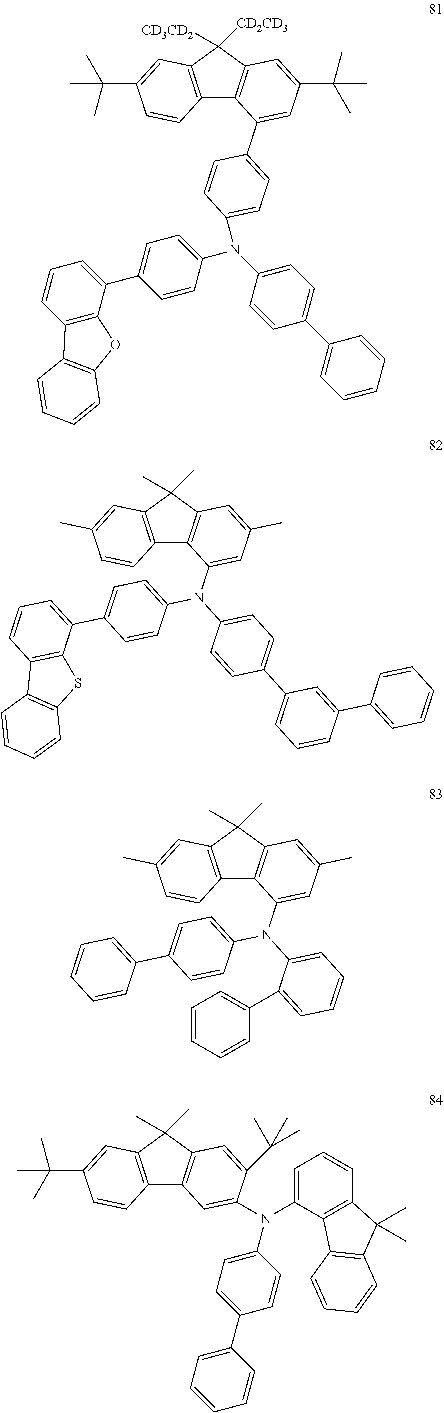

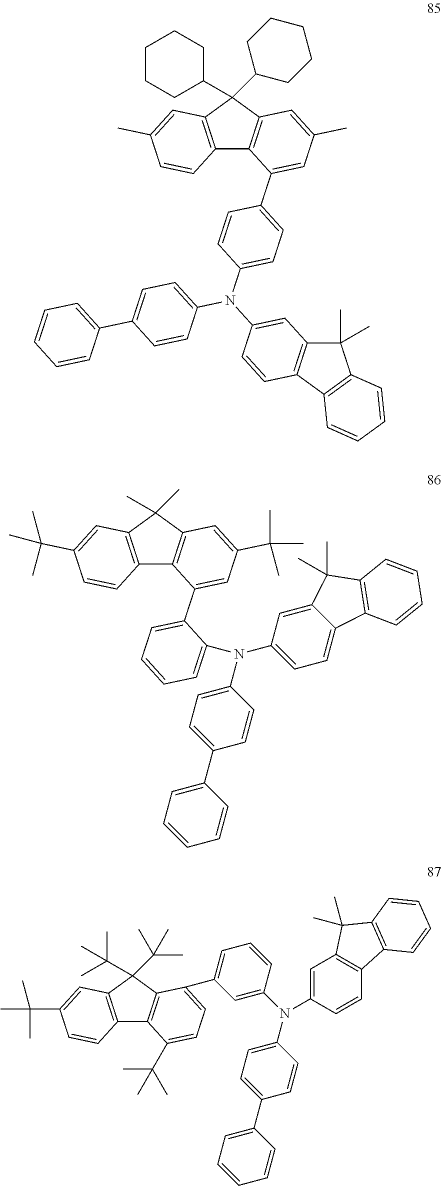 [SMILES: Cc1ccc2c(c1)C(C1CCCCC1)(C1CCCCC1)c1cc(C)cc(-c3ccc(N(c4ccc(-c5ccccc5)cc4)c4ccc5c(c4)C(C)(C)c4ccccc4-5)cc3)c1-2 ; CC(C)(C)c1ccc2c(c1)C(C)(C)c1cc(C(C)(C)C)cc(-c3ccccc3N(c3ccc(-c4ccccc4)cc3)c3ccc4c(c3)C(C)(C)c3ccccc3-4)c1-2 ; CC(C)(C)c1ccc2c(c1)C(C(C)(C)C)(C(C)(C)C)c1c(-c3cccc(N(c4ccc(-c5ccccc5)cc4)c4ccc5c(c4)C(C)(C)c4ccccc4-5)c3)ccc(C(C)(C)C)c1-2]

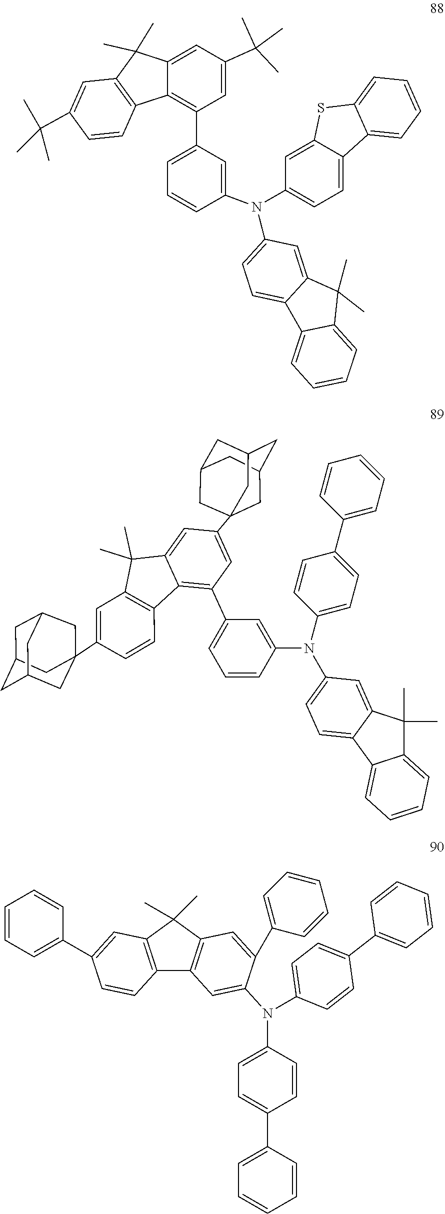 [SMILES: CC(C)(C)c1ccc2c(c1)C(C)(C)c1cc(C(C)(C)C)cc(-c3cccc(N(c4ccc5c(c4)C(C)(C)c4ccccc4-5)c4ccc5c(c4)sc4ccccc45)c3)c1-2 ; CC1(C)c2ccccc2-c2ccc(N(c3ccc(-c4ccccc4)cc3)c3cccc(-c4cc(C56CC7CC(CC(C7)C5)C6)cc5c4-c4ccc(C67CC8CC(CC(C8)C6)C7)cc4C5(C)C)c3)cc21 ; CC1(C)c2cc(-c3ccccc3)ccc2-c2cc(N(c3ccc(-c4ccccc4)cc3)c3ccc(-c4ccccc4)cc3)c(-c3ccccc3)cc21]

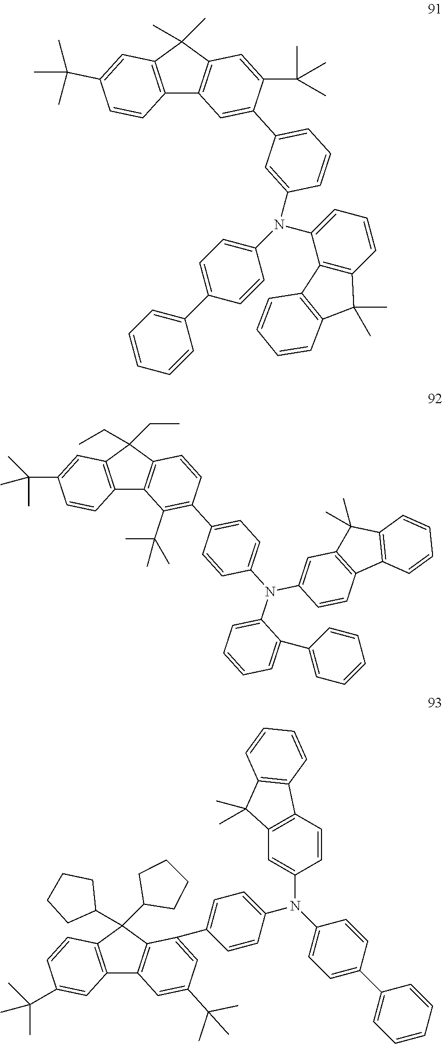

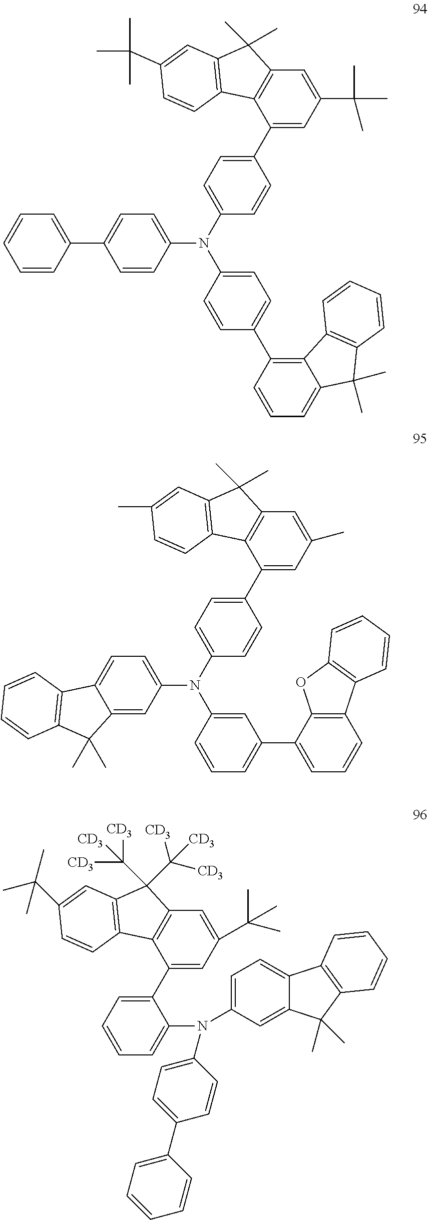

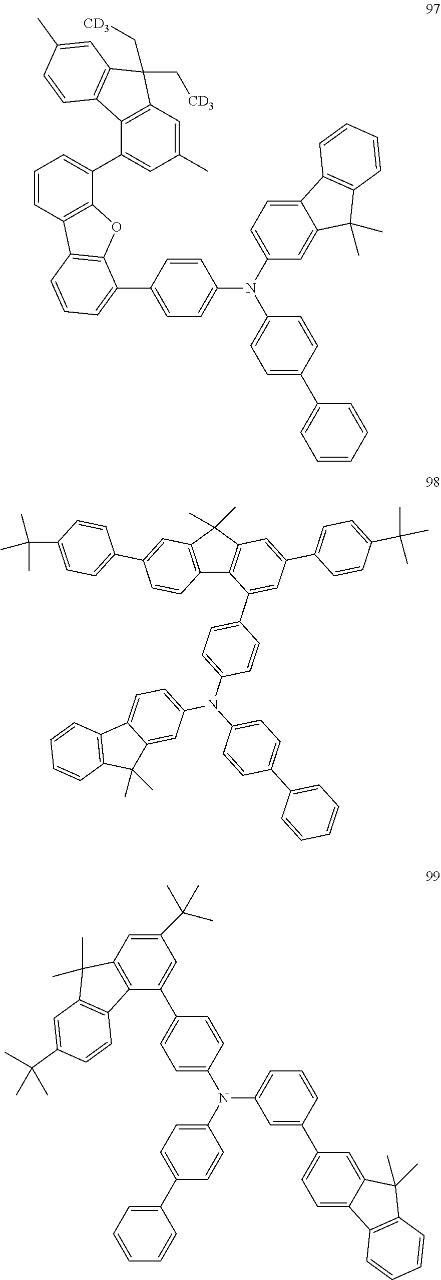

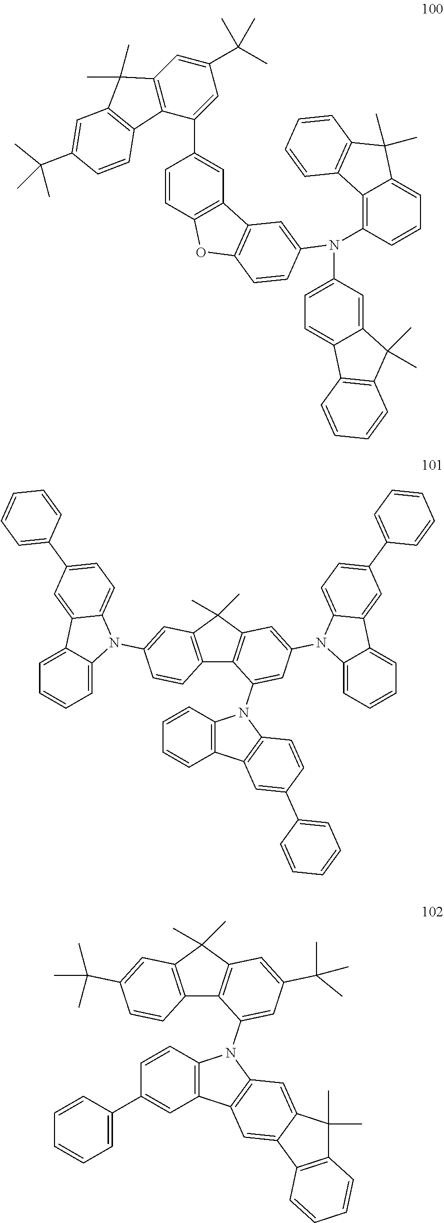 [SMILES: CC(C)(C)c1ccc2c(c1)C(C)(C)c1cc(C(C)(C)C)cc(-c3ccc4oc5ccc(N(c6ccc7c(c6)C(C)(C)c6ccccc6-7)c6cccc7c6-c6ccccc6C7(C)C)cc5c4c3)c1-2 ; CC1(C)c2cc(-n3c4ccccc4c4cc(-c5ccccc5)ccc43)ccc2-c2c(-n3c4ccccc4c4cc(-c5ccccc5)ccc43)cc(-n3c4ccccc4c4cc(-c5ccccc5)ccc43)cc21 ; CC(C)(C)c1ccc2c(c1)C(C)(C)c1cc(C(C)(C)C)cc(-n3c4ccc(-c5ccccc5)cc4c4cc5c(cc43)C(C)(C)c3ccccc3-5)c1-2]

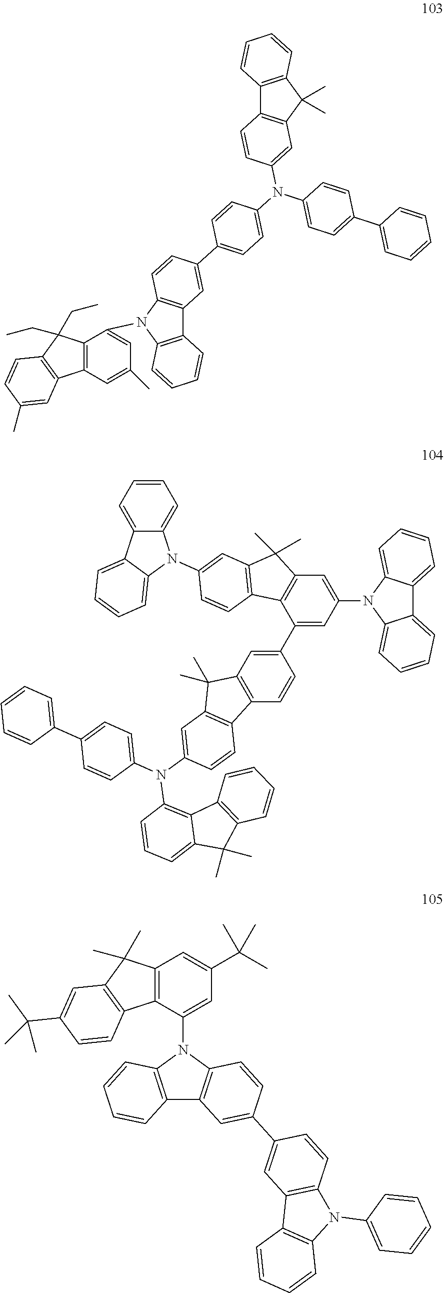 [SMILES: CCC1(CC)c2ccc(C)cc2-c2cc(C)cc(-n3c4ccccc4c4cc(-c5ccc(N(c6ccc(-c7ccccc7)cc6)c6ccc7c(c6)C(C)(C)c6ccccc6-7)cc5)ccc43)c21 ; CC1(C)c2cc(-c3cc(-n4c5ccccc5c5ccccc54)cc4c3-c3ccc(-n5c6ccccc6c6ccccc65)cc3C4(C)C)ccc2-c2ccc(N(c3ccc(-c4ccccc4)cc3)c3cccc4c3-c3ccccc3C4(C)C)cc21 ; CC(C)(C)c1ccc2c(c1)C(C)(C)c1cc(C(C)(C)C)cc(-n3c4ccccc4c4cc(-c5ccc6c(c5)c5ccccc5n6-c5ccccc5)ccc43)c1-2]

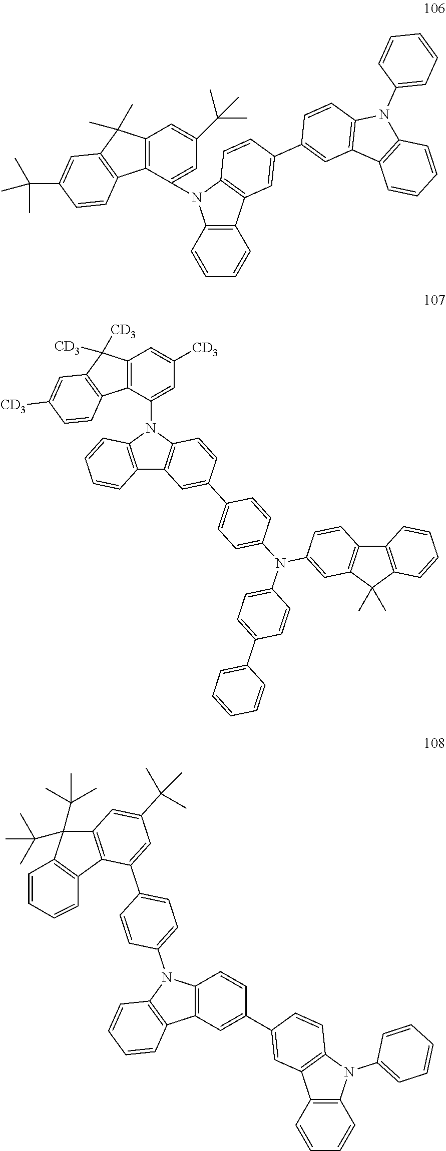 [SMILES: CC(C)(C)c1ccc2c(c1)C(C)(C)c1cc(C(C)(C)C)cc(-n3c4ccccc4c4cc(-c5ccc6c(c5)c5ccccc5n6-c5ccccc5)ccc43)c1-2 ; [2H]C([2H])([2H])c1ccc2c(c1)C(C([2H])([2H])[2H])(C([2H])([2H])[2H])c1cc(C([2H])([2H])[2H])cc(-n3c4ccccc4c4cc(-c5ccc(N(c6ccc(-c7ccccc7)cc6)c6ccc7c(c6)C(C)(C)c6ccccc6-7)cc5)ccc43)c1-2 ; CC(C)(C)c1cc(-c2ccc(-n3c4ccccc4c4cc(-c5ccc6c(c5)c5ccccc5n6-c5ccccc5)ccc43)cc2)c2c(c1)C(C(C)(C)C)(C(C)(C)C)c1ccccc1-2]

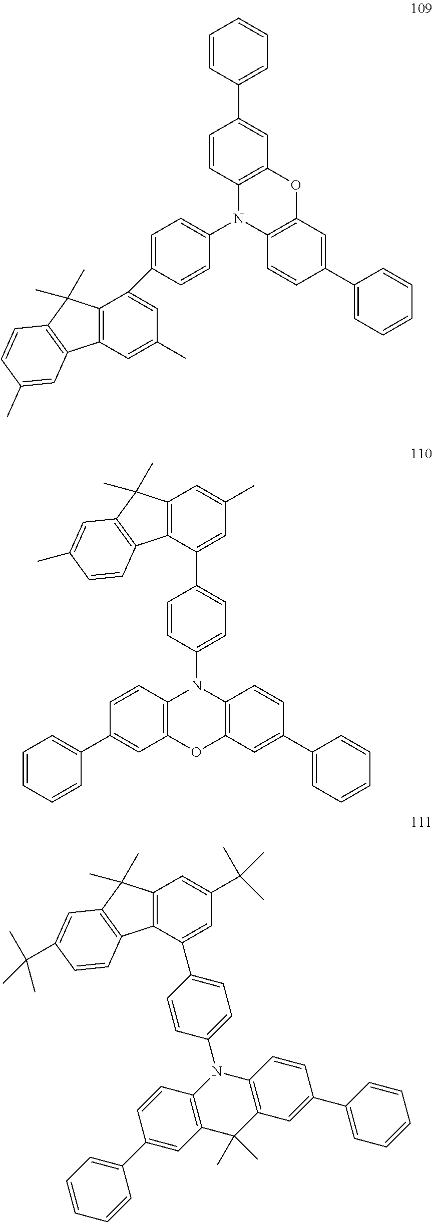 [SMILES: Cc1ccc2c(c1)-c1cc(C)cc(-c3ccc(N4c5ccc(-c6ccccc6)cc5Oc5cc(-c6ccccc6)ccc54)cc3)c1C2(C)C ; Cc1ccc2c(c1)C(C)(C)c1cc(C)cc(-c3ccc(N4c5ccc(-c6ccccc6)cc5Oc5cc(-c6ccccc6)ccc54)cc3)c1-2 ; CC(C)(C)c1ccc2c(c1)C(C)(C)c1cc(C(C)(C)C)cc(-c3ccc(N4c5ccc(-c6ccccc6)cc5C(C)(C)c5cc(-c6ccccc6)ccc54)cc3)c1-2]

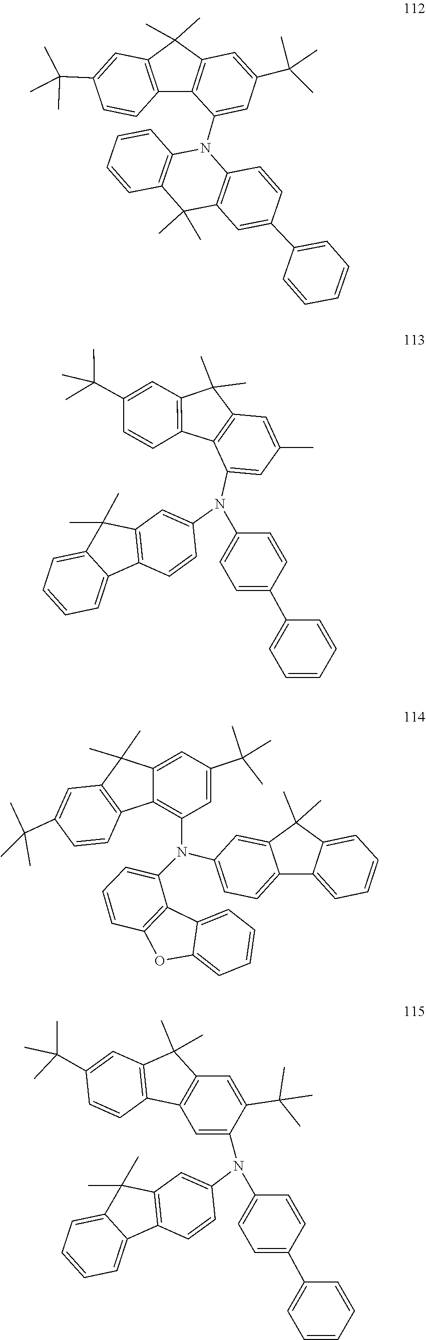 [SMILES: CC(C)(C)c1ccc2c(c1)C(C)(C)c1cc(C(C)(C)C)cc(N3c4ccccc4C(C)(C)c4cc(-c5ccccc5)ccc43)c1-2 ; Cc1cc(N(c2ccc(-c3ccccc3)cc2)c2ccc3c(c2)C(C)(C)c2ccccc2-3)c2c(c1)C(C)(C)c1cc(C(C)(C)C)ccc1-2 ; CC(C)(C)c1ccc2c(c1)C(C)(C)c1cc(C(C)(C)C)cc(N(c3ccc4c(c3)C(C)(C)c3ccccc3-4)c3cccc4oc5ccccc5c34)c1-2 ; CC(C)(C)c1ccc2c(c1)C(C)(C)c1cc(C(C)(C)C)c(N(c3ccc(-c4ccccc4)cc3)c3ccc4c(c3)C(C)(C)c3ccccc3-4)cc1-2]

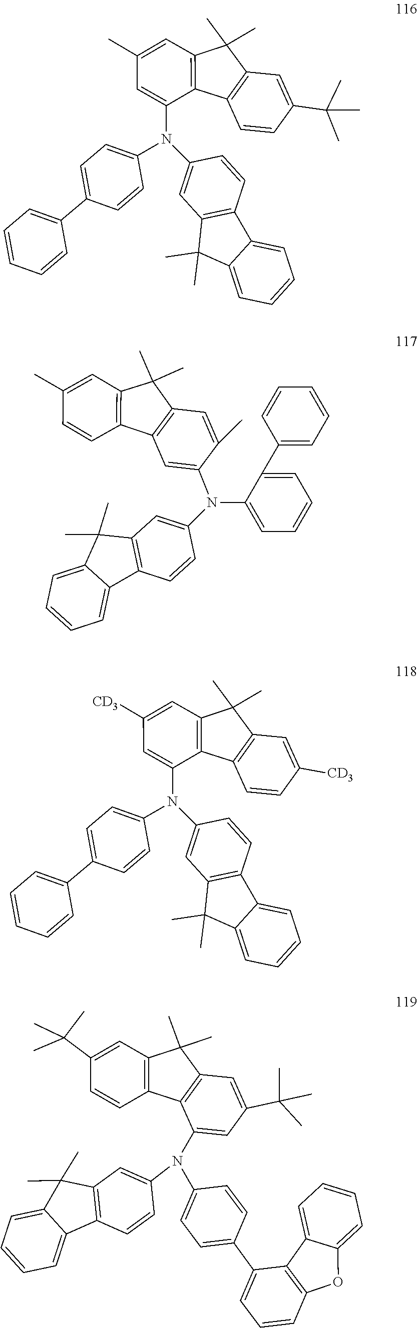

The present application further provides the following compounds:

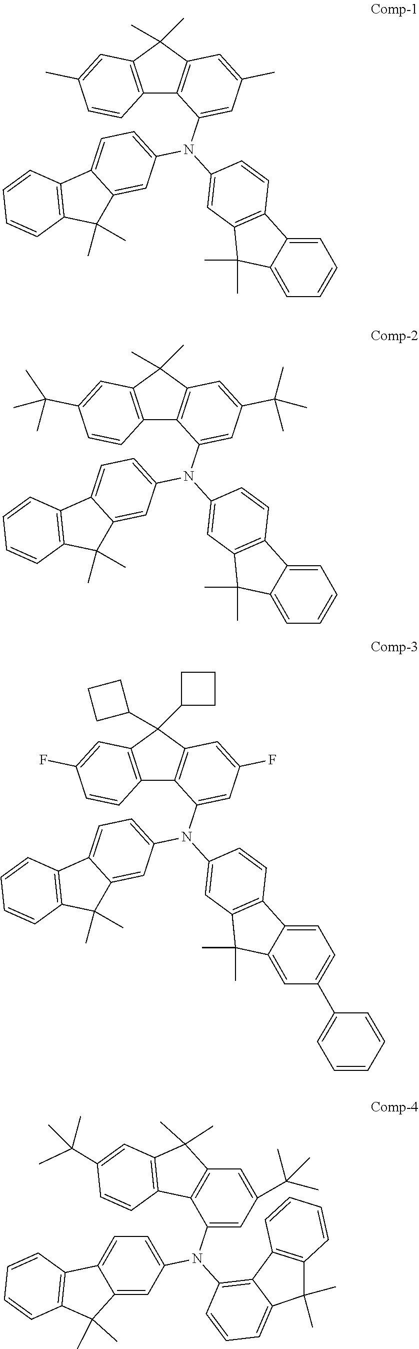 [SMILES: Cc1ccc2c(c1)C(C)(C)c1cc(C)cc(N(c3ccc4c(c3)C(C)(C)c3ccccc3-4)c3ccc4c(c3)C(C)(C)c3ccccc3-4)c1-2 ; CC(C)(C)c1ccc2c(c1)C(C)(C)c1cc(C(C)(C)C)cc(N(c3ccc4c(c3)C(C)(C)c3ccccc3-4)c3ccc4c(c3)C(C)(C)c3ccccc3-4)c1-2 ; CC1(C)c2ccccc2-c2ccc(N(c3ccc4c(c3)C(C)(C)c3cc(-c5ccccc5)ccc3-4)c3cc(F)cc4c3-c3ccc(F)cc3C4(C3CCC3)C3CCC3)cc21 ; CC(C)(C)c1ccc2c(c1)C(C)(C)c1cc(C(C)(C)C)cc(N(c3ccc4c(c3)C(C)(C)c3ccccc3-4)c3cccc4c3-c3ccccc3C4(C)C)c1-2]

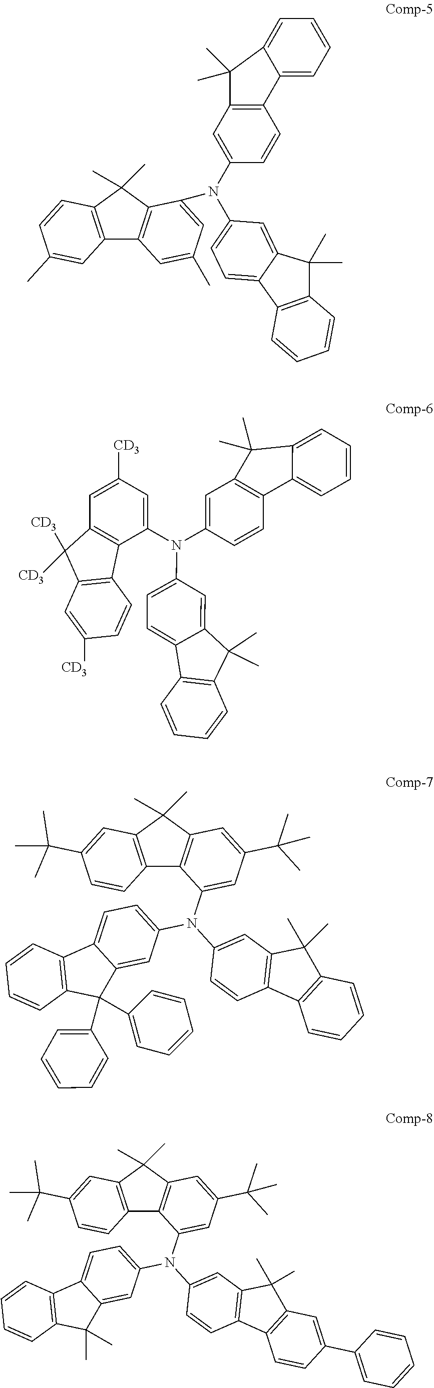

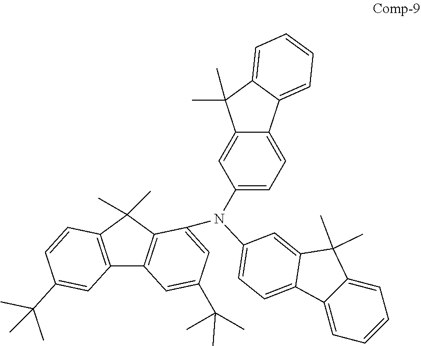 [SMILES: CC(C)(C)c1ccc2c(c1)-c1cc(C(C)(C)C)cc(N(c3ccc4c(c3)C(C)(C)c3ccccc3-4)c3ccc4c(c3)C(C)(C)c3ccccc3-4)c1C2(C)C]

The compounds according to the application can be prepared by the person skilled in the art by means of known organic chemistry reactions.

In a preferred process for preparing the compounds, in a first step (scheme 1A), in a Suzuki reaction between a phenylboronic acid derivative and a carboxylic ester-substituted and dihalogen-substituted phenyl derivative, a biphenyl derivative is prepared, bearing one halogen group and one carboxylic ester group.

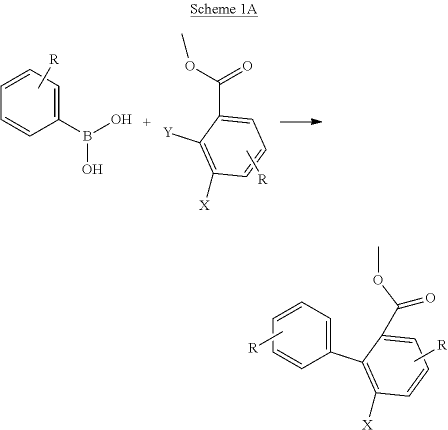

X and Y here are selected from reactive groups, preferably halogen atoms, more preferably Cl, Br and I. R is the same or different at each instance and is selected from H, D and organic radicals that are preferably selected from alkyl groups, aromatic ring systems and heteroaromatic ring systems. Rather than one radical, it is also possible for two or more R radicals each to be bonded to one benzene ring.

In a second step, reaction is effected with alkyl Grignard reagent or alkyllithium, and this is followed by an acid-catalyzed ring closure reaction in which a halogen-substituted fluorenyl derivative is formed. If starting substances containing the halogen atom in a corresponding different position are used in scheme 1A, it is correspondingly possible to obtain, rather than the 4-halogen-substituted fluorene derivative shown in scheme 2A, fluorene derivatives substituted in the 1 or 3 position.

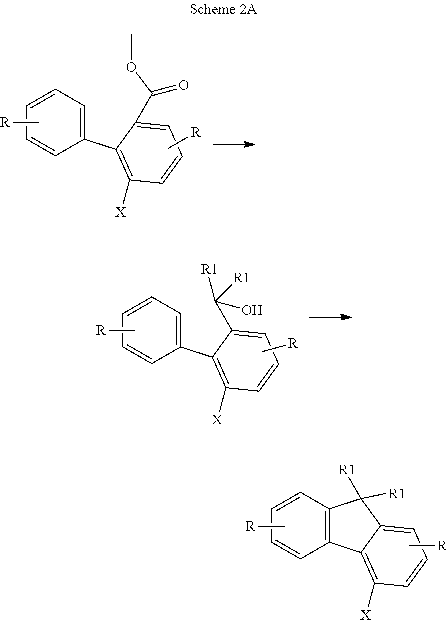

The halogen-substituted fluorene derivatives may alternatively be prepared by the following process: In a first step, as shown in scheme 1B, a biphenyl derivative substituted by two reactive groups, preferably two halogen atoms, is prepared via a Suzuki reaction.

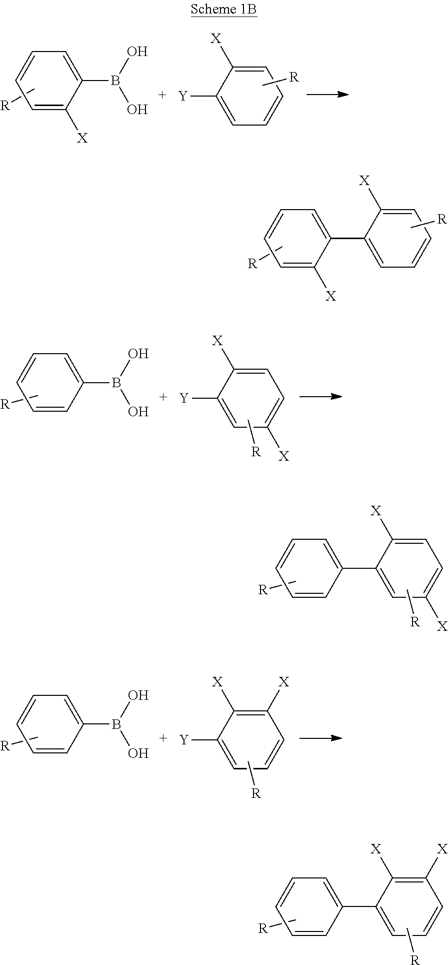

The variable groups are as defined above.

In a second step, as shown in scheme 2B, the biphenyl derivative obtained, bearing two reactive groups, especially two halogen atoms, is reacted with a carbonyl derivative and a metal organyl, especially BuLi. The resulting intermediate is converted under acidic conditions (H′) to a fluorenyl derivative. Depending on the position of the reactive groups, what is obtained is a fluorenyl derivative having the reactive group in the 1, 3 or 4 position.

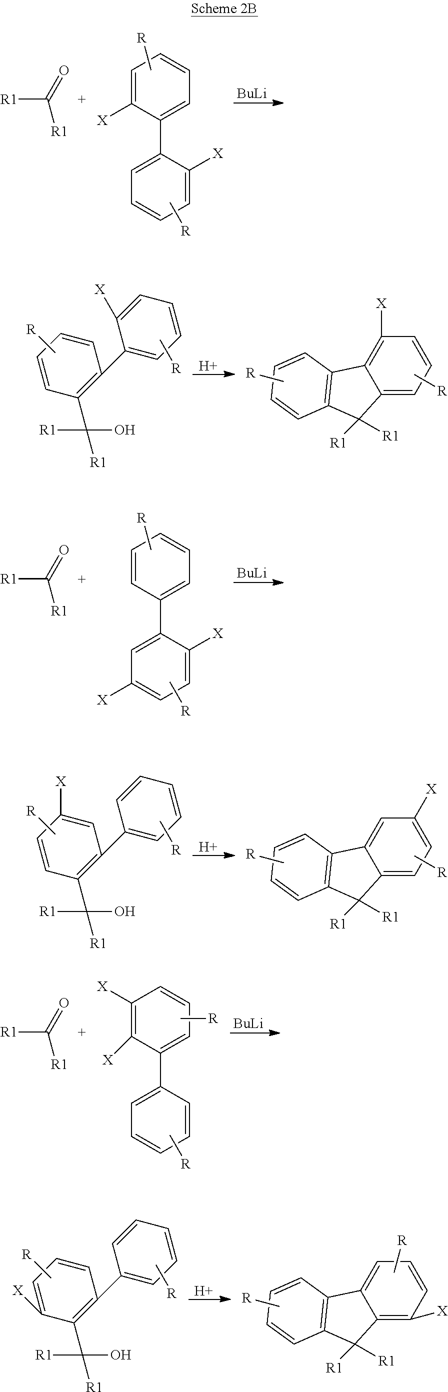

The variable groups are as defined above, and R1 is an organic radical, preferably an alkyl group.

The fluorenyl derivative obtained can be converted to a compound according to the application by several routes. By the route shown in scheme 3, the fluorenyl derivative is reacted with a secondary amine in a Buchwald reaction. From the top downward, the scheme shows the respective 4, 1 and 3 positions of the amine on the fluorene.

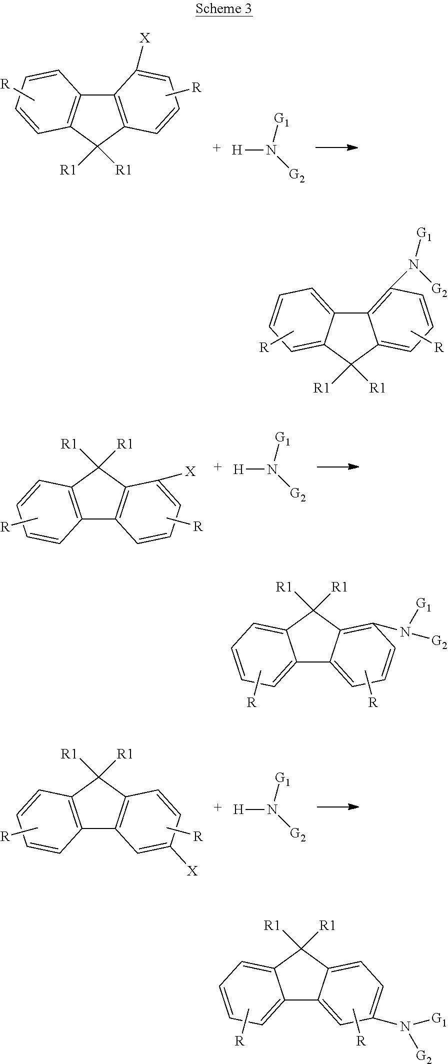

G₁ and G₂ here are selected from organic radicals, especially aromatic ring systems and heteroaromatic ring systems, and the other variable groups are as defined above.

Alternatively, the fluorenyl derivative can be reacted by the route shown in scheme 4 in a Suzuki reaction with a boronic acid-substituted tri(het)arylamine. From the top downward, the scheme shows the respective 4, 1 and 3 positions of the amine on the fluorene.

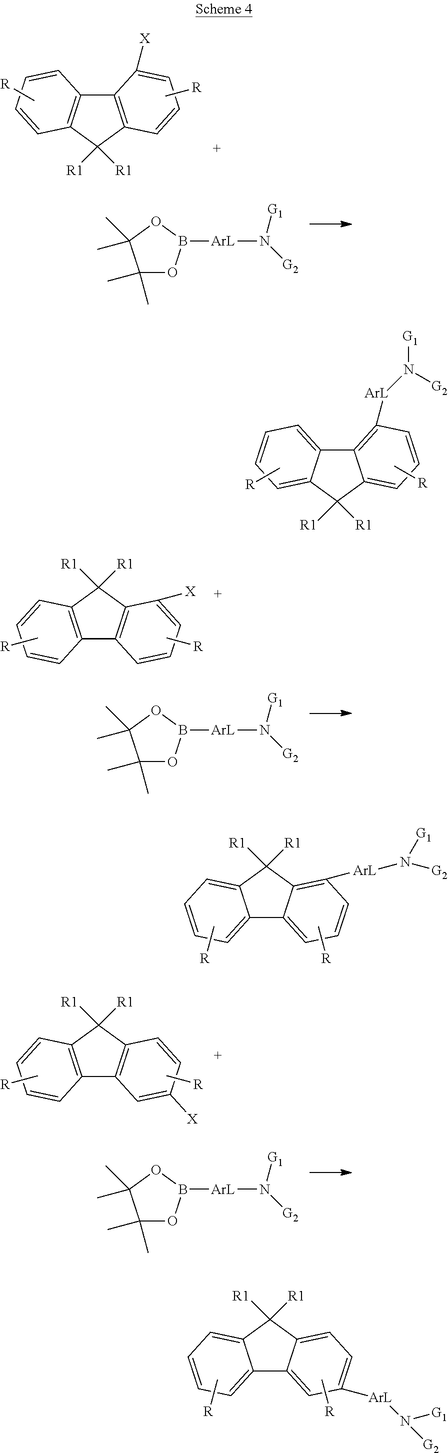

ArL here is selected from aromatic ring systems and heteroaromatic ring systems, and the other variable groups are as defined above.

Finally, the compound according to the application can also be prepared by the route shown in scheme 5, in which there is firstly a Suzuki coupling with a suitably substituted aromatic heteroaromatic system, and the resultant coupled compound is then reacted in a Buchwald reaction with a secondary amine. From the top downward, the scheme shows the respective 4, 1 and 3 positions of the amine on the fluorene.

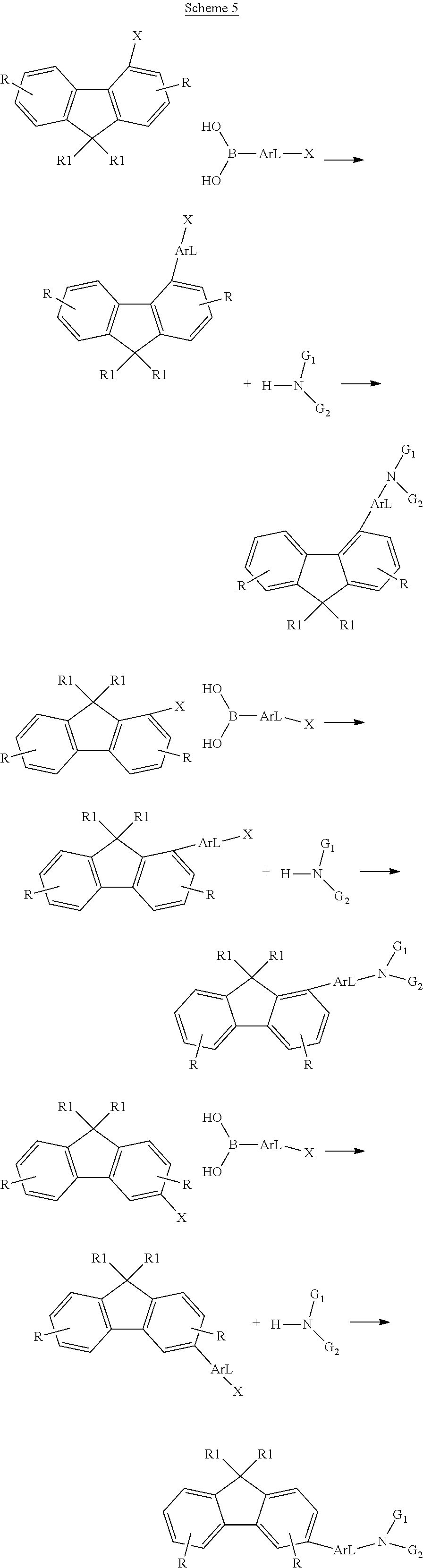

The variable groups are as defined above.

The person skilled in the art, in the preparation of the compounds according to the application, is not restricted to the synthesis methods specified above, but will be able within the scope of their common art knowledge to use other synthesis routes and/or to modify the abovementioned synthesis routes.

The present application provides a process for preparing a compound of the formula (I), characterized in that a fluorenyl compound bearing at least one reactive group is either a) reacted with a secondary amine in a Buchwald reaction, or b) reacted with a boronic acid-substituted tertiary amine in a Suzuki reaction, or c) reacted in a sequence of first i) Suzuki reaction with a boronic acid-substituted and halogen-substituted aromatic or heteroaromatic compound, followed by ii) Buchwald reaction of the resultant intermediate with a secondary amine, to give a compound of the formula (I).

The reactive group is preferably selected from Cl, Br and I.

The abovementioned fluorenyl compound bearing at least one reactive group is preferably prepared by reacting a halogen-substituted biphenyl compound with a carbonyl derivative, preferably a dialkylcarbonyl derivative and a metal organyl, preferably BuLi.

In an alternative, likewise preferred process, the abovementioned fluorenyl compound bearing at least one reactive group is obtained by reacting a biphenyl compound bearing a carboxylic ester group with Grignard reagent.

For the processing of the compounds according to the application from a liquid phase, for example by spin-coating or by printing methods, formulations of the compounds according to the application are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (—)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound according to the application and at least one solvent, preferably an organic solvent. The way in which such solutions can be prepared is known to those skilled in the art.

The compound according to the application is suitable for use in an electronic device, especially an organic electroluminescent device (OLED). Depending on the substitution, the compound according to the application can be used in different functions and layers. Preference is given to use as a hole-transporting material in a hole-transporting layer and/or as matrix material in an emitting layer, more preferably in combination with a phosphorescent emitter.

The invention therefore further provides for the use of a compound according to the application in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound according to the application. This electronic device is preferably selected from the abovementioned devices.

Particular preference is given to an organic electroluminescent device comprising an anode, cathode and at least one emitting layer, characterized in that at least one organic layer comprising at least one compound according to the application is present in the device. Preference is given to an organic electroluminescent device comprising an anode, cathode and at least one emitting layer, characterized in that at least one organic layer in the device, selected from hole-transporting and emitting layers, comprises at least one compound according to the application.

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
—anode—
—hole injection layer—
—hole transport layer—
—optionally further hole transport layers—
—emitting layer—
—optionally hole blocker layer—
—electron transport layer—
—electron injection layer—
—cathode—.

At the same time, it should be pointed out again that not all the layers mentioned need be present and/or that further layers may additionally be present.

In a preferred embodiment, the electronic device containing the compound according to the application contains multiple emitting layers arranged in succession, each having different emission maxima between 380 nm and 750 nm. In other words, different emitting compounds used in each of the multiple emitting layers fluoresce or phosphoresce and emit blue, green, yellow, orange or red light. In a preferred embodiment, the electronic device contains three emitting layers in succession in a stack, of which one in each case exhibits blue emission, one green emission, and one orange or red, preferably red, emission. Preferably, in this case, the blue-emitting layer is a fluorescent layer, and the green-emitting layer is a phosphorescent layer, and the red- or orange-emitting layer is a phosphorescent layer. The compound according to the application here is preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound according to the application is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound according to the application contains a phosphorescent emitting layer, it is preferable that this layer contains two or more, preferably exactly two, different matrix materials (mixed matrix system). Preferred embodiments of mixed matrix systems are described in detail further down.

If the compound according to the application is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound according to the application additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound according to the application and the one or more further hole-transporting compounds are preferably each present in a proportion of at least 10%, more preferably each in a proportion of at least 20%.

In a preferred embodiment, a hole-transporting layer comprising the compound according to the application additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, 12, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site.

Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (Ill) oxidation state, more particularly bismuth(Ill) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Especially preferred p-dopants are the compounds shown in the table on page 99 to page 100 of WO2021/104749.

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound according to the application may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

More preferably, the compound according to the application is present in an electron blocker layer. In this case, it is preferably not p-doped. Further preferably, in this case, it is preferably in the form of a single compound in the layer without addition of a further compound.

In an alternative preferred embodiment, the compound according to the application is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds according to the application are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound according to the application in a mixed matrix system is preferably the matrix material having hole-transporting properties. Correspondingly, when the compound according to the application is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

### Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. The compounds depicted in the following table are especially suitable:

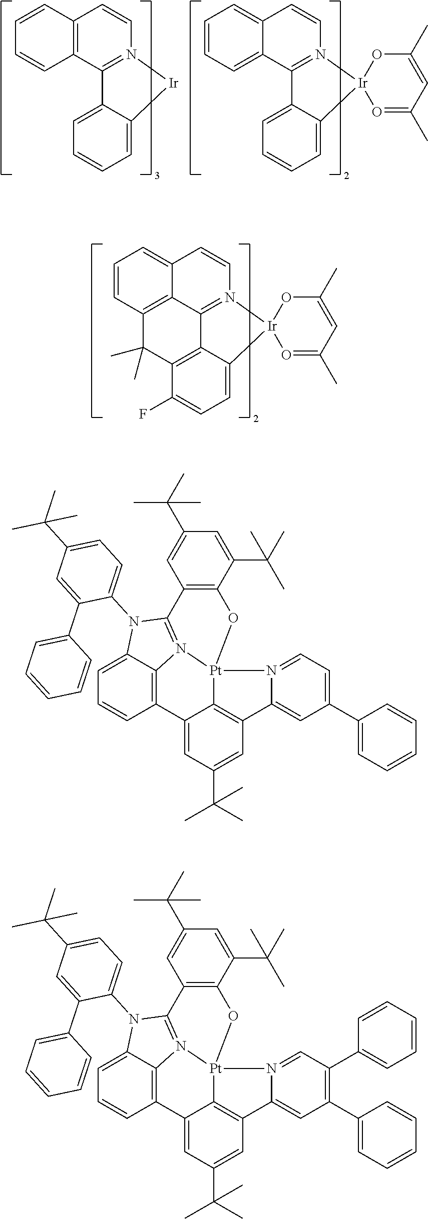

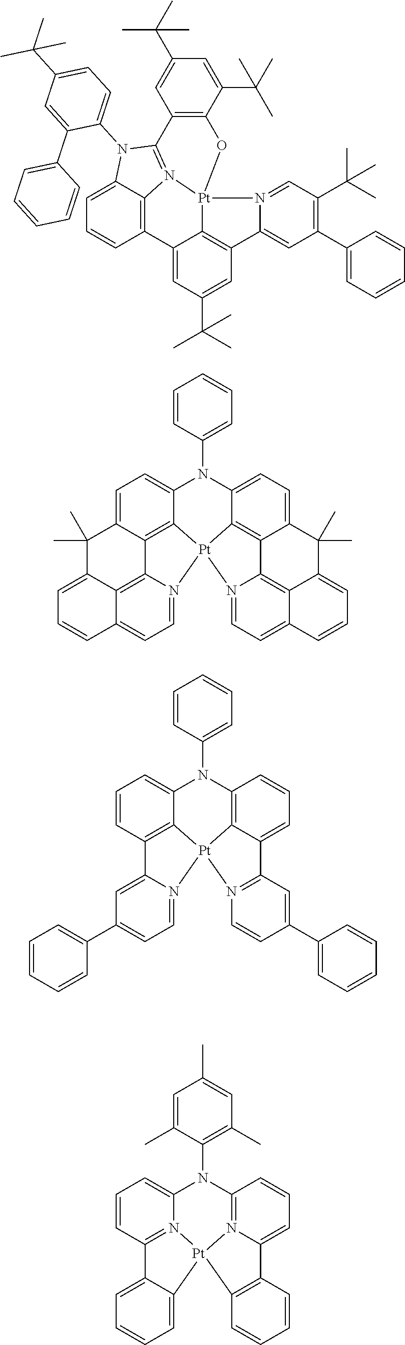

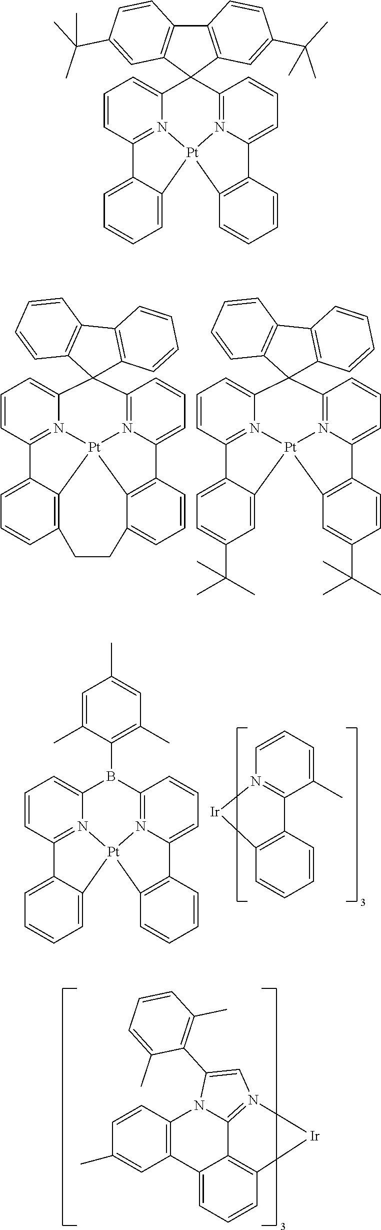

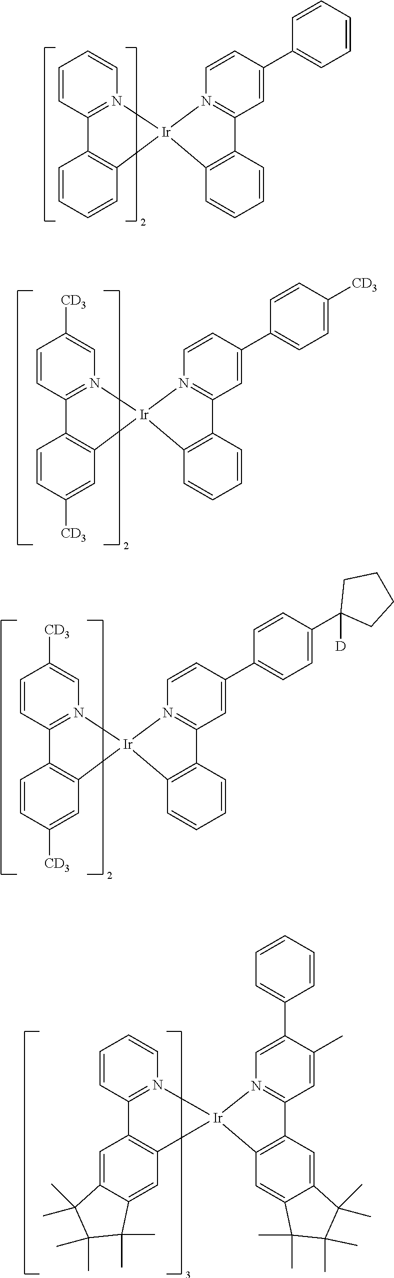

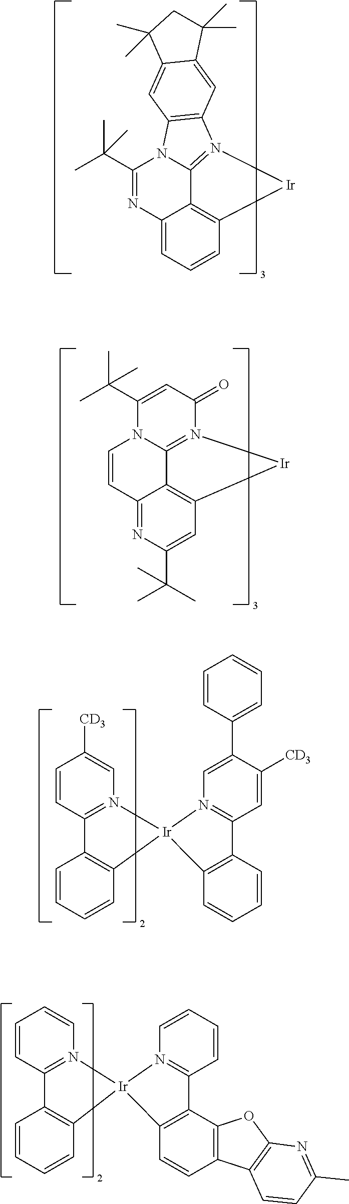

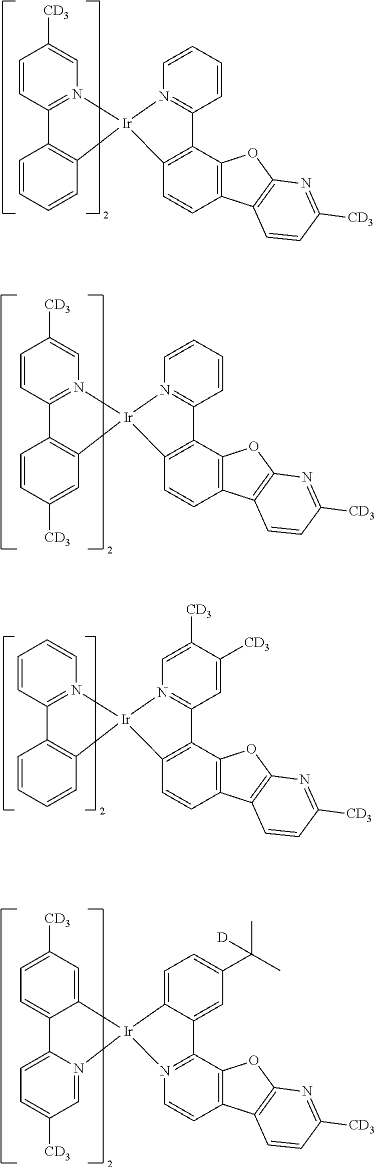

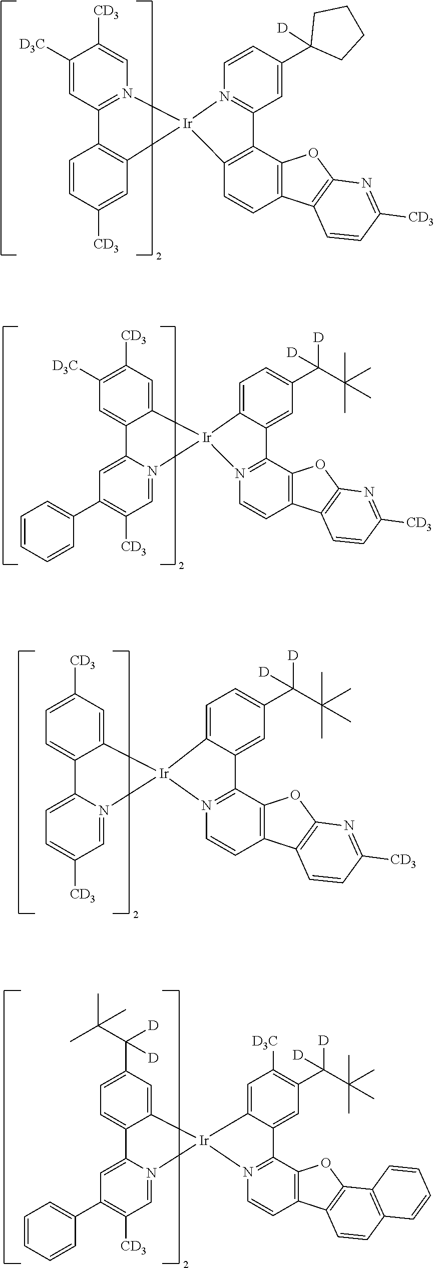

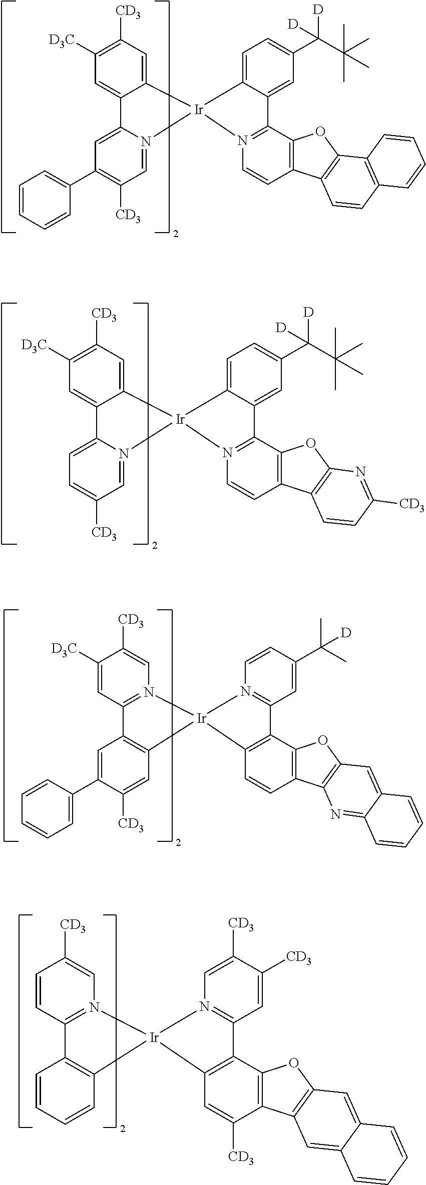

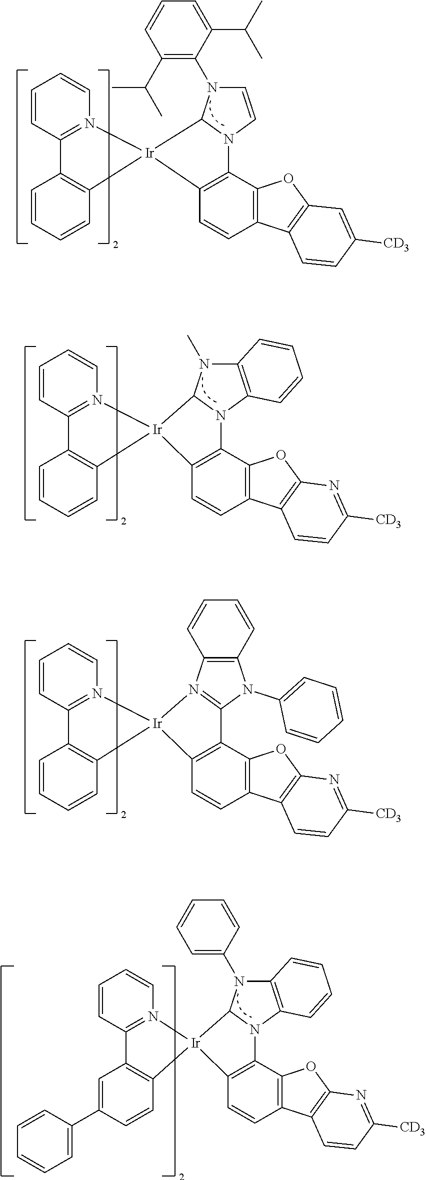

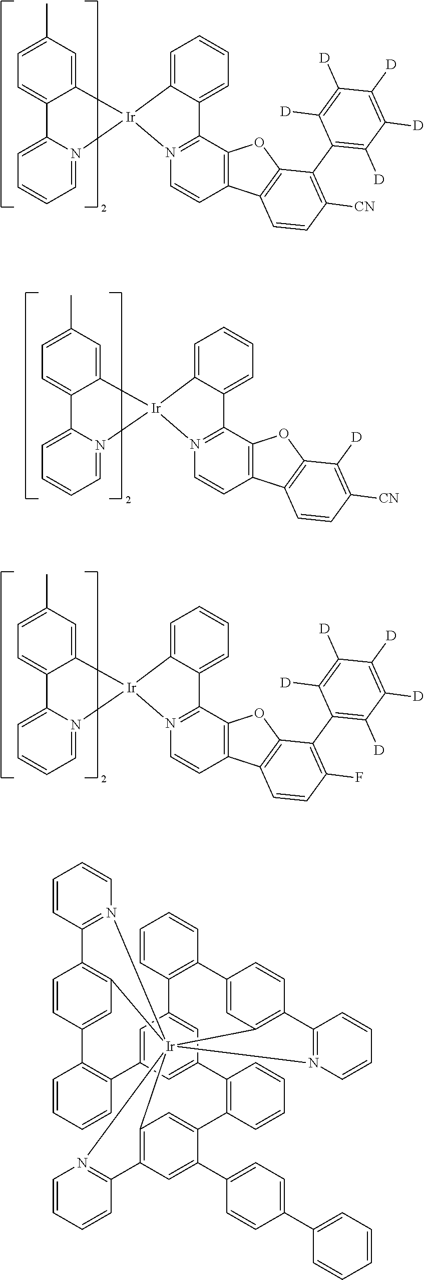

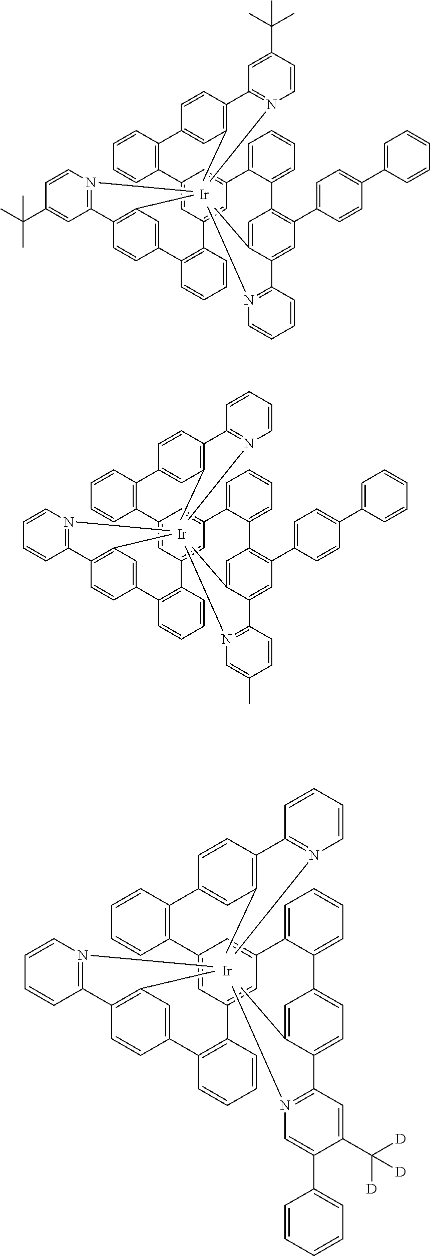

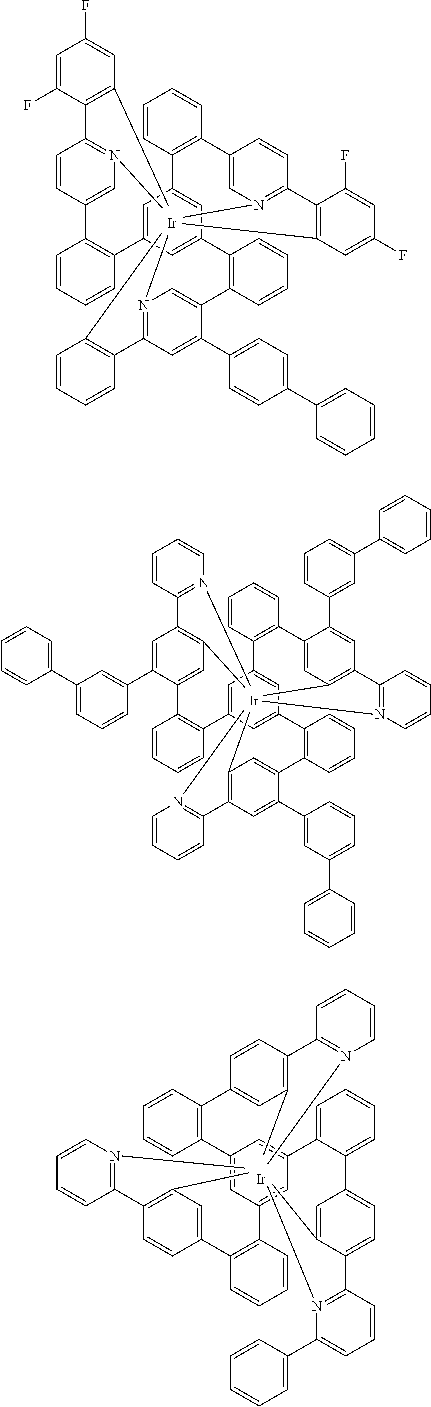

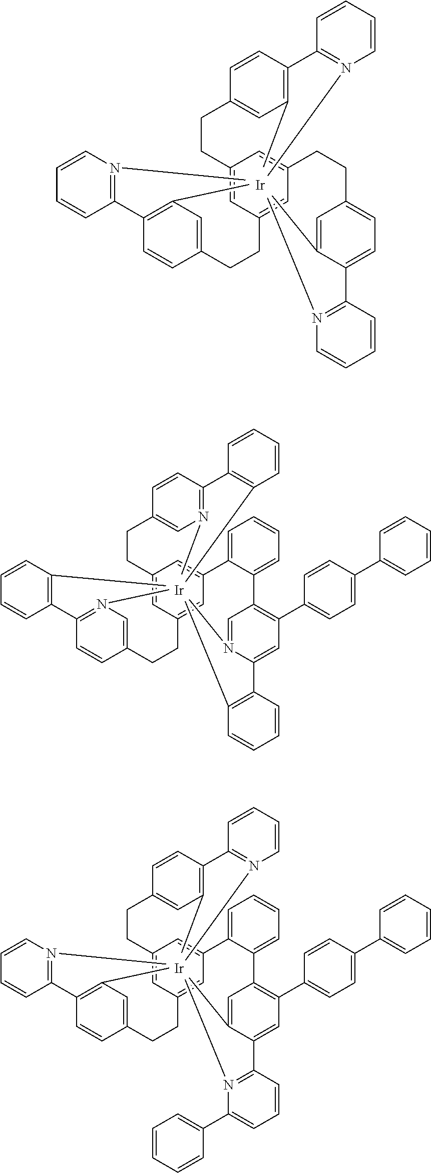

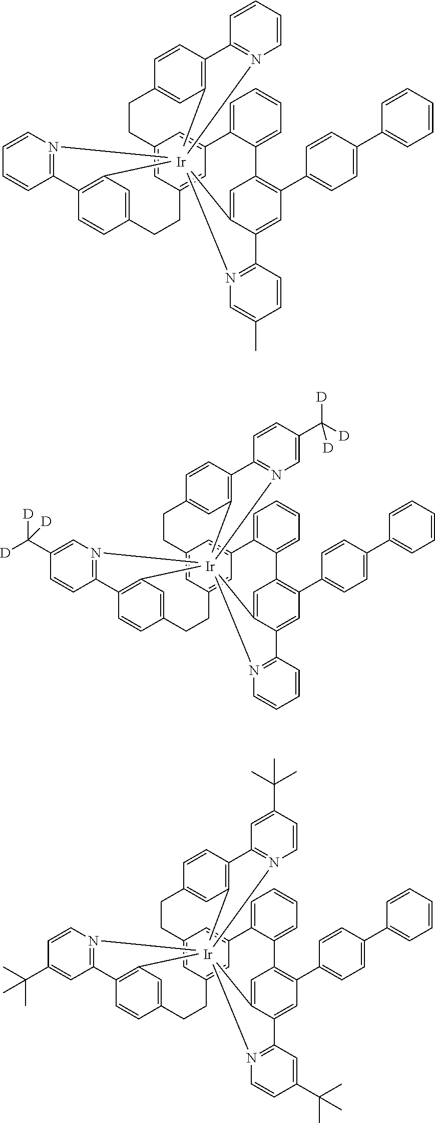

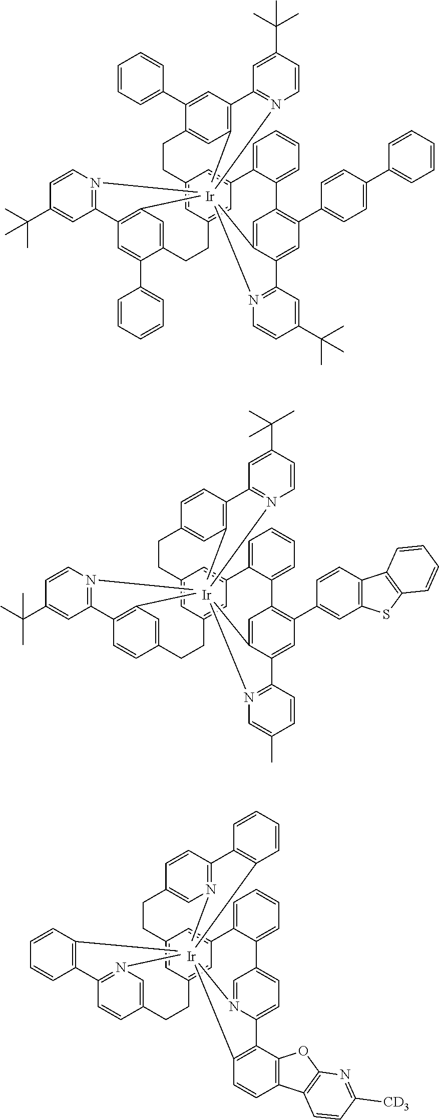

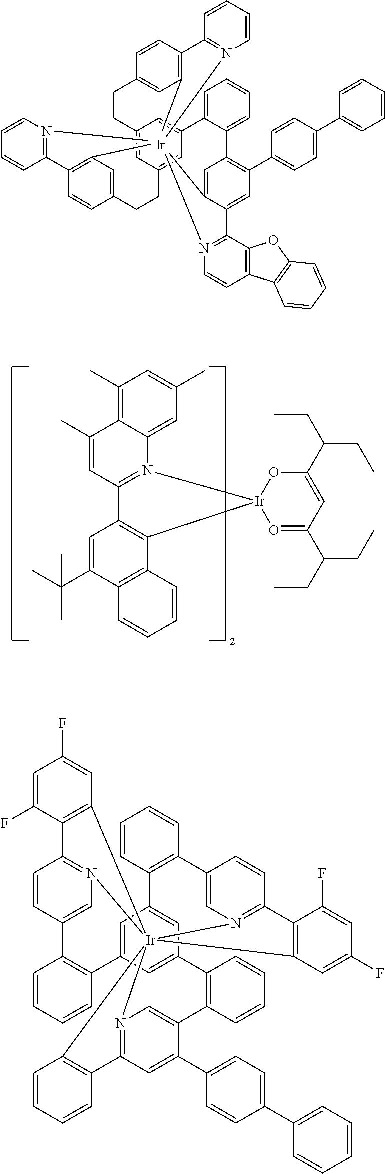

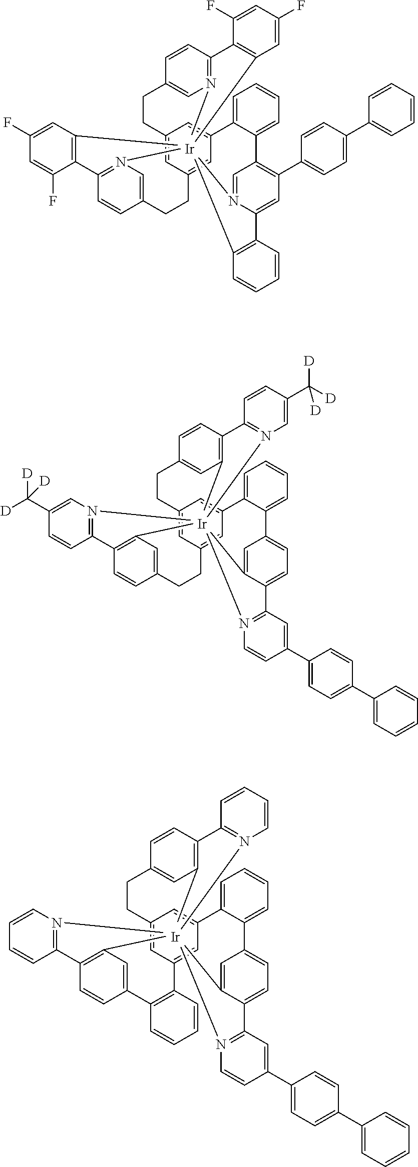

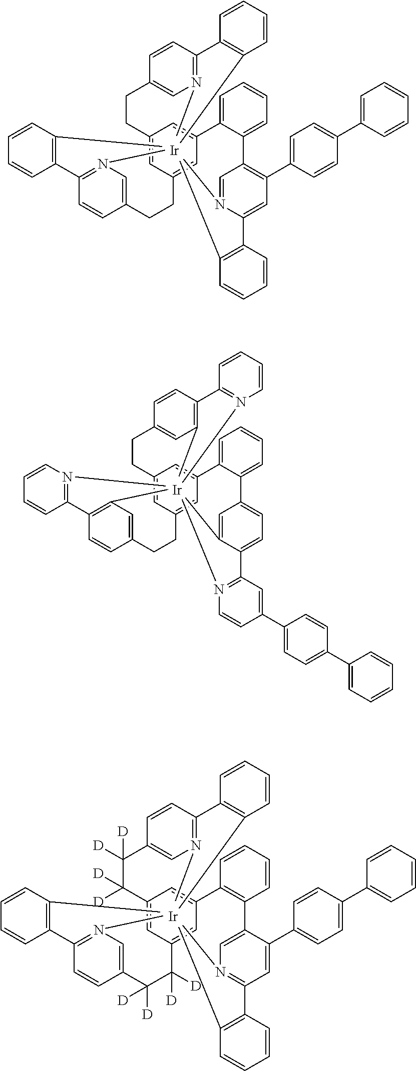

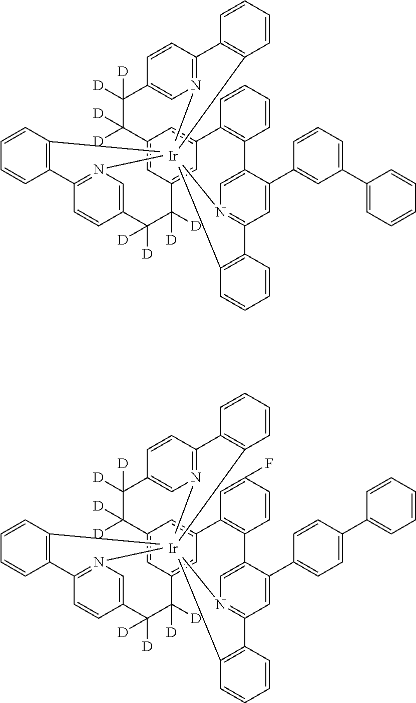

### Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Matrix materials for fluorescent emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Matrix materials for phosphorescent emitters:

Preferred matrix materials for phosphorescent emitters are, as well as the compounds according to the application, aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Electron-transporting materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are the compounds shown in the table on page 122 to page 123 of WO2020/127176.

Hole-transporting materials:

Further compounds which, in addition to the compounds according to the application, are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are especially the compounds disclosed in the table from the bottom of page 116 to the bottom of page 120 in WO 2021/104749.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. AI/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds according to the application are needed. High solubility can be achieved by suitable substitution of the compounds.

It is further preferable that an electronic device of the invention is produced by applylng one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds according to the application can be used in displays, as light sources in lighting applications and as light sources in medical and/or cosmetic applications.

### EXAMPLES

### A) Synthesis Examples

### Synthesis of 2-bromo-4-tert-butyl-2′-chloro-4′-methyl-1,1′-biphenyl 1a

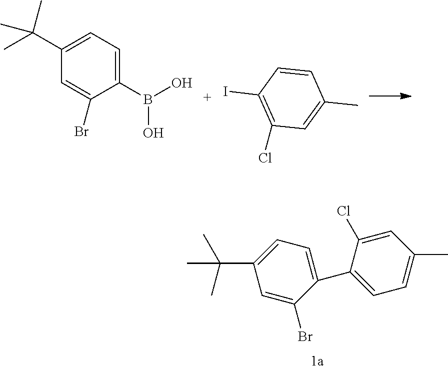

8.8 g (34.2 mmol) of (2-bromo-4-tert-butylphenyl)boronic acid and 12.9 g (51.3 mmol) of methyl 2-chloro-1-iodo-4-methylbenzene are suspended in 200 ml of THE and 38 ml of a 2M potassium carbonate solution (75.5 mmol). 0.87 g (0.76 mmol) of tetrakis(triphenylphosphine)palladium is added to this suspension, and the reaction mixture is heated under reflux for 12 h. After cooling, the organic phase is removed, filtered through silica gel, washed three times with 100 ml of water and then concentrated to dryness. After the crude product has been filtered through silica gel with toluene, 10.9 g (95%) of 1a is obtained.

The following compounds are prepared in an analogous manner:

| | Halide | Boronic acid | Product |
|---|---|---|---|
| 1b | 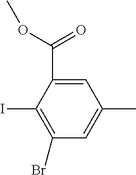 | 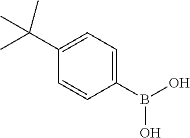 | 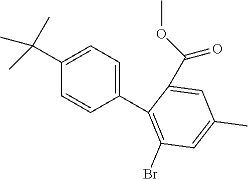 [SMILES: COC(=O)c1cc(C)cc(Br)c1-c1ccc(C(C)(C)C)cc1] |
| 1c | 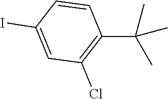 | 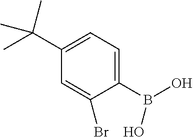 | 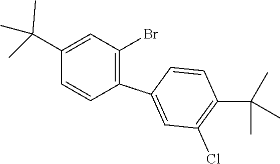 [SMILES: CC(C)(C)c1ccc(-c2ccc(C(C)(C)C)c(Cl)c2)c(Br)c1] |
| 1d | 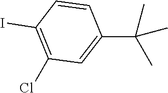 | 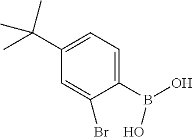 | 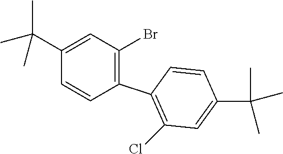 [SMILES: CC(C)(C)c1ccc(-c2ccc(C(C)(C)C)cc2Br)c(Cl)c1] |
| 1f | 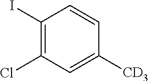 [SMILES: [2H]C([2H])([2H])c1ccc(I)c(Cl)c1] | 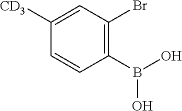 [SMILES: [2H]C([2H])([2H])c1ccc(B(O)O)c(Br)c1] | 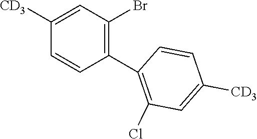 [SMILES: [2H]C([2H])([2H])c1ccc(-c2ccc(C([2H])([2H])[2H])cc2Br)c(Cl)c1] |
| 1g | 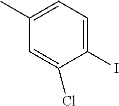 | 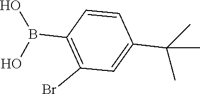 | 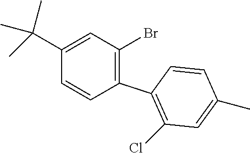 [SMILES: Cc1ccc(-c2ccc(C(C)(C)C)cc2Br)c(Cl)c1] |
| 1h | 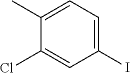 | 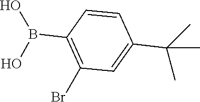 | 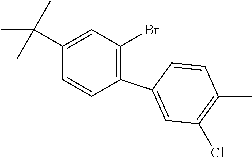 [SMILES: Cc1ccc(-c2ccc(C(C)(C)C)cc2Br)cc1Cl] |
| 1i | 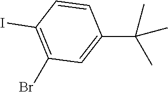 | 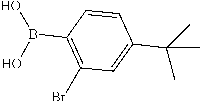 | 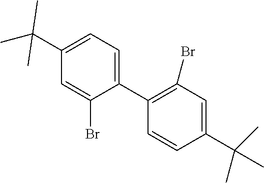 [SMILES: CC(C)(C)c1ccc(-c2ccc(C(C)(C)C)cc2Br)c(Br)c1] |
| 1j | 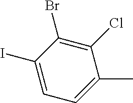 | 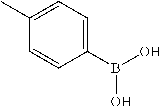 | 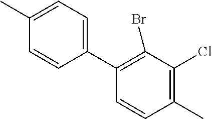 [SMILES: Cc1ccc(-c2ccc(C)c(Cl)c2Br)cc1] |
| 1k | 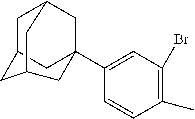 [SMILES: Cc1ccc(C23CC4CC(CC(C4)C2)C3)cc1Br] | 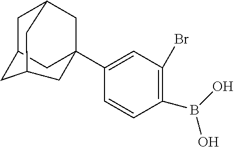 [SMILES: OB(O)c1ccc(C23CC4CC(CC(C4)C2)C3)cc1Br] | 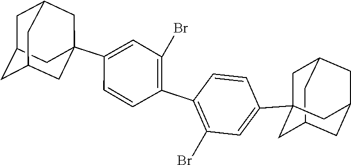 [SMILES: Brc1cc(C23CC4CC(CC(C4)C2)C3)ccc1-c1ccc(C23CC4CC(CC(C4)C2)C3)cc1Br] |
| 1l | 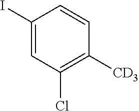 [SMILES: [2H]C([2H])([2H])c1ccc(I)cc1Cl] | 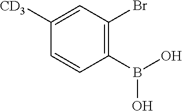 [SMILES: [2H]C([2H])([2H])c1ccc(B(O)O)c(Br)c1] | 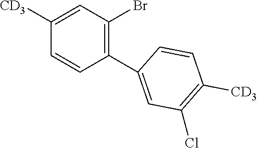 [SMILES: [2H]C([2H])([2H])c1ccc(-c2ccc(C([2H])([2H])[2H])c(Cl)c2)c(Br)c1] |
| 1m | 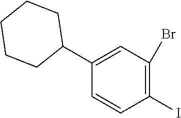 [SMILES: Brc1cc(C2CCCCC2)ccc1I] | 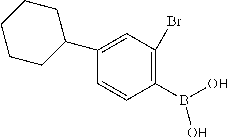 [SMILES: OB(O)c1ccc(C2CCCCC2)cc1Br] | 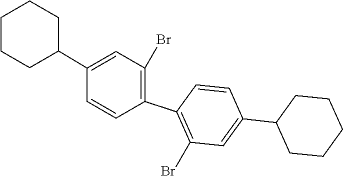 [SMILES: Brc1cc(C2CCCCC2)ccc1-c1ccc(C2CCCCC2)cc1Br] |

### Synthesis of 4-bromo-7-tert-butyl-2,9,9-trimethyl-9H-fluorene 2a

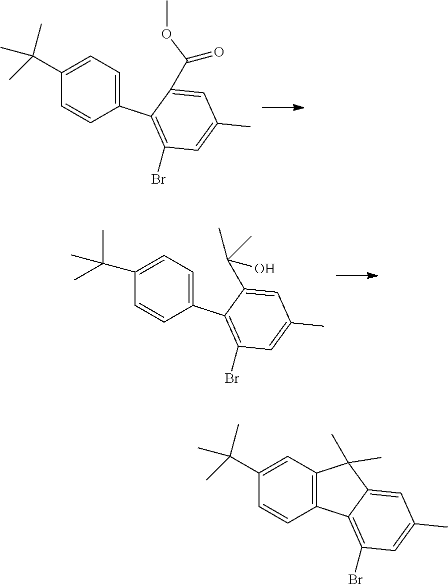

20 g (55.4 mmol) of methyl 6-bromo-4′-tert-butyl-4-methyl-[1,1′-biphenyl]-2-carboxylate is dissolved in 160 ml of tetrahydrofuran and cooled to −15° C., and 65.9 ml (198 mmol) of 3.0M methylmagnesium chloride in THE is slowly added dropwise. The reaction mixture is allowed to come to room temperature overnight. Water is added gradually to the mixture, then it is partitioned between EtOAc and water, and the organic phase is washed three times with water and dried over Na₂SO₄ and concentrated by rotary evaporation (14.6 g of pale yellow oil, 73% yleld).

2-{6-Bromo-4′-tert-butyl-4-methyl-[1,1′-biphenyl]-2-yl}propan-2-ol (14.6 g, 40.4 mmol) is dissolved in toluene (150 ml), 1.9 ml (2 mmol) of sulfuric acid is added and the mixture is stirred for 1 h. Water is added gradually to the mixture, then it is partitioned between EtOAc and water, and the organic phase is washed with NaHCO₃ and dried over Na₂SO₄ and concentrated by rotary evaporation. After the crude product has been filtered through silica gel with heptane, 10.9 g of 2a is isolated (79% yleld).

The following compounds are prepared in an analogous manner:

| Ex. | Ester | Product |
|---|---|---|
| 2b | 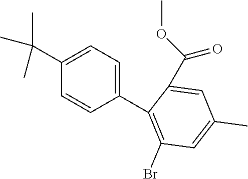 [SMILES: COC(=O)c1cc(C)cc(Br)c1-c1ccc(C(C)(C)C)cc1] | 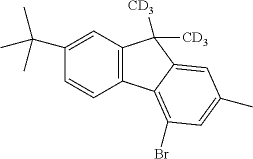 [SMILES: [2H]C([2H])([2H])C1(C([2H])([2H])[2H])c2cc(C(C)(C)C)ccc2-c2c(Br)cc(C)cc21] |
| 2c | 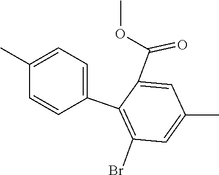 | 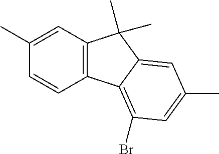 |
| 2d | 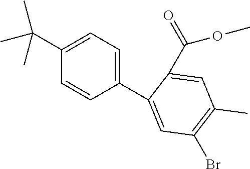 [SMILES: COC(=O)c1cc(C)c(Br)cc1-c1ccc(C(C)(C)C)cc1] | 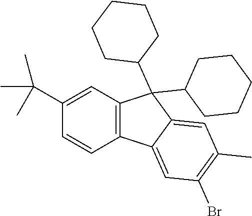 [SMILES: Cc1cc2c(cc1Br)-c1ccc(C(C)(C)C)cc1C2(C1CCCCC1)C1CCCCC1] |
| 2e | 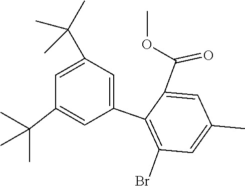 [SMILES: COC(=O)c1cc(C)cc(Br)c1-c1cc(C(C)(C)C)cc(C(C)(C)C)c1] | 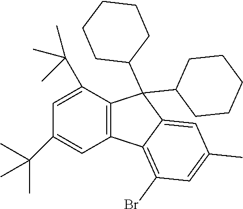 [SMILES: Cc1cc(Br)c2c(c1)C(C1CCCCC1)(C1CCCCC1)c1c-2cc(C(C)(C)C)cc1C(C)(C)C] |
| 2f | 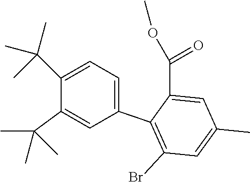 [SMILES: COC(=O)c1cc(C)cc(Br)c1-c1ccc(C(C)(C)C)c(C(C)(C)C)c1] | 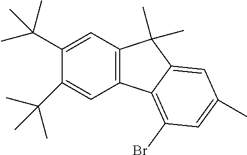 [SMILES: Cc1cc(Br)c2c(c1)C(C)(C)c1cc(C(C)(C)C)c(C(C)(C)C)cc1-2] |
| 2g | 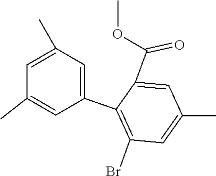 | 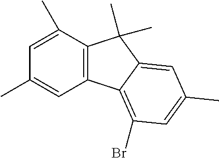 |
| 2h | 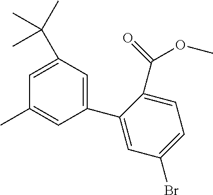 | 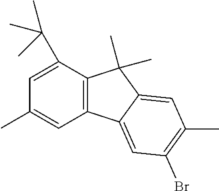 |
| 2i | 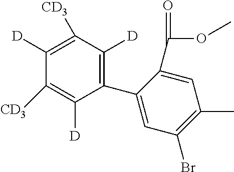 [SMILES: [2H]c1c(-c2cc(Br)c(C)cc2C(=O)OC)c([2H])c(C([2H])([2H])[2H])c([2H])c1C([2H])([2H])[2H]] | 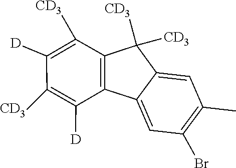 [SMILES: [2H]c1c2c(c(C([2H])([2H])[2H])c([2H])c1C([2H])([2H])[2H])C(C([2H])([2H])[2H])(C([2H])([2H])[2H])c1cc(C)c(Br)cc1-2] |
| 2j | 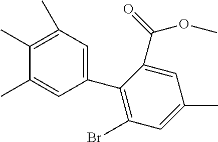 | 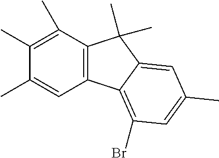 |

### Synthesis of 7-tert-butyl-4-chloro-2,9,9-trimethyl-9H-fluorene 3a

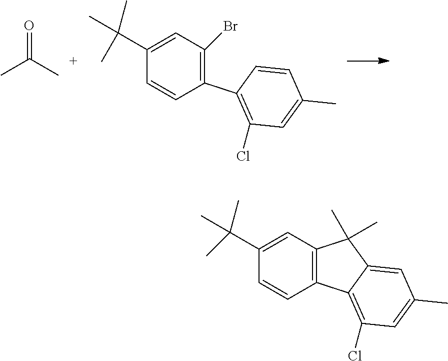

39.9 g (118 mmol) of 2-bromo-4′-tert-butyl-2′-chloro-4′-methyl-1,1′-biphenyl is dissolved in 300 ml of dried THE in a baked-out flask. The reaction mixture is cooled to −78° C. At this temperature, 39.3 ml of a 2.5 M solution of n-BuLi in hexane (98.2 mmol) is slowly added dropwise. The mixture is stirred at −70° C. for a further 1 hour. Subsequently, 17.9 g of propan-2-one (308.6 mmol) is dissolved in 300 ml of THE and added dropwise at −70° C. After the addition has ended, the reaction mixture is left to warm up gradually to room temperature, the reaction is stopped with NH₄Cl, and then the mixture is concentrated on a rotary evaporator. The solid matter is dissolved in 500 ml of toluene, and then 720 mg (3.8 mmol) of p-toluenesulfonic acid is added. The mixture is heated under reflux for 6 hours, then allowed to cool down to room temperature and admixed with water. The precipitated solids are filtered off with suction and washed with heptane (31.1 g, 88% yleld).

The following compounds are prepared in an analogous manner:

| Ex. | Biphenyl | Ketone | Product |
|---|---|---|---|
| 3b | 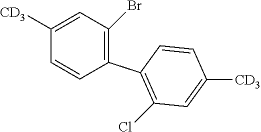 [SMILES: [2H]C([2H])([2H])c1ccc(-c2ccc(C([2H])([2H])[2H])cc2Br)c(Cl)c1] | 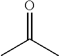 | 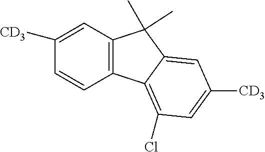 [SMILES: [2H]C([2H])([2H])c1ccc2c(c1)C(C)(C)c1cc(C([2H])([2H])[2H])cc(Cl)c1-2] |
| 3c | 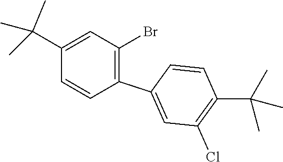 [SMILES: CC(C)(C)c1ccc(-c2ccc(C(C)(C)C)c(Cl)c2)c(Br)c1] | 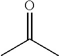 | 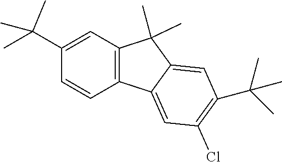 [SMILES: CC(C)(C)c1ccc2c(c1)C(C)(C)c1cc(C(C)(C)C)c(Cl)cc1-2] |
| 3d | 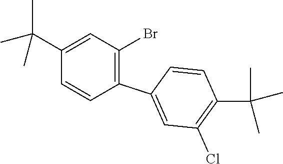 [SMILES: CC(C)(C)c1ccc(-c2ccc(C(C)(C)C)c(Cl)c2)c(Br)c1] | 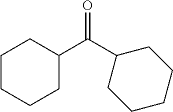 [SMILES: O=C(C1CCCCC1)C1CCCCC1] | 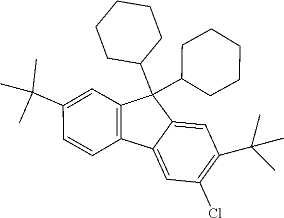 [SMILES: CC(C)(C)c1ccc2c(c1)C(C1CCCCC1)(C1CCCCC1)c1cc(C(C)(C)C)c(Cl)cc1-2] |
| 3e | 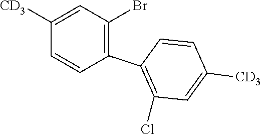 [SMILES: [2H]C([2H])([2H])c1ccc(-c2ccc(C([2H])([2H])[2H])cc2Br)c(Cl)c1] | 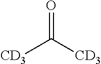 | 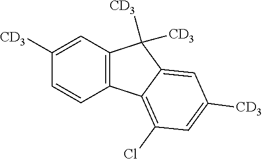 [SMILES: [2H]C([2H])([2H])c1ccc2c(c1)C(C([2H])([2H])[2H])(C([2H])([2H])[2H])c1cc(C([2H])([2H])[2H])cc(Cl)c1-2] |
| 3f | 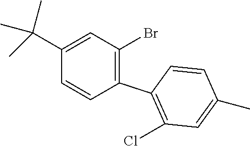 [SMILES: Cc1ccc(-c2ccc(C(C)(C)C)cc2Br)c(Cl)c1] | 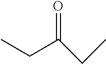 | 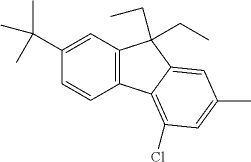 [SMILES: CCC1(CC)c2cc(C(C)(C)C)ccc2-c2c(Cl)cc(C)cc21] |
| 3g | 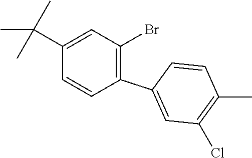 [SMILES: Cc1ccc(-c2ccc(C(C)(C)C)cc2Br)cc1Cl] | 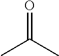 | 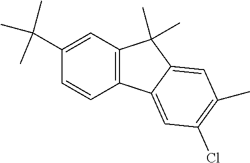 [SMILES: Cc1cc2c(cc1Cl)-c1ccc(C(C)(C)C)cc1C2(C)C] |
| 3h | 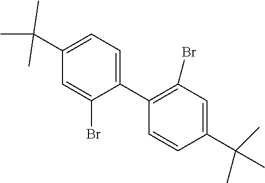 [SMILES: CC(C)(C)c1ccc(-c2ccc(C(C)(C)C)cc2Br)c(Br)c1] | 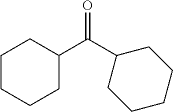 [SMILES: O=C(C1CCCCC1)C1CCCCC1] | 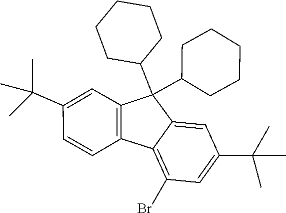 [SMILES: CC(C)(C)c1ccc2c(c1)C(C1CCCCC1)(C1CCCCC1)c1cc(C(C)(C)C)cc(Br)c1-2] |
| 3i | 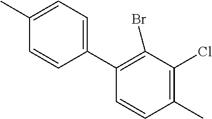 [SMILES: Cc1ccc(-c2ccc(C)c(Cl)c2Br)cc1] | 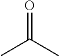 | 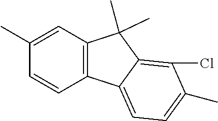 |
| 3j | 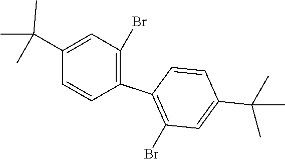 [SMILES: CC(C)(C)c1ccc(-c2ccc(C(C)(C)C)cc2Br)c(Br)c1] | 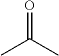 | 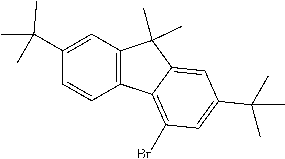 [SMILES: CC(C)(C)c1ccc2c(c1)C(C)(C)c1cc(C(C)(C)C)cc(Br)c1-2] |

### Synthesis of N-{[1,1′-biphenyl]-4-yl}-7-tert-butyl-N-(9,9-dimethyl-9H-fluoren-2-yl)-2,9,9-trimethyl-9H-fluorene-4-amine 4a

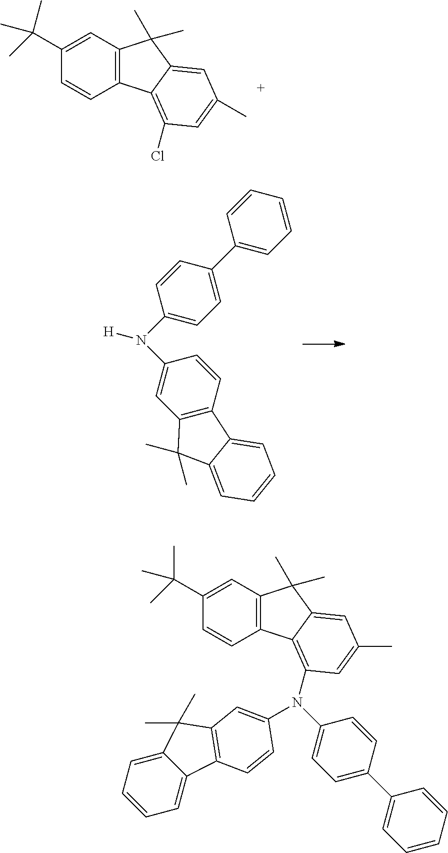 [SMILES: Cc1cc(Cl)c2c(c1)C(C)(C)c1cc(C(C)(C)C)ccc1-2 ; CC1(C)c2ccccc2-c2ccc(Nc3ccc(-c4ccccc4)cc3)cc21 ; Cc1cc(N(c2ccc(-c3ccccc3)cc2)c2ccc3c(c2)C(C)(C)c2ccccc2-3)c2c(c1)C(C)(C)c1cc(C(C)(C)C)ccc1-2]

10.9 g of N-{[1,1′-biphenyl]-4-yl}-9,9-dimethyl-9H-fluorene-2-amine (30.2 mmol) and 9.4 g of 7-tert-butyl-4-chloro-2,9,9-dimethyl-9H-fluorene (31.4 mol) are dissolved in 250 ml of toluene. The solution is degassed and saturated with N₂. It is subsequently admixed with 1 g (5.1 mmol) of S-Phos and 1.6 g (1.7 mmol) of Pd₂(dba)₃ and then 5 g of sodium tert-butoxide (52.05 mmol) is added. The reaction mixture is heated to boiling under a protective atmosphere overnight. The mixture is subsequently partitioned between toluene and water, and the organic phase is washed three times with water and dried over Na₂SO₄ and concentrated by rotary evaporation. After the crude product has been filtered through silica gel with toluene, the remaining residue is recrystallized from heptane/toluene. The substance is finally sublimed under high vacuum, purity is 99.9%. The yield is 7.1 g (38% of theory).

The following compounds are prepared in an analogous manner:

| Ex. | Halide | Amine |
|---|---|---|
| 4b | 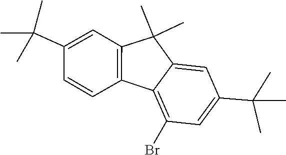 [SMILES: CC(C)(C)c1ccc2c(c1)C(C)(C)c1cc(C(C)(C)C)cc(Br)c1-2] | 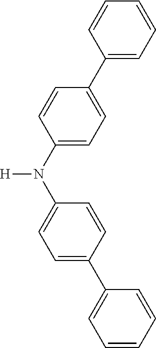 [SMILES: c1ccc(-c2ccc(Nc3ccc(-c4ccccc4)cc3)cc2)cc1] |
| 4c | 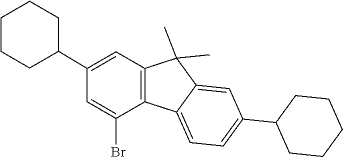 [SMILES: CC1(C)c2cc(C3CCCCC3)ccc2-c2c(Br)cc(C3CCCCC3)cc21] | 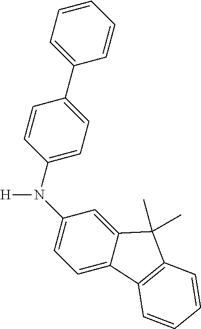 [SMILES: CC1(C)c2ccccc2-c2ccc(Nc3ccc(-c4ccccc4)cc3)cc21] |
| 4d | 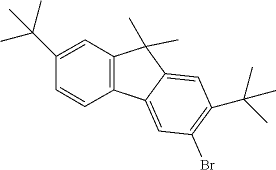 [SMILES: CC(C)(C)c1ccc2c(c1)C(C)(C)c1cc(C(C)(C)C)c(Br)cc1-2] | 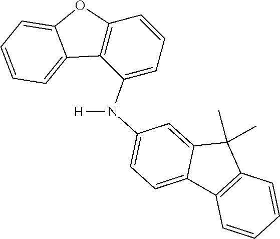 [SMILES: CC1(C)c2ccccc2-c2ccc(Nc3cccc4oc5ccccc5c34)cc21] |
| 4e | 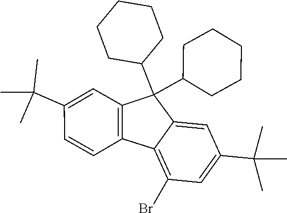 [SMILES: CC(C)(C)c1ccc2c(c1)C(C1CCCCC1)(C1CCCCC1)c1cc(C(C)(C)C)cc(Br)c1-2] | 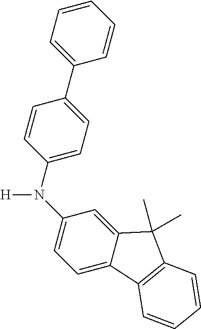 [SMILES: CC1(C)c2ccccc2-c2ccc(Nc3ccc(-c4ccccc4)cc3)cc21] |
| 4f | 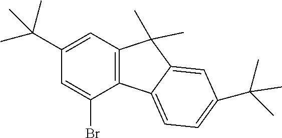 [SMILES: CC(C)(C)c1ccc2c(c1)C(C)(C)c1cc(C(C)(C)C)cc(Br)c1-2] | 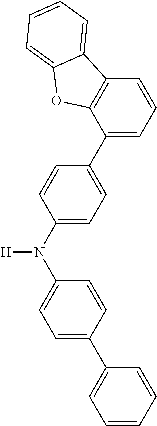 [SMILES: c1ccc(-c2ccc(Nc3ccc(-c4cccc5c4oc4ccccc45)cc3)cc2)cc1] |
| 4g | 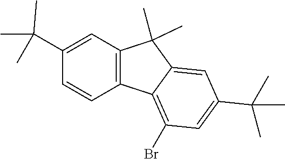 [SMILES: CC(C)(C)c1ccc2c(c1)C(C)(C)c1cc(C(C)(C)C)cc(Br)c1-2] | 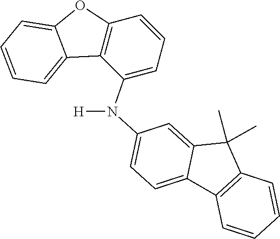 [SMILES: CC1(C)c2ccccc2-c2ccc(Nc3cccc4oc5ccccc5c34)cc21] |
| 4h | 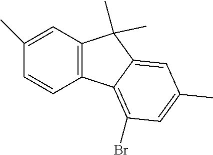 | 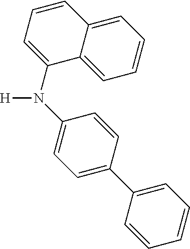 [SMILES: c1ccc(-c2ccc(Nc3cccc4ccccc34)cc2)cc1] |
| 4i | 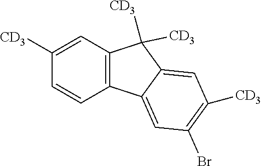 [SMILES: [2H]C([2H])([2H])c1ccc2c(c1)C(C([2H])([2H])[2H])(C([2H])([2H])[2H])c1cc(C([2H])([2H])[2H])c(Br)cc1-2] | 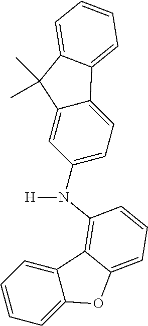 [SMILES: CC1(C)c2ccccc2-c2ccc(Nc3cccc4oc5ccccc5c34)cc21] |
| 4j | 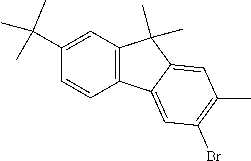 [SMILES: Cc1cc2c(cc1Br)-c1ccc(C(C)(C)C)cc1C2(C)C] | 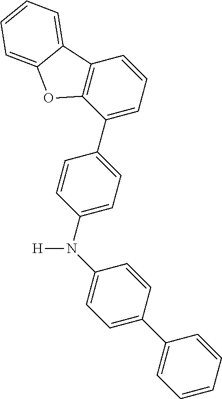 [SMILES: c1ccc(-c2ccc(Nc3ccc(-c4cccc5c4oc4ccccc45)cc3)cc2)cc1] |
| 4k | 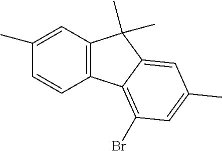 | 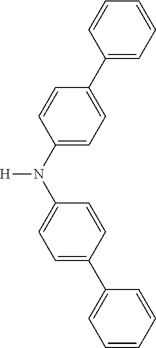 [SMILES: c1ccc(-c2ccc(Nc3ccc(-c4ccccc4)cc3)cc2)cc1] |
| 4l | 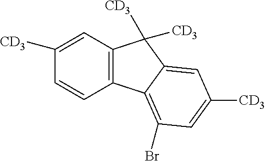 [SMILES: [2H]C([2H])([2H])c1ccc2c(c1)C(C([2H])([2H])[2H])(C([2H])([2H])[2H])c1cc(C([2H])([2H])[2H])cc(Br)c1-2] | 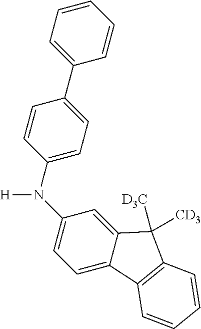 [SMILES: [2H]C([2H])([2H])C1(C([2H])([2H])[2H])c2ccccc2-c2ccc(Nc3ccc(-c4ccccc4)cc3)cc21] |
| 4m | 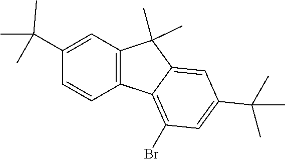 [SMILES: CC(C)(C)c1ccc2c(c1)C(C)(C)c1cc(C(C)(C)C)cc(Br)c1-2] | 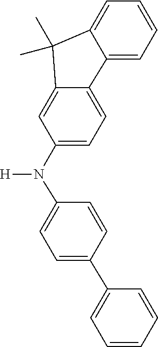 [SMILES: CC1(C)c2ccccc2-c2ccc(Nc3ccc(-c4ccccc4)cc3)cc21] |
| 4n | 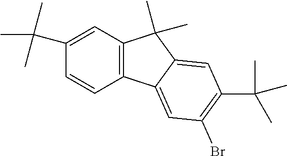 [SMILES: CC(C)(C)c1ccc2c(c1)C(C)(C)c1cc(C(C)(C)C)c(Br)cc1-2] | 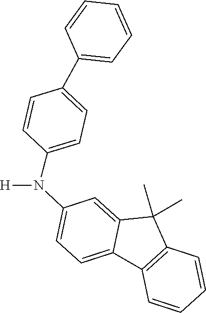 [SMILES: CC1(C)c2ccccc2-c2ccc(Nc3ccc(-c4ccccc4)cc3)cc21] |
| 4o | 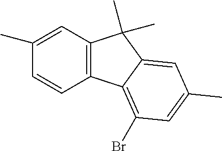 | 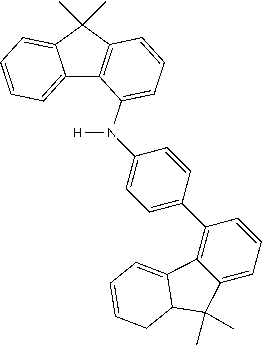 [SMILES: CC1(C)c2ccccc2-c2c(Nc3ccc(-c4cccc5c4C4=CC=CCC4C5(C)C)cc3)cccc21] |
| 4p | 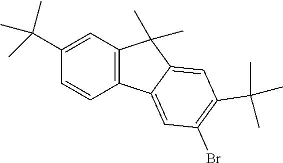 [SMILES: CC(C)(C)c1ccc2c(c1)C(C)(C)c1cc(C(C)(C)C)c(Br)cc1-2] | 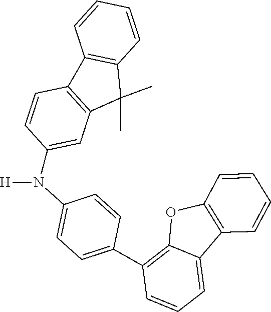 [SMILES: CC1(C)c2ccccc2-c2ccc(Nc3ccc(-c4cccc5c4oc4ccccc45)cc3)cc21] |
| 4q | 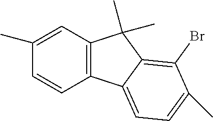 | 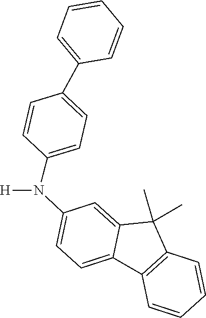 [SMILES: CC1(C)c2ccccc2-c2ccc(Nc3ccc(-c4ccccc4)cc3)cc21] |
| 4r | 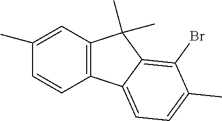 | 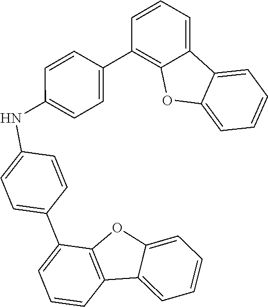 [SMILES: c1ccc2c(c1)oc1c(-c3ccc(Nc4ccc(-c5cccc6c5oc5ccccc56)cc4)cc3)cccc12] |
| 4s | 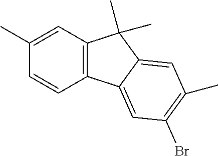 | 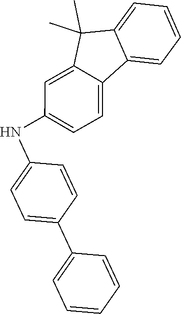 [SMILES: CC1(C)c2ccccc2-c2ccc(Nc3ccc(-c4ccccc4)cc3)cc21] |
| 4t | 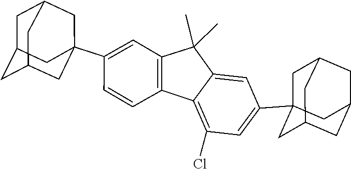 [SMILES: CC1(C)c2cc(C34CC5CC(CC(C5)C3)C4)ccc2-c2c(Cl)cc(C34CC5CC(CC(C5)C3)C4)cc21] | 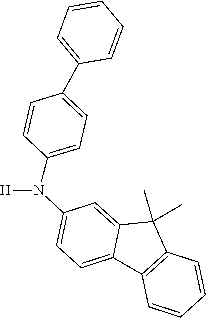 [SMILES: CC1(C)c2ccccc2-c2ccc(Nc3ccc(-c4ccccc4)cc3)cc21] |
| 4u | 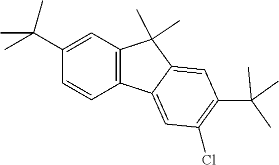 [SMILES: CC(C)(C)c1ccc2c(c1)C(C)(C)c1cc(C(C)(C)C)c(Cl)cc1-2] | 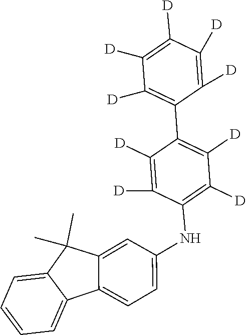 [SMILES: [2H]c1c([2H])c([2H])c(-c2c([2H])c([2H])c(Nc3ccc4c(c3)C(C)(C)c3ccccc3-4)c([2H])c2[2H])c([2H])c1[2H]] |
| 4v | 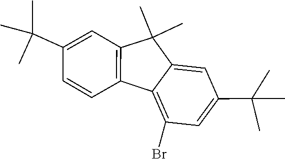 [SMILES: CC(C)(C)c1ccc2c(c1)C(C)(C)c1cc(C(C)(C)C)cc(Br)c1-2] | 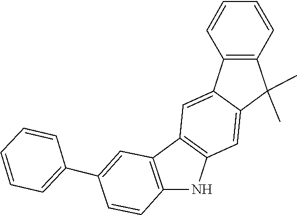 [SMILES: CC1(C)c2ccccc2-c2cc3c(cc21)[nH]c1ccc(-c2ccccc2)cc13] |
| 4w | 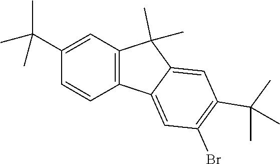 [SMILES: CC(C)(C)c1ccc2c(c1)C(C)(C)c1cc(C(C)(C)C)c(Br)cc1-2] | 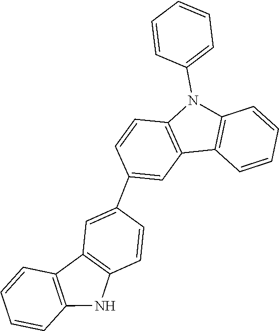 [SMILES: c1ccc(-n2c3ccccc3c3cc(-c4ccc5[nH]c6ccccc6c5c4)ccc32)cc1] |

| Ex. | Product |
|---|---|
| 4b | 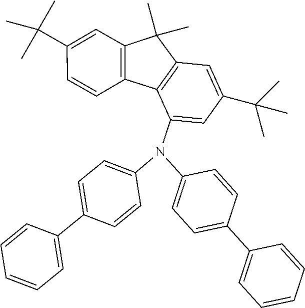 [SMILES: CC(C)(C)c1ccc2c(c1)C(C)(C)c1cc(C(C)(C)C)cc(N(c3ccc(-c4ccccc4)cc3)c3ccc(-c4ccccc4)cc3)c1-2] |
| 4c | 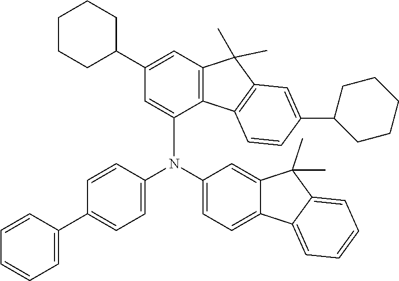 [SMILES: CC1(C)c2ccccc2-c2ccc(N(c3ccc(-c4ccccc4)cc3)c3cc(C4CCCCC4)cc4c3-c3ccc(C5CCCCC5)cc3C4(C)C)cc21] |
| 4d | 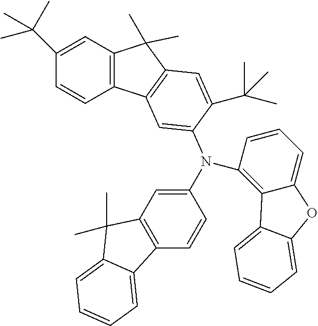 [SMILES: CC(C)(C)c1ccc2c(c1)C(C)(C)c1cc(C(C)(C)C)c(N(c3ccc4c(c3)C(C)(C)c3ccccc3-4)c3cccc4oc5ccccc5c34)cc1-2] |
| 4e | 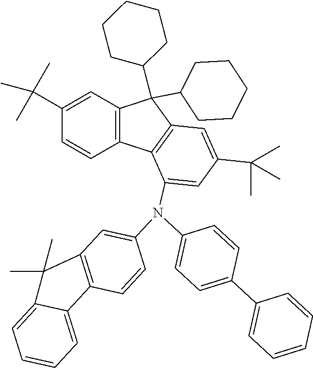 [SMILES: CC(C)(C)c1ccc2c(c1)C(C1CCCCC1)(C1CCCCC1)c1cc(C(C)(C)C)cc(N(c3ccc(-c4ccccc4)cc3)c3ccc4c(c3)C(C)(C)c3ccccc3-4)c1-2] |
| 4f | 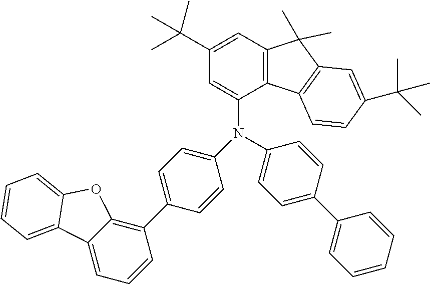 [SMILES: CC(C)(C)c1ccc2c(c1)C(C)(C)c1cc(C(C)(C)C)cc(N(c3ccc(-c4ccccc4)cc3)c3ccc(-c4cccc5c4oc4ccccc45)cc3)c1-2] |
| 4g | 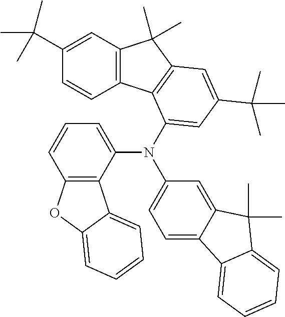 [SMILES: CC(C)(C)c1ccc2c(c1)C(C)(C)c1cc(C(C)(C)C)cc(N(c3ccc4c(c3)C(C)(C)c3ccccc3-4)c3cccc4oc5ccccc5c34)c1-2] |
| 4h | 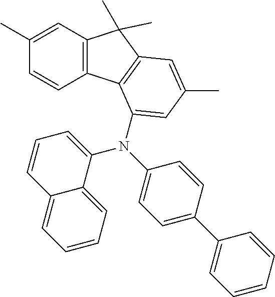 [SMILES: Cc1ccc2c(c1)C(C)(C)c1cc(C)cc(N(c3ccc(-c4ccccc4)cc3)c3cccc4ccccc34)c1-2] |
| 4i | 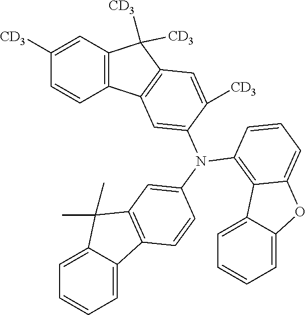 [SMILES: [2H]C([2H])([2H])c1ccc2c(c1)C(C([2H])([2H])[2H])(C([2H])([2H])[2H])c1cc(C([2H])([2H])[2H])c(N(c3ccc4c(c3)C(C)(C)c3ccccc3-4)c3cccc4oc5ccccc5c34)cc1-2] |
| 4j | 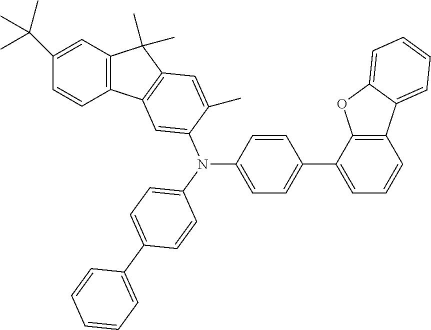 [SMILES: Cc1cc2c(cc1N(c1ccc(-c3ccccc3)cc1)c1ccc(-c3cccc4c3oc3ccccc34)cc1)-c1ccc(C(C)(C)C)cc1C2(C)C] |
| 4k | 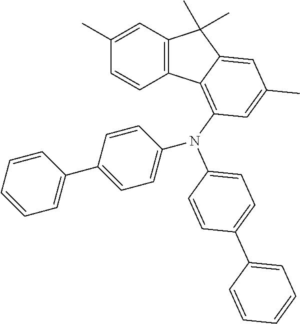 [SMILES: Cc1ccc2c(c1)C(C)(C)c1cc(C)cc(N(c3ccc(-c4ccccc4)cc3)c3ccc(-c4ccccc4)cc3)c1-2] |
| 4l | 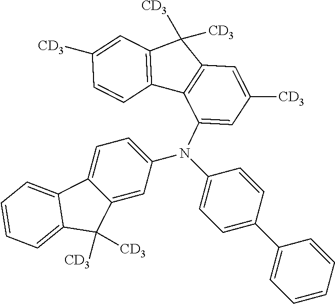 [SMILES: [2H]C([2H])([2H])c1ccc2c(c1)C(C([2H])([2H])[2H])(C([2H])([2H])[2H])c1cc(C([2H])([2H])[2H])cc(N(c3ccc(-c4ccccc4)cc3)c3ccc4c(c3)C(C([2H])([2H])[2H])(C([2H])([2H])[2H])c3ccccc3-4)c1-2] |
| 4m | 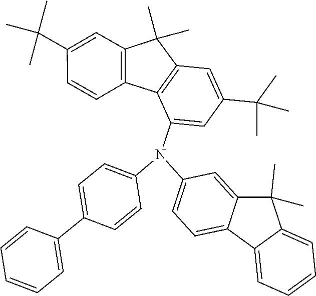 [SMILES: CC(C)(C)c1ccc2c(c1)C(C)(C)c1cc(C(C)(C)C)cc(N(c3ccc(-c4ccccc4)cc3)c3ccc4c(c3)C(C)(C)c3ccccc3-4)c1-2] |
| 4n | 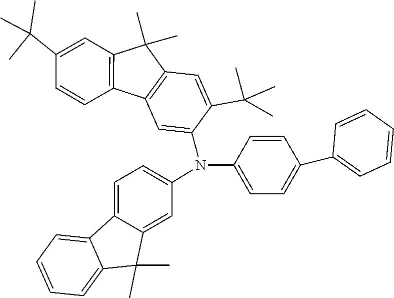 [SMILES: CC(C)(C)c1ccc2c(c1)C(C)(C)c1cc(C(C)(C)C)c(N(c3ccc(-c4ccccc4)cc3)c3ccc4c(c3)C(C)(C)c3ccccc3-4)cc1-2] |
| 4o | 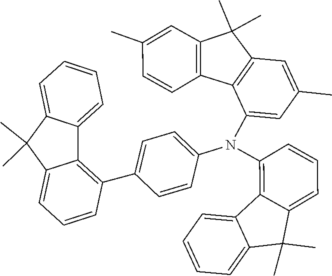 |
| 4p | 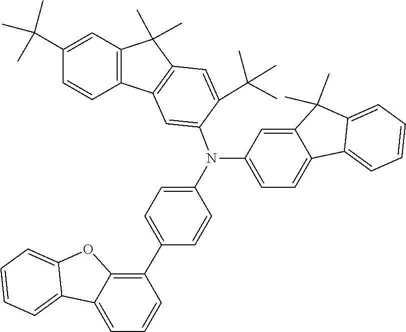 [SMILES: CC(C)(C)c1ccc2c(c1)C(C)(C)c1cc(C(C)(C)C)c(N(c3ccc(-c4cccc5c4oc4ccccc45)cc3)c3ccc4c(c3)C(C)(C)c3ccccc3-4)cc1-2] |
| 4q | 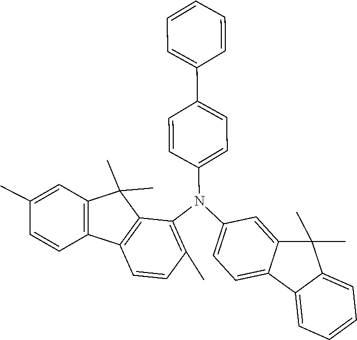 [SMILES: Cc1ccc2c(c1)C(C)(C)c1c-2ccc(C)c1N(c1ccc(-c2ccccc2)cc1)c1ccc2c(c1)C(C)(C)c1ccccc1-2] |
| 4r | 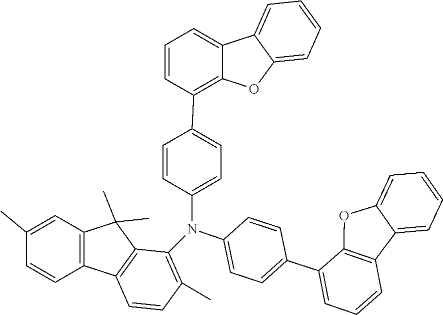 [SMILES: Cc1ccc2c(c1)C(C)(C)c1c-2ccc(C)c1N(c1ccc(-c2cccc3c2oc2ccccc23)cc1)c1ccc(-c2cccc3c2oc2ccccc23)cc1] |
| 4s | 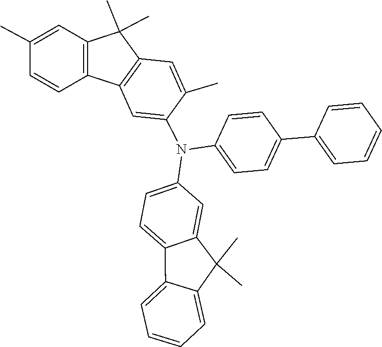 [SMILES: Cc1ccc2c(c1)C(C)(C)c1cc(C)c(N(c3ccc(-c4ccccc4)cc3)c3ccc4c(c3)C(C)(C)c3ccccc3-4)cc1-2] |
| 4t | 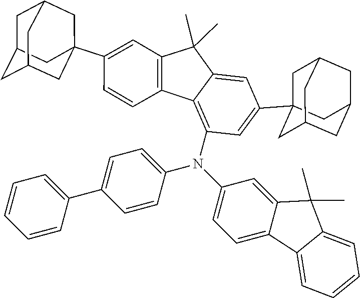 [SMILES: CC1(C)c2ccccc2-c2ccc(N(c3ccc(-c4ccccc4)cc3)c3cc(C45CC6CC(CC(C6)C4)C5)cc4c3-c3ccc(C56CC7CC(CC(C7)C5)C6)cc3C4(C)C)cc21] |
| 4u | 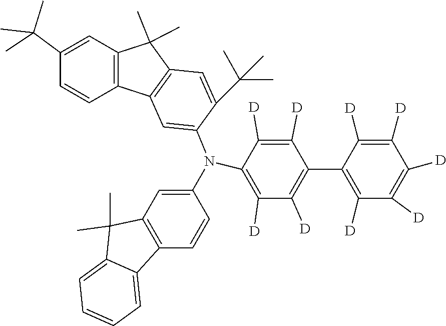 [SMILES: [2H]c1c([2H])c([2H])c(-c2c([2H])c([2H])c(N(c3ccc4c(c3)C(C)(C)c3ccccc3-4)c3cc4c(cc3C(C)(C)C)C(C)(C)c3cc(C(C)(C)C)ccc3-4)c([2H])c2[2H])c([2H])c1[2H]] |
| 4v | 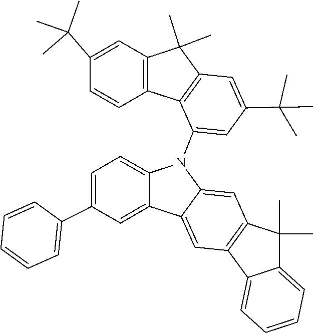 [SMILES: CC(C)(C)c1ccc2c(c1)C(C)(C)c1cc(C(C)(C)C)cc(-n3c4ccc(-c5ccccc5)cc4c4cc5c(cc43)C(C)(C)c3ccccc3-5)c1-2] |
| 4w | 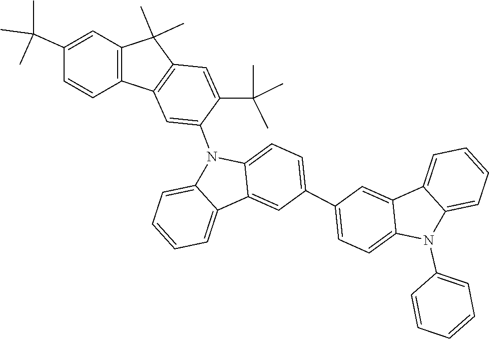 [SMILES: CC(C)(C)c1ccc2c(c1)C(C)(C)c1cc(C(C)(C)C)c(-n3c4ccccc4c4cc(-c5ccc6c(c5)c5ccccc5n6-c5ccccc5)ccc43)cc1-2] |

### Synthesis of N-{[1,1′-biphenyl]-4-yl}-N-[4-(7-tert-butyl-2,9,9-trimethyl-9H-fluoren-4-yl)phenyl]-9,9-dimethyl-9H-fluorene-2-amine 5a

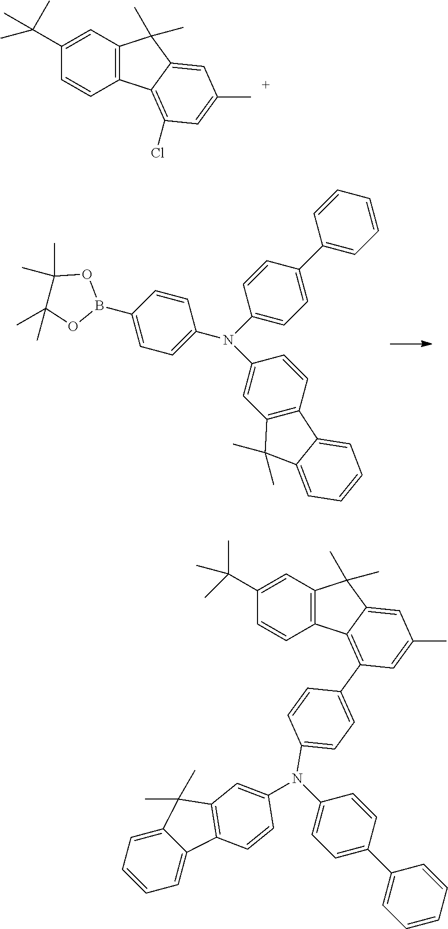 [SMILES: Cc1cc(Cl)c2c(c1)C(C)(C)c1cc(C(C)(C)C)ccc1-2 ; CC1(C)c2ccccc2-c2ccc(N(c3ccc(B4OC(C)(C)C(C)(C)O4)cc3)c3ccc(-c4ccccc4)cc3)cc21 ; Cc1cc(-c2ccc(N(c3ccc(-c4ccccc4)cc3)c3ccc4c(c3)C(C)(C)c3ccccc3-4)cc2)c2c(c1)C(C)(C)c1cc(C(C)(C)C)ccc1-2]

20.0 g (39 mmol) of N-{[1,1′-biphenyl]-4-yl}-9,9-dimethyl-N-[4-(4,4,5,5-tetramethyl-1,3,2-dioxaborolan-2-yl)phenyl]-9H-fluorene-2-amine and 12.8 g (42.9 mmol) of 7-tert-butyl-4-chloro-2,9,9-trimethyl-9H-fluorene are suspended in 400 ml of dioxane and 13.7 g of cesium fluoride (90 mmol). 4.0 g (5.4 mmol) of bis(tricyclohexylphosphine)palladium dichloride is added to this suspension, and the reaction mixture is heated under reflux for 18 h. After cooling, the organic phase is removed, filtered through silica gel, washed three times with 80 ml of water and then concentrated to dryness. After the crude product has been filtered through silica gel with toluene, the remaining residue is recrystallized from heptane/toluene and finally sublimed under high vacuum; purity is 99.9%. The yield is 11 g (40% of theory).

The following compounds are prepared in an analogous manner:

| Ex. | Halide | Amine |
|---|---|---|
| 5b | 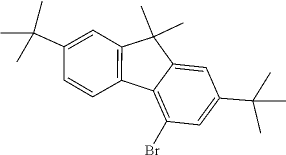 [SMILES: CC(C)(C)c1ccc2c(c1)C(C)(C)c1cc(C(C)(C)C)cc(Br)c1-2] | 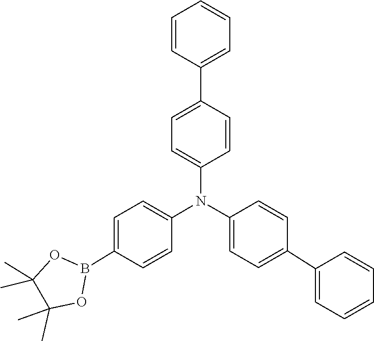 [SMILES: CC1(C)OB(c2ccc(N(c3ccc(-c4ccccc4)cc3)c3ccc(-c4ccccc4)cc3)cc2)OC1(C)C] |
| 5c | 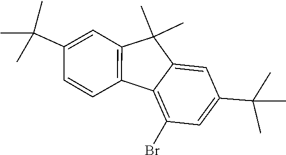 [SMILES: CC(C)(C)c1ccc2c(c1)C(C)(C)c1cc(C(C)(C)C)cc(Br)c1-2] | 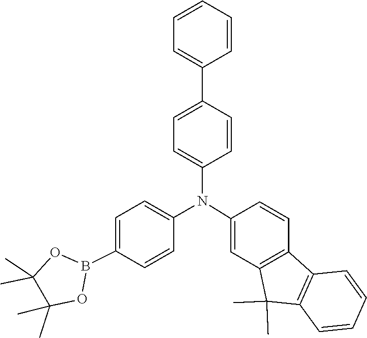 [SMILES: CC1(C)c2ccccc2-c2ccc(N(c3ccc(B4OC(C)(C)C(C)(C)O4)cc3)c3ccc(-c4ccccc4)cc3)cc21] |
| 5d | 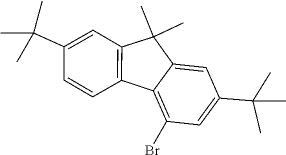 [SMILES: CC(C)(C)c1ccc2c(c1)C(C)(C)c1cc(C(C)(C)C)cc(Br)c1-2] | 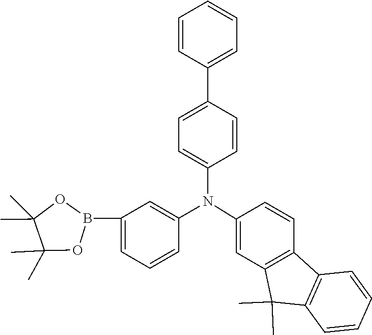 [SMILES: CC1(C)c2ccccc2-c2ccc(N(c3ccc(-c4ccccc4)cc3)c3cccc(B4OC(C)(C)C(C)(C)O4)c3)cc21] |
| 5e | 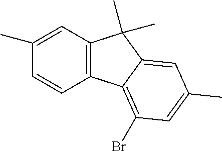 | 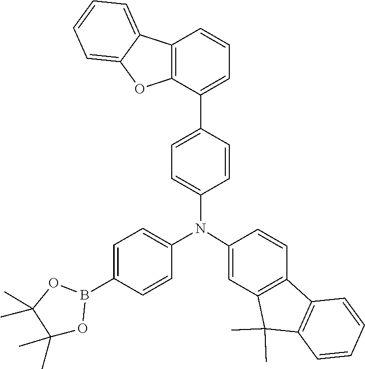 [SMILES: CC1(C)c2ccccc2-c2ccc(N(c3ccc(B4OC(C)(C)C(C)(C)O4)cc3)c3ccc(-c4cccc5c4oc4ccccc45)cc3)cc21] |
| 5f | 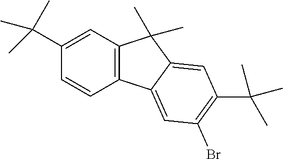 [SMILES: CC(C)(C)c1ccc2c(c1)C(C)(C)c1cc(C(C)(C)C)c(Br)cc1-2] | 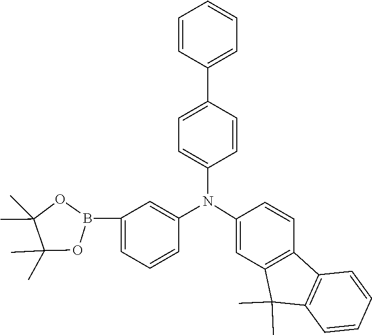 [SMILES: CC1(C)c2ccccc2-c2ccc(N(c3ccc(-c4ccccc4)cc3)c3cccc(B4OC(C)(C)C(C)(C)O4)c3)cc21] |
| 5g | 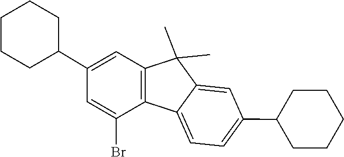 [SMILES: CC1(C)c2cc(C3CCCCC3)ccc2-c2c(Br)cc(C3CCCCC3)cc21] | 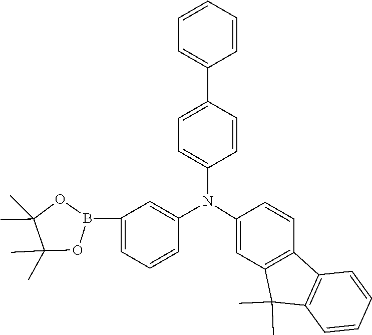 [SMILES: CC1(C)c2ccccc2-c2ccc(N(c3ccc(-c4ccccc4)cc3)c3cccc(B4OC(C)(C)C(C)(C)O4)c3)cc21] |
| 5h | 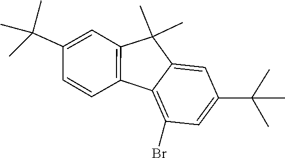 [SMILES: CC(C)(C)c1ccc2c(c1)C(C)(C)c1cc(C(C)(C)C)cc(Br)c1-2] | 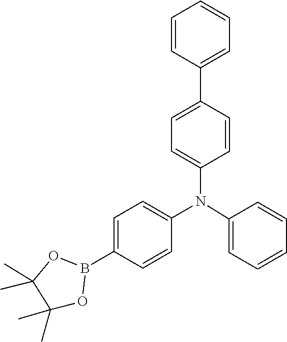 [SMILES: CC1(C)OB(c2ccc(N(c3ccccc3)c3ccc(-c4ccccc4)cc3)cc2)OC1(C)C] |
| 5i | 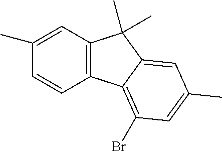 | 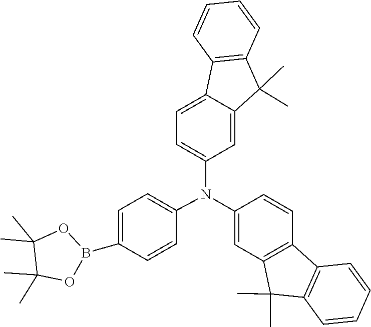 [SMILES: CC1(C)c2ccccc2-c2ccc(N(c3ccc(B4OC(C)(C)C(C)(C)O4)cc3)c3ccc4c(c3)C(C)(C)c3ccccc3-4)cc21] |
| 5j | 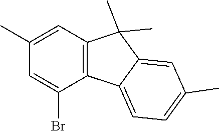 | 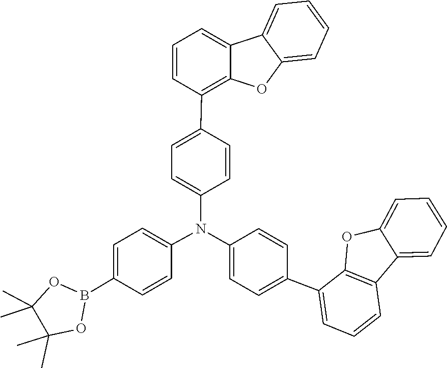 [SMILES: CC1(C)OB(c2ccc(N(c3ccc(-c4cccc5c4oc4ccccc45)cc3)c3ccc(-c4cccc5c4oc4ccccc45)cc3)cc2)OC1(C)C] |
| 5k | 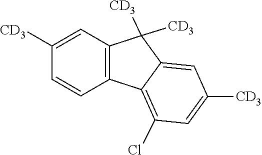 [SMILES: [2H]C([2H])([2H])c1ccc2c(c1)C(C([2H])([2H])[2H])(C([2H])([2H])[2H])c1cc(C([2H])([2H])[2H])cc(Cl)c1-2] | 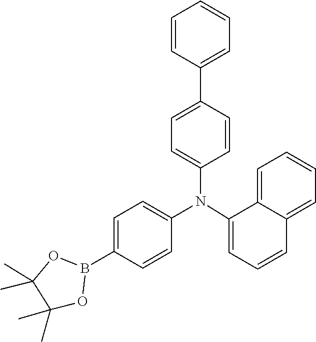 [SMILES: CC1(C)OB(c2ccc(N(c3ccc(-c4ccccc4)cc3)c3cccc4ccccc34)cc2)OC1(C)C] |
| 5l | 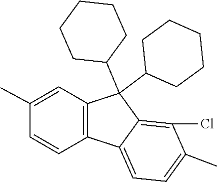 | 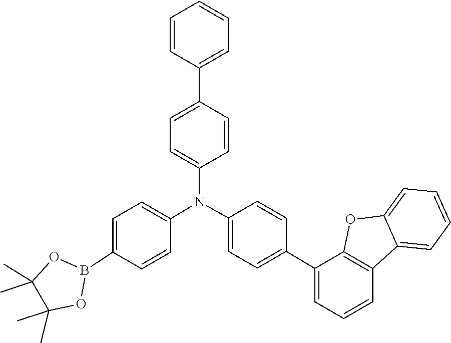 [SMILES: CC1(C)OB(c2ccc(N(c3ccc(-c4ccccc4)cc3)c3ccc(-c4cccc5c4oc4ccccc45)cc3)cc2)OC1(C)C] |
| 5m | 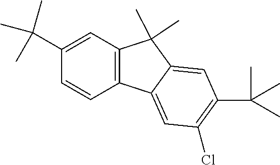 [SMILES: CC(C)(C)c1ccc2c(c1)C(C)(C)c1cc(C(C)(C)C)c(Cl)cc1-2] | 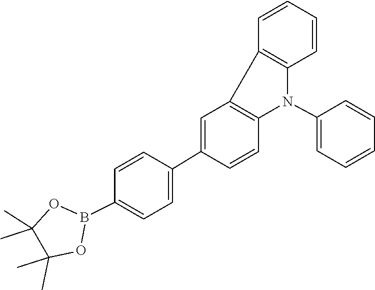 [SMILES: CC1(C)OB(c2ccc(-c3ccc4c(c3)c3ccccc3n4-c3ccccc3)cc2)OC1(C)C] |

| Ex. | Product |
|---|---|
| 5b | 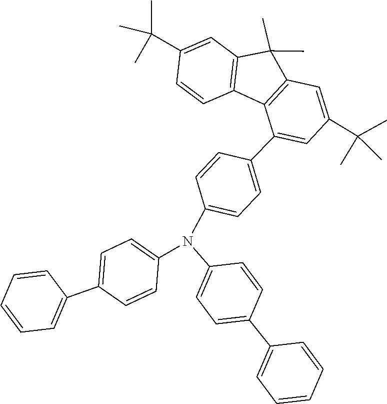 [SMILES: CC(C)(C)c1ccc2c(c1)C(C)(C)c1cc(C(C)(C)C)cc(-c3ccc(N(c4ccc(-c5ccccc5)cc4)c4ccc(-c5ccccc5)cc4)cc3)c1-2] |
| 5c | 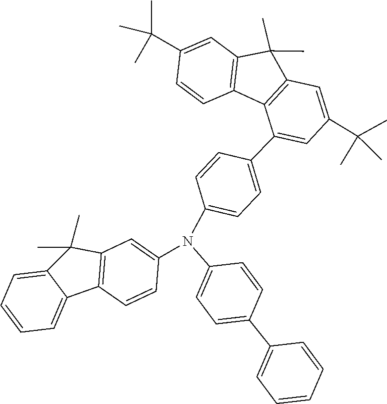 [SMILES: CC(C)(C)c1ccc2c(c1)C(C)(C)c1cc(C(C)(C)C)cc(-c3ccc(N(c4ccc(-c5ccccc5)cc4)c4ccc5c(c4)C(C)(C)c4ccccc4-5)cc3)c1-2] |
| 5d | 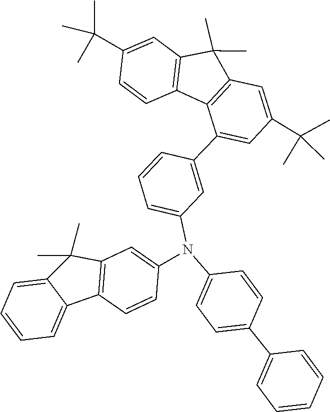 [SMILES: CC(C)(C)c1ccc2c(c1)C(C)(C)c1cc(C(C)(C)C)cc(-c3cccc(N(c4ccc(-c5ccccc5)cc4)c4ccc5c(c4)C(C)(C)c4ccccc4-5)c3)c1-2] |
| 5e | 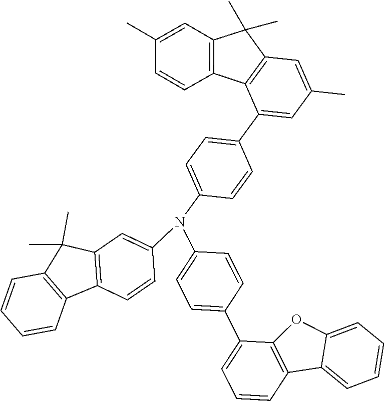 [SMILES: Cc1ccc2c(c1)C(C)(C)c1cc(C)cc(-c3ccc(N(c4ccc(-c5cccc6c5oc5ccccc56)cc4)c4ccc5c(c4)C(C)(C)c4ccccc4-5)cc3)c1-2] |
| 5f | 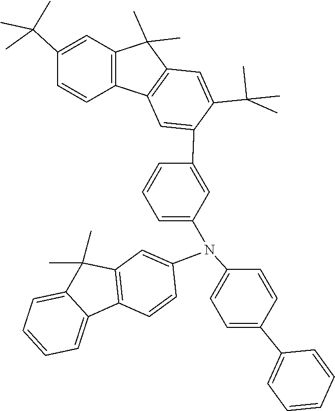 [SMILES: CC(C)(C)c1ccc2c(c1)C(C)(C)c1cc(C(C)(C)C)c(-c3cccc(N(c4ccc(-c5ccccc5)cc4)c4ccc5c(c4)C(C)(C)c4ccccc4-5)c3)cc1-2] |
| 5g | 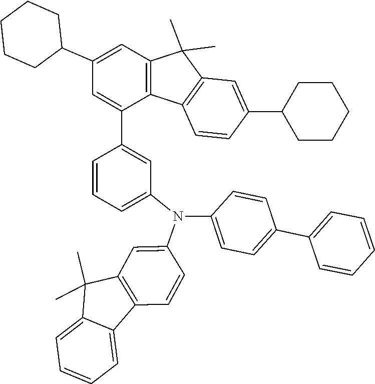 [SMILES: CC1(C)c2ccccc2-c2ccc(N(c3ccc(-c4ccccc4)cc3)c3cccc(-c4cc(C5CCCCC5)cc5c4-c4ccc(C6CCCCC6)cc4C5(C)C)c3)cc21] |
| 5h | 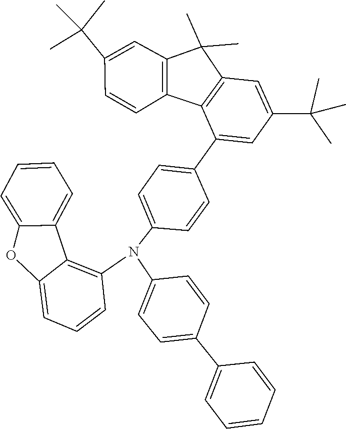 [SMILES: CC(C)(C)c1ccc2c(c1)C(C)(C)c1cc(C(C)(C)C)cc(-c3ccc(N(c4ccc(-c5ccccc5)cc4)c4cccc5oc6ccccc6c45)cc3)c1-2] |
| 5i | 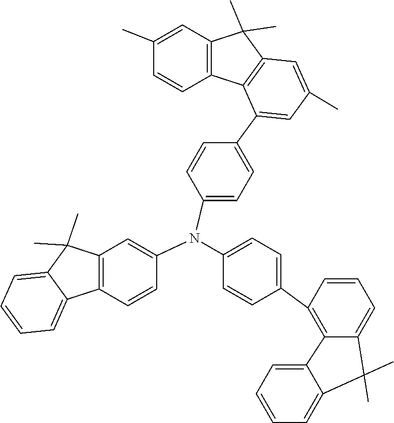 [SMILES: Cc1ccc2c(c1)C(C)(C)c1cc(C)cc(-c3ccc(N(c4ccc(-c5cccc(C)c5-c5ccccc5C)cc4)c4ccc5c(c4)C(C)(C)c4ccccc4-5)cc3)c1-2] |
| 5j | 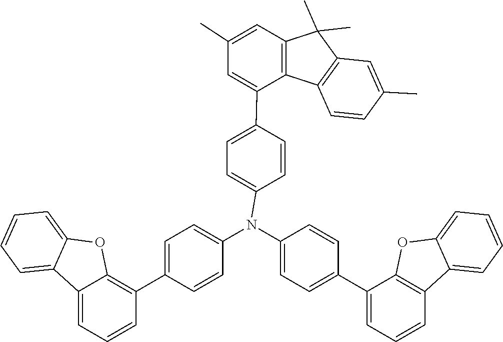 [SMILES: Cc1ccc2c(c1)C(C)(C)c1cc(C)cc(-c3ccc(N(c4ccc(-c5cccc6c5oc5ccccc56)cc4)c4ccc(-c5cccc6c5oc5ccccc56)cc4)cc3)c1-2] |
| 5k | 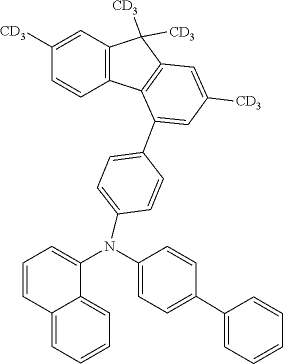 [SMILES: [2H]C([2H])([2H])c1ccc2c(c1)C(C([2H])([2H])[2H])(C([2H])([2H])[2H])c1cc(C([2H])([2H])[2H])cc(-c3ccc(N(c4ccc(-c5ccccc5)cc4)c4cccc5ccccc45)cc3)c1-2] |
| 5l | 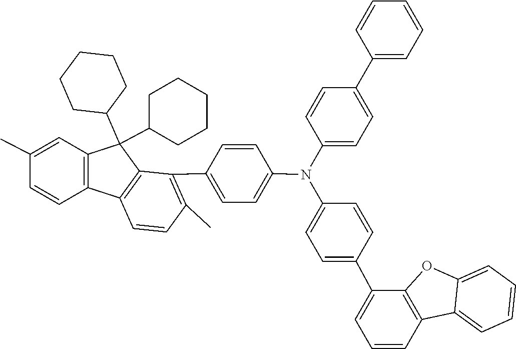 [SMILES: Cc1ccc2c(c1)C(C1CCCCC1)(C1CCCCC1)c1c-2ccc(C)c1-c1ccc(N(c2ccc(-c3ccccc3)cc2)c2ccc(-c3cccc4c3oc3ccccc34)cc2)cc1] |
| 5m | 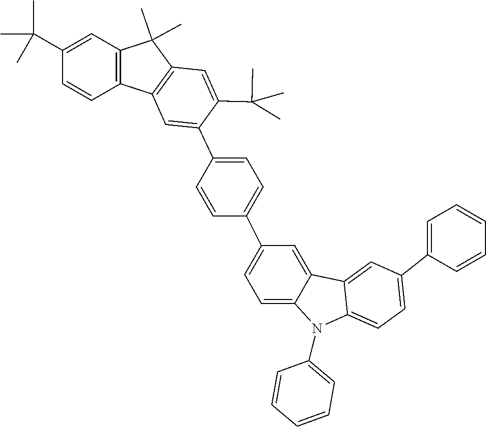 [SMILES: CC(C)(C)c1ccc2c(c1)C(C)(C)c1cc(C(C)(C)C)c(-c3ccc(-c4ccc5c(c4)c4cc(-c6ccccc6)ccc4n5-c4ccccc4)cc3)cc1-2] |

### B) Device Examples

### 1) General Production Process for the OLEDs and Characterization of the OLEDs

Glass plates which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL)/electron blocker layer (EBL)/emission layer (EML)/hole blocker layer (HBL)/electron transport layer (ETL)/electron injection layer (EIL) and finally a cathode. The cathode is formed by an aluminum layer of thickness 100 nm. The exact structure of the OLEDs is shown below. The materials required for production of the OLEDs are shown in a table below. The “HTM” material used in the HIL and the HTL is a fluorene derivative. The p-dopant used is NDP-9 from Novaled AG, Dresden.

All materials are applied by thermal vapor deposition in a vacuum chamber. In this case, the emission layer consists of at least one matrix material (host material) and an emitting dopant (emitter) which is added to the matrix material(s) in a particular proportion by volume by co-evaporation. Details given in such a form as H:SEB (95%:5%) mean here that the material H is present in the layer in a proportion by volume of 95% and SEB in a proportion of 5%. Analogously, the electron transport layer and the hole injection layer also consist of a mixture of two materials.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @10 mA/cm² refers to the external quantum efficiency which is attained at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @80 mA/cm² means here that the lifetime in question is measured at 80 mA/cm².

### 2) Use of the Compounds in OLEDs

In the structure shown, the compounds according to the application can be used in the EBL, as shown hereinafter for compounds 5d and 4m:

| | HIL | HTL | EBL | EML | HBL | ETL | EIL |
|---|---|---|---|---|---|---|---|
| | Thickness / | Thickness / | Thickness / | Thickness / | Thickness / | Thickness / | Thickness / |
| Ex. | nm | nm | nm | nm | nm | nm | nm |
| 1 | HTM: | HTM | 5 d | TMM-1(32%) | HBM | ETM: | LiQ |
| | p-dopant | 50 | 30 nm | TMM-2(60%) | 5 nm | LiQ(50%) | 1 nm |
| | (5%) | nm | | TEG (8%) | | 30 nm | |
| | 10 nm | | | 35 nm | | | |
| 2 | HTM: | HTM | 4 m | TMM-1(32%) | HBM | ETM: | LiQ |
| | p-dopant | 50 nm | 30 nm | TMM-2(60%) | 5 nm | LiQ(50%) | 1 nm |
| | (5%) | | | TEG (8%) | | 30 nm | |
| | 10 nm | | | 35 nm | | | |

Very good performance data are obtained here in all cases; see the following table:

TABLE
OLED data

| Ex. | V | EQE | LT90 |
|---|---|---|---|
| 1 | 3.8 | 25.0 | 104 |
| 2 | 4.0 | 24.4 | 46 |

Structures of the compounds

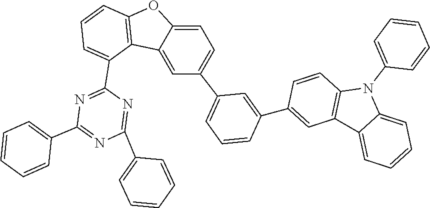 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

TMM-1

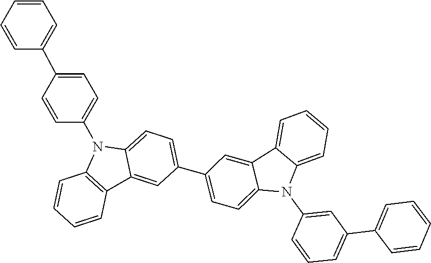 [SMILES: c1ccc(-c2ccc(-n3c4ccccc4c4cc(-c5ccc6c(c5)c5ccccc5n6-c5cccc(-c6ccccc6)c5)ccc43)cc2)cc1]

TMM-2

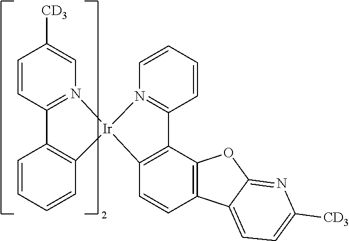

TEG

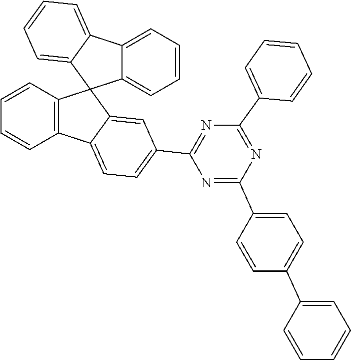 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

ETM

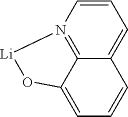

LiQ

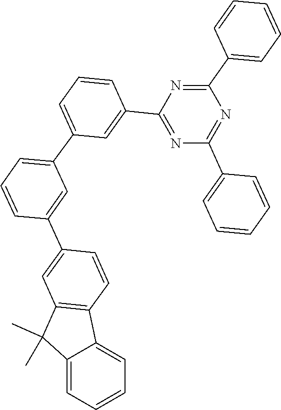 [SMILES: CC1(C)c2ccccc2-c2ccc(-c3cccc(-c4cccc(-c5nc(-c6ccccc6)nc(-c6ccccc6)n5)c4)c3)cc21]

HBM

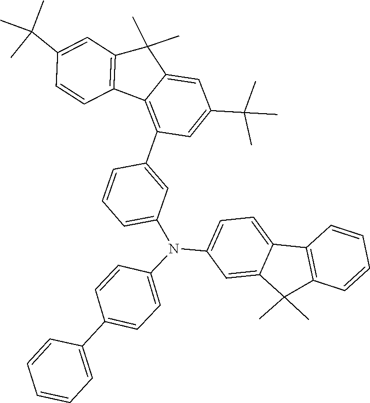 [SMILES: CC(C)(C)c1ccc2c(c1)C(C)(C)c1cc(C(C)(C)C)cc(-c3cccc(N(c4ccc(-c5ccccc5)cc4)c4ccc5c(c4)C(C)(C)c4ccccc4-5)c3)c1-2]

5d

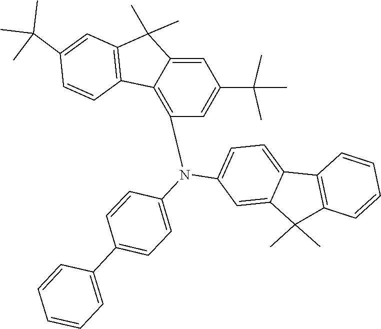 [SMILES: CC(C)(C)c1ccc2c(c1)C(C)(C)c1cc(C(C)(C)C)cc(N(c3ccc(-c4ccccc4)cc3)c3ccc4c(c3)C(C)(C)c3ccccc3-4)c1-2]

4m

1.-20. (canceled)

21. A compound of a formula (I) 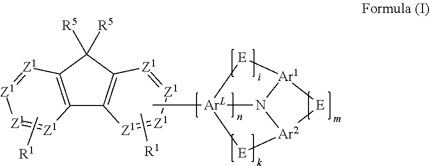
where
Z¹ is C when an R group or a group 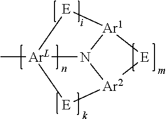
is bonded thereto, and is otherwise the same or different at each instance and is selected from CR² and N;
Ar^{L} is selected from aromatic ring systems which have 6 to 40 aromatic ring atoms and are substituted by R³ radicals and heteroaromatic ring systems which have 5 to 40 aromatic ring atoms and are substituted by R³ radicals;
Ar^{l} is selected from aromatic ring systems which have 6 to 40 aromatic ring atoms and are substituted by R⁴ radicals and heteroaromatic ring systems which have 5 to 40 aromatic ring atoms and are substituted by R⁴ radicals;
Ar² is selected from aromatic ring systems which have 6 to 40 aromatic ring atoms and are substituted by R⁴ radicals and heteroaromatic ring systems which have 5 to 40 aromatic ring atoms and are substituted by R⁴ radicals;
E is a single bond or a divalent group selected from —C(R⁶)₂—, —C(R⁶)₂—C(R⁶)₂—, —C(R⁶)═C(R⁶)—, —N(R⁶)—, —O—, and —S—;
R¹ is the same or different at each instance and is selected from F, CN, N(R⁷)₂, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where said alkyl, alkoxy, alkenyl and alkynyl groups and said aromatic ring systems and heteroaromatic ring systems are each substituted by R⁷ radicals;
R⁵ is the same or different at each instance and is selected from straight-chain alkyl or alkoxy groups having 1 to 20 carbon atoms, branched or cyclic alkyl or alkoxy groups having 3 to 20 carbon atoms, and alkenyl or alkynyl groups having 2 to 20 carbon atoms; where said alkyl, alkoxy, alkenyl and alkynyl groups are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R² is the same or different at each instance and is selected from H, D, F, Cl, Br, I, C(═O)R⁷, CN, Si(R⁷)₃, N(R⁷)₂, NAr¹Ar², P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR₇, —C(═O)O—, —C(═O)NR—, NR⁷, P(═O)(R⁷), —O—, —S—, SO or SO₂;
R³ is the same or different at each instance and is selected from H, D, F, Cl, Br, I, C(═O)R⁷, CN, Si(R⁷)₃, N(R⁷)₂, NAr¹Ar², P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR₇, —C(═O)O—, —C(═O)NR—, NR⁷, P(═O)(R⁷), —O—, —S—, SO or SO₂;
R⁴ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR₇, —C(═O)O—, —C(═O)NR⁷—, NR⁷, P(═O)(R⁷), —O—, —S—, SO or SO₂;
R⁶ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where said alkyl, alkoxy, alkenyl and alkynyl groups and said aromatic ring systems and heteroaromatic ring systems are each substituted by R⁷ radicals; and where one or more CH₂ groups in said alkyl, alkoxy, alkenyl and alkynyl groups may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —5—, SO or SO₂;
R⁷ is the same or different at each instance and is selected from H, D, F, Cl, Br, I, C(═O)R⁸, CN, Si(R⁸)₃, N(R⁸)₂, P(═O)(R⁸)₂, OR⁸, S(═O)R⁸, S(═O)₂R⁸, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁷ radicals may be joined to one another and may form a ring; where said alkyl, alkoxy, alkenyl and alkynyl groups and said aromatic ring systems and heteroaromatic ring systems are each substituted by R⁸ radicals; and where one or more CH₂ groups in said alkyl, alkoxy, alkenyl and alkynyl groups may be replaced by —R⁸C═CR⁸—, —C≡C—, Si(R′)₂, C═O, C═NR′, —C(═O)O—, —C(═O)NR⁸—, NR⁸, P(═O)(R′), —O—, —S—, SO or SO₂;
R⁸ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁸ radicals may be joined to one another and may form a ring; and where said alkyl, alkoxy, alkenyl and alkynyl groups, aromatic ring systems and heteroaromatic ring systems may be substituted by one or more radicals selected from F and CN;
m is 0 or 1, where, when m=0, E is absent, and the Ar¹ and Ar² groups are not bonded to one another;
i is 0 or 1, where, in the case that i=0, the E group in question is absent and the Ar^{L} and Ar¹ groups are not bonded to one another;
k is 0 or 1, where, in the case that k=0, the E group in question is absent and the Ar^{L} and Ar² groups are not bonded to one another;
n is 0 or 1, where, when n=0, Ar^{L} is absent, and i and k are both 0, and the fluorene and the amino group in formula (I) are bonded directly to one another;
where the group 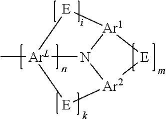
is bonded in the 1, 3 or 4 position of the fluorenyl group of the formula (I),
where the group 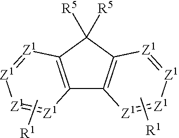
of the formula (I) in the compound is not identical to one or both of the Ar¹ and Ar² groups in the compound; and
where at least one group selected from the Ar¹ and Ar² groups is selected from the following radicals: biphenyl; terphenyl; quaterphenyl; phenanthrenyl; dibenzofuranyl; dibenzothiophenyl;
carbazolyl; and phenyl, which is substituted by at least one group selected from fluorenyl, naphthyl, phenanthrenyl, dibenzofuranyl, dibenzothiophenyl, and carbazolyl, where these radicals are each substituted by R⁴ radicals.

22. The compound as claimed in claim 21, wherein the group 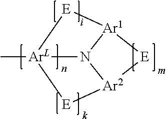
is bonded in the 4 position of the fluorenyl group of the formula (I).

23. The compound as claimed in claim 21, wherein Ar^{L} is phenyl substituted by R³ radicals.

24. The compound as claimed in claim 21, wherein it conforms to one of the following formulae: 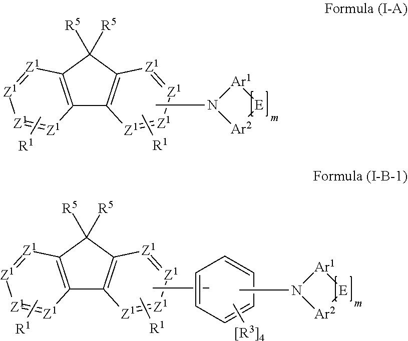
where the groups that occur are as defined in claim 21.

25. The compound as claimed in claim 21, wherein Ar¹ and Ar² are the same or different at each instance and are selected from the radicals benzene, biphenyl, terphenyl, quaterphenyl, naphthyl, fluorenyl, especially 9,9′-dimethylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where said radicals are each substituted by R⁴ radicals.

26. The compound as claimed in claim 21, wherein at least one group selected from the Ar¹ and Ar² groups is a heteroaromatic ring system which has 5 to 40 aromatic ring atoms and is substituted by R⁴ radicals.

27. The compound as claimed in claim 21, wherein Ar¹ and Ar² are selected from biphenyl, terphenyl, quaterphenyl, phenanthrenyl, dibenzofuranyl, dibenzothiophenyl, and carbazolyl, where said groups are each substituted by R⁴ radicals.

28. The compound as claimed in claim 21, wherein Ar¹ and Ar² are selected differently.

29. The compound as claimed in claim 21, wherein it conforms to the following formula: 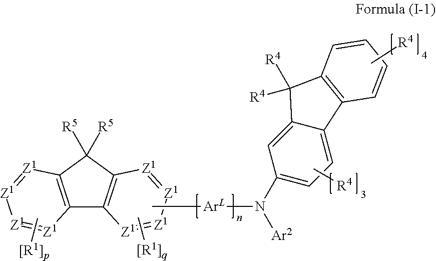
where the groups and indices that occur are as defined in claim 21, and where the R⁴ groups on the benzene rings in the fluorenyl groups are H, and where the —[Ar^{L}]ₙ—N group is bonded in position 1, 3 or 4 of the fluorenyl group and where Ar² is selected from the following radicals: biphenyl; terphenyl; quaterphenyl; phenanthrenyl; dibenzofuranyl; dibenzothiophenyl; carbazolyl; and phenyl, which is substituted by at least one group selected from fluorenyl, naphthyl, phenanthrenyl, dibenzofuranyl, dibenzothiophenyl, and carbazolyl, where these radicals are each substituted by R⁴ radicals.

30. The compound as claimed in claim 21, wherein R¹ is the same or different at each instance and is selected from straight-chain alkyl groups having 1 to 20 carbon atoms, branched or cyclic alkyl groups having 3 to 20 carbon atoms, and aromatic ring systems having 6 to 40 aromatic ring atoms; where said alkyl groups and said aromatic ring systems are each substituted by R⁷ radicals.

31. The compound as claimed in claim 21, wherein it conforms to one of the following formulae: 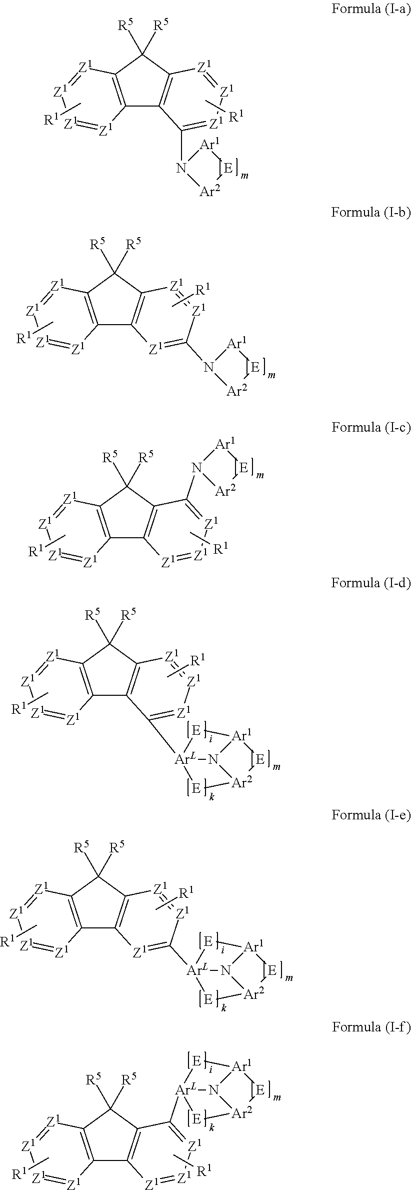
where the groups and indices that occur are as defined in claim 21.

32. The compound as claimed in claim 21, wherein R² is H.

33. The compound as claimed in claim 21, wherein R⁵ is the same or different at each instance and is selected from straight-chain alkyl groups having 1 to 20 carbon atoms, and branched or cyclic alkyl groups having 3 to 20 carbon atoms, where said alkyl groups are each substituted by R⁷ radicals.

34. The compound as claimed in claim 21, wherein it conforms to formula (I) and the variables that occur, in combination together, are as follows:
Z¹ is C when an R¹ group or the group 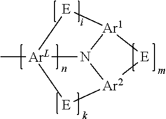
is bonded thereto, and is otherwise CR²;
the group 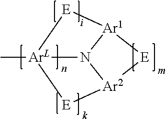
is bonded in the 4 position of the fluorenyl group of the formula (I);
Ar^{L} is phenylene substituted by R³ radicals, where R³ in this case is H;
Ar¹ is selected from aromatic ring systems which have 6 to 40 aromatic ring atoms and are substituted by R⁴ radicals, and heteroaromatic ring systems which have 5 to 40 aromatic ring atoms and are substituted by R⁴ radicals;
Ar² is selected from aromatic ring systems which have 6 to 40 aromatic ring atoms and are substituted by R⁴ radicals, and heteroaromatic ring systems which have 5 to 40 aromatic ring atoms and are substituted by R⁴ radicals;
where at least one group selected from the Ar¹ and Ar² groups is selected from the following radicals: biphenyl; terphenyl; quaterphenyl; phenanthrenyl; dibenzofuranyl; dibenzothiophenyl; carbazolyl; and phenyl substituted by at least one group selected from fluorenyl, naphthyl, phenanthrenyl, dibenzofuranyl, dibenzothiophenyl, and carbazolyl, where these radicals are each substituted by R⁴ radicals;
R¹ is the same or different at each instance and is selected from straight-chain alkyl groups having 1 to 20 carbon atoms, branched or cyclic alkyl groups having 3 to 20 carbon atoms, and aromatic ring systems having 6 to 40 aromatic ring atoms, where said alkyl groups and said aromatic ring systems are each substituted by R⁷ radicals;
R² is H;
R³ is the same or different at each instance and is selected from H, D, F, CN, Si(R⁷)₃, N(R⁷)₂, —NAr¹Ar², straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C═O, C═NR⁷, —NR⁷—, —O—, —S—, —C(═O)O— or —C(═O)NR₇—;
R⁴ and R⁶ are the same or different at each instance and are selected from H, D, F, CN, Si(R⁷)₃, N(R⁷)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C═CR⁷—, Si(R⁷)₂, C═O, C═NR⁷, —NR₇—, —O—, —S—, —C(═O)O— or —C(═O)NR⁷—;
R⁵ is the same or different at each instance and is selected from straight-chain alkyl groups having 1 to 20 carbon atoms, and branched or cyclic alkyl groups having 3 to 20 carbon atoms, where said alkyl groups are each substituted by R⁷ radicals;
R⁷ is the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms;
n is 0 or 1, where, when n=0, Ar^{L} is absent, and the fluorene and the amino group in formula (I) are bonded directly to one another;
i, k and m are 0;
where the group 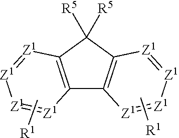
of the formula (I) in the compound is not identical to one or both of the Ar¹ and Ar² groups in the compound.

35. The compound selected from the following compounds:

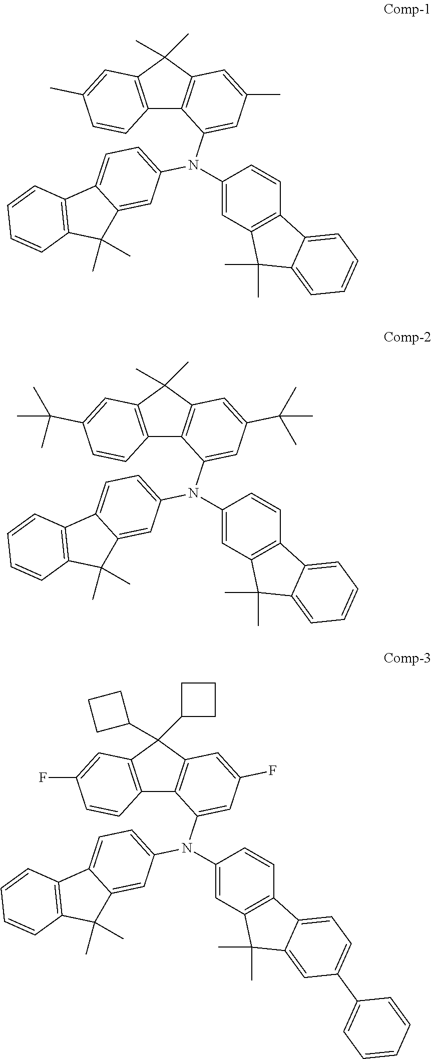 [SMILES: Cc1ccc2c(c1)C(C)(C)c1cc(C)cc(N(c3ccc4c(c3)C(C)(C)c3ccccc3-4)c3ccc4c(c3)C(C)(C)c3ccccc3-4)c1-2 ; CC(C)(C)c1ccc2c(c1)C(C)(C)c1cc(C(C)(C)C)cc(N(c3ccc4c(c3)C(C)(C)c3ccccc3-4)c3ccc4c(c3)C(C)(C)c3ccccc3-4)c1-2 ; CC1(C)c2ccccc2-c2ccc(N(c3ccc4c(c3)C(C)(C)c3cc(-c5ccccc5)ccc3-4)c3cc(F)cc4c3-c3ccc(F)cc3C4(C3CCC3)C3CCC3)cc21]

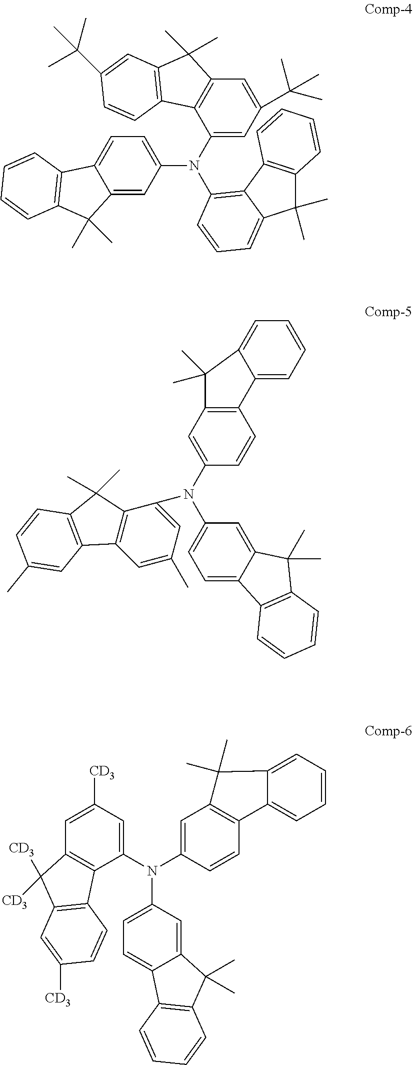

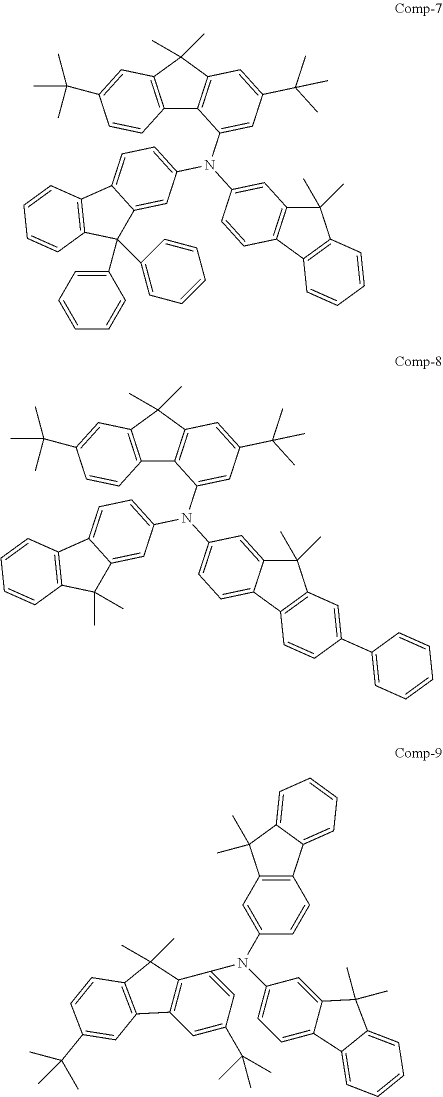 [SMILES: CC(C)(C)c1ccc2c(c1)C(C)(C)c1cc(C(C)(C)C)cc(N(c3ccc4c(c3)C(C)(C)c3ccccc3-4)c3ccc4c(c3)C(c3ccccc3)(c3ccccc3)c3ccccc3-4)c1-2 ; CC(C)(C)c1ccc2c(c1)C(C)(C)c1cc(C(C)(C)C)cc(N(c3ccc4c(c3)C(C)(C)c3ccccc3-4)c3ccc4c(c3)C(C)(C)c3cc(-c5ccccc5)ccc3-4)c1-2 ; CC(C)(C)c1ccc2c(c1)-c1cc(C(C)(C)C)cc(N(c3ccc4c(c3)C(C)(C)c3ccccc3-4)c3ccc4c(c3)C(C)(C)c3ccccc3-4)c1C2(C)C]

36. A process for preparing a compound of the formula (I) as claimed in claim 21, wherein a fluorenyl compound bearing at least one reactive group is either a) reacted with a secondary amine in a Buchwald reaction, or b) reacted with a boronic acid-substituted tertiary amine in a Suzuki reaction, or c) reacted in a sequence of first i) Suzuki reaction with a boronic acid-substituted and halogen-substituted aromatic or heteroaromatic compound, followed by ii) Buchwald reaction of the resultant intermediate with a secondary amine, to give a compound of the formula (I).

37. A formulation comprising at least one compound as claimed in claim 21 and at least one solvent.

38. An electronic device comprising at least one compound as claimed in claim 21.

39. The electronic device as claimed in claim 38, wherein it is an organic electroluminescent device and comprises an anode, cathode and at least one emitting layer, and in that the compound is present in a hole-transporting layer or in an emitting layer of the device.

40. A method comprising including the compound as claimed in claim 21 in an electronic device.
